(19) United States
(12) Patent Application Publication
(10) Pub. No.: US 2007/0281937 A1
(43) Pub. Date: Dec. 6, 2007

(54) Ion Channel Modulators

(76) Inventors: Robert Zelle, Stow, MA (US); Vincent P. Galullo, South Grafton, MA (US); Christopher Todd Baker, Bedford, MA (US); Paul Will, Sudbury, MA (US); William Jack Frazee, Madison, WI (US); Hormoz Mazdiyasni, Marlborough, MA (US); Jinsong Guo, Malden, MA (US)

Correspondence Address: KIRKPATRICK & LOCKHART PRESTON GATES ELLIS LLP;(FORMERLY KIRKPATRICK&LOCKHART NICHOLSON GRAHAM), STATE STREET FINANCIAL CENTER, ONE LINCOLN STREET, BOSTON, MA, 02111-2950, US

(21) Appl. No.: 10/592,451

(22) PCT Filed: Mar. 7, 2005
(86) PCT No.: PCT/US05/07667

§ 371 Date: Jul. 18, 2007

Related U.S. Application Data
(60) Provisional application No. 60/551,510, filed on Mar. 8, 2004.
(60) Provisional application No. 60/551,503, filed on Mar. 8, 2004.
(60) Provisional application No. 60/551,472, filed on Mar. 8, 2004.
(60) Provisional application No. 60/551,473, filed on Mar. 8, 2004.
(60) Provisional application No. 60/551,372, filed on Mar. 8, 2004.
(60) Provisional application No. 60/551,480, filed on Mar. 8, 2004.
(60) Provisional application No. 60/551,395, filed on Mar. 8, 2004.
(60) Provisional application No. 60/551,474, filed on Mar. 8, 2004.
(60) Provisional application No. 60/551,620, filed on Mar. 8, 2004.

(51) Int. Cl. A61K 31/4184; A61K 31/4164; A61K 31/4196; A61K 31/4245; A61K 31/428; A61K 31/4439; A61K 31/454; A61K 31/4709; A61K 31/5377; A61P 9/12; A61P 9/10; A61P 9/00; A61P 25/00; A61P 13/00; A61K 31/497; A61K 31/47; A61K 31/444; A61K 31/433; A61K 31/426; A61K 31/42; A61K 31/4178

(52) U.S. Cl. 514/235.8; 514/254.05; 514/307; 514/314; 514/326; 514/333; 514/338; 514/341; 514/363; 514/364; 514/365; 514/367; 514/378; 514/383; 514/384; 514/395; 514/398

(57) ABSTRACT

The invention relates to compounds, compositions comprising the compounds, and methods of using the compounds and compound compositions. The compounds, compositions, and methods described herein can be used for the therapeutic modulation of ion channel function, and treatment of disease and disease symptoms, particularly those mediated by certain calcium channel subtype targets.

### BACKGROUND

All cells rely on the regulated movement of inorganic ions across cell membranes to perform essential physiological functions. Electrical excitability, synaptic plasticity, and signal transduction are examples of processes in which changes in ion concentration play a critical role. In general, the ion channels that permit these changes are proteinaceious pores consisting of one or multiple subunits, each containing two or more membrane-spanning domains. Most ion channels have selectivity for specific ions, primarily Na⁺, K⁺, Ca²⁺, or Cl⁻, by virtue of physical preferences for size and charge. Electrochemical forces, rather than active transport, drive ions across membranes, thus a single channel may allow the passage of millions of ions per second. Channel opening, or “gating” is tightly controlled by changes in voltage or by ligand binding, depending on the subclass of channel. Ion channels are attractive therapeutic targets due to their involvement in so many physiological processes, yet the generation of drugs with specificity for particular channels in particular tissue types remains a major challenge.

Voltage-gated ion channels open in response to changes in membrane potential. For example, depolarization of excitable cells such as neurons result in a transient influx of Na⁺ ions, which propagates nerve impulses. This change in Na⁺ concentration is sensed by voltage-gated K⁺ channels, which then allow an efflux of K⁺ ions. The efflux of K⁺ ions repolarizes the membrane. Other cell types rely on voltage-gated Ca²⁺ channels to generate action potentials. Voltage-gated ion channels also perform important functions in non-excitable cells, such as the regulation of secretory, homeostatic, and mitogenic processes. Ligand-gated ion channels can be opened by extracellular stimuli such as neurotransmitters (e.g., glutamate, serotonin, acetylcholine), or intracellular stimuli (e.g. cAMP, Ca²⁺, and phosphorylation).

The Caᵥ1 family of voltage-gated calcium channels consists of 4 main subtypes Caᵥ1.1, Caᵥ1.2, Caᵥ1.3 and Caᵥ1.4. These currents are primarily found in skeletal muscle for Caᵥ1.1, heart, smooth muscle, brain, pituitary and adrenal tissue for Caᵥ1.2, brain pancreas, heart, kidney, ovary and cochlea for Caᵥ1.3 and in retina for Caᵥ1.4. These currents require a strong depolarization for activation and are long lasting. The subunit composition of the Caᵥ1 channels is defined by their α₁ subunit, which forms the pore and contains the voltage-sensing gates (α₁1.1, α₁1.2, α₁1.3 and α₁1.4, also known as α_{1S}, α_{1C}, α_{1D}, and α_{1F} respectively) and the β, α₂δ and γ subunits.

Genetic or pharmacological perturbations in ion channel function can have dramatic clinical consequences. Long QT syndrome, epilepsy, cystic fibrosis, and episodic ataxia are a few examples of heritable diseases resulting from mutations in ion channel subunits. Toxic side affects such as arrhythmia and seizure which are triggered by certain drugs are due to interference with ion channel function (Sirois, J. E. and, Atchison, W. D., Neurotoxicology 1996; 17(1):63-84; Keating, M. T., Science 1996 272:681-685). Drugs are useful for the therapeutic modulation of ion channel activity, and have applications in treatment of many pathological conditions, including hypertension, angina pectoris, myocardial ischemia, asthma, bladder overactivity, alopecia, pain, heart failure, dysmenorrhea, type II diabetes, arrhythmia, graft rejection, seizure, convulsions, epilepsy, stroke, gastric hypermotility, psychoses, cancer, muscular dystrophy, and narcolepsy (Coghlan, M. J., et al. J. Med. Chem. 2001, 44:1627-1653; Ackerman. M. J., and Clapham, D. E. N. Eng. J. Med. 1997, 336:1575-1586). The growing number of identified ion channels and understanding of their complexity will assist in future efforts at therapies, which modify ion channel function.

Overactive bladder (OAB) is characterized by storage symptoms such as urgency, frequency and nocturia, with or without urge incontinence, resulting from the overactivity of the detrusor muscle in the bladder. OAB can lead to urge incontinence. The etiology of OAB and painful bladder syndrome is unknown, although disturbances in nerves, smooth muscle and urothelium can cause OAB (Steers, W. Rev Urol, 4:S7-S18). There is evidence to suggest that reduction of bladder hyperactivity may be indirectly effected by inhibition of Caᵥ2.2 and/or Caᵥ1 channels.

### SUMMARY

The invention relates to heterocyclic compounds, compositions comprising the compounds, and methods of using the compounds and compound compositions. The compounds and compositions comprising them are useful for treating disease or disease symptoms, including those mediated by or associated with ion channels.

In one aspect is a compound of formula (AI) or pharmaceutical salt thereof

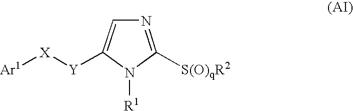

wherein,
Ar¹ is cycloalkyl, aryl, heterocyclyl, or heteroaryl, each optionally substituted with one or more substituents;
X is NR³, C(R³)₂, or O;
Y is C═O or lower alkyl;
R¹ is Ar² or lower alkyl optionally substituted with Ar²;
each Ar² is independently cycloalkyl, aryl, heterocyclyl, or heteroaryl, each optionally substituted with one or more substituents;
q is 0, 1 or 2;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR⁴;
each R³ is independently selected from H, or lower alkyl;
each R⁴ is independently selected from H, lower alkyl or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy.
In other aspects, the compounds are those of any of the formulae herein (including any combinations thereof):
Wherein,
Ar¹ is aryl or heteroaryl, each optionally substituted with one or more substituents;
X is NR³; and
Y is C═O;
Wherein, R¹ is aryl or heteroaryl, each optionally substituted with one or more substituents;
Wherein, each R² is independently (CH₂)ₘAr³; and each Ar³ is heteroaryl optionally substituted with one or more substituents;
Wherein, Ar³ is a heteroaryl comprising a five-membered ring having carbon atoms and 1, 2 or 3 heteroatoms selected from N, O and S, optionally substituted with one or more substituents;

Wherein, Ar³ is pyrrolidinyl, pyrazolyl, imidazolyl, oxazolyl, thiazolyl, benzimidazolyl, benzoxazolyl, or benzthiazolyl, each optionally substituted with one or more substituents;
Wherein each R² is (CH₂)ᵣNR³R⁴, wherein each R⁴ is independently (CH₂)ₚAr³;
Wherein R³ is H;

Wherein the compound of formula AI is a compound delineated in any of the tables herein, or pharmaceutical salt thereof.

In one aspect is a compound of formula (BI) or pharmaceutical salt thereof.

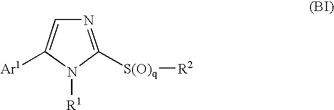

wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
q is 0, 1 or 2;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR⁴;
each R³ is independently selected from H, or lower alkyl;
each R⁴ is independently selected from H, lower alkyl or (CH₂)ₚAr³;
each Ar³ is cycloalkyl, aryl, heterocyclyl, or heteroaryl, each optionally substituted with one or more substituents, with the proviso that Ar³ is not piperidinyl, tetrahydroquinolinyl or tetrahydroisoquinolinyl;
each Z is independently selected from O or NR³;
each m is 1 or 2;
each n is 2 or 3;
each p is 0 or 1;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy.
   In other aspects, the compounds are those of any of the formulae herein (including any combinations thereof):

Wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
q is 0;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR¹;
each R³ is independently selected from H, or lower alkyl;
each R⁴ is independently selected from H, lower alkyl or (CH₂)ₚAr³;
each Ar³ is cycloalkyl, aryl, heterocyclyl, or heteroaryl, each optionally substituted with one or more substituents, with the proviso that Ar³ is not piperidinyl, tetrahydroquinolinyl or tetrahydroisoquinolinyl;
each Z is independently selected from O or NR³;
each m is 1 or 2;
each n is 2 or 3;
each p is 0 or 1;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶., NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy;

Wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
q is 1;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR⁴;
each R³ is independently selected from H, or lower alkyl;
each R⁴ is independently selected from H, lower alkyl or (CH₂)ₚAr³;
each Ar³ is cycloalkyl, aryl, heterocyclyl, or heteroaryl, each optionally substituted with one or more substituents, with the proviso that when R² is (CH₂)ₘAr³ and m is 1, then Ar³ is not ortho dimethylaminophenyl;
each Z is independently selected from O or NR³;
each m is 1 or 2;
each n is 2 or 3;
each p is 0 or 1;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;-
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy;

Wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
q is 2;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR⁴;
each R³ is independently selected from H, or lower alkyl;
each R⁴ is independently selected from H, lower alkyl or (CH₂)ₚAr³;
each Ar³ is cycloalkyl, aryl, heterocyclyl, or heteroaryl, each optionally substituted with one or more substituents;
each Z is independently selected from O or NR³;
each m is 1 or 2;
each n is 2 or 3;
each p is 0 or 1;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy;
   Wherein,
   R² is (CH₂)ₘCO₂R³, (CH₂)ₘCOAr³, or (CH₂)ₘCONR³R⁴, and m is 2;
   Wherein,
   R¹ is Ar² or lower alkyl substituted with Ar², R² is (CH₂)ₘAr³, and m is 1, with the proviso that R¹ is not furylmethyl or tetrahydrofurylmethyl;
   Wherein,
   R¹ is Ar² or lower alkyl substituted with Ar²,
   R² is (CH₂)ₘAr³, and m is 2, with the proviso that R¹ is not furylmethyl or tetrahydrofurylmethyl;
   Wherein,
   Ar¹ and R¹ are each an optionally substituted aryl, and R² is independently selected from (CH₂)ₘAr³, (CH₂)₃Ar³, (CH₂)ₙNR³R⁴ or (CH₂)ₙOR⁴;
   Wherein,
   each R² is independently selected from (CH₂)ₘAr³, and each Ar³ is heteroaryl optionally substituted with one or more substituents;
   Wherein,
   Ar³ is heteroaryl having a five-membered ring of carbon atoms and 1, 2 or 3 heteroatoms selected from N, O and S, optionally substituted with one or more substituents;
   Wherein,
   Ar³ is pyrrolidinyl, pyrazolyl, imidazolyl, thioimidazolyl, benzimidazolyl, or benzthioimidazolyl, each optionally substituted with one or more substituents;

Wherein, the compound of formula BI is a compound delineated in any of the tables herein or pharmaceutical salt thereof.

One aspect is a method of treating a disease or disease symptom in a subject including administering to the subject an effective amount a compound of formula CI or pharmaceutical salt thereof:

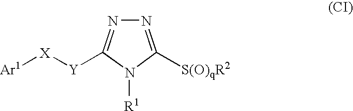

wherein,

Ar¹ is cycloalkyl, aryl, heterocyclyl, or heteroaryl, each optionally substituted with one or more substituents;
X is NR³, C(R³)₂, S, a bond or O, or together with Y forms —CH═CH—;
Y is C═O, a bond, or lower alkyl, or together with X forms —CH═CH—;
R¹ is Ar², alkenyl, or lower alkyl optionally substituted with Ar²;

each Ar² is independently cycloalkyl, aryl, heterocyclyl, or heteroaryl, each optionally substituted with one or more substituents;

q is o, 1 or 2;

each R² is independently (CH₂)ₘCO₂R³, (CH₂)ₘCOAr³, (CH₂)ₘCONR³R⁴, (CH₂)ₘAr³, (CH₂)₃Ar³, (CH₂).NR³R⁴, (CH₂)OR⁴; (CH₂)ₘCN; alkyl; alkynyl, (CR³R³)ₘCONR³R⁴, Ar⁴, (CR³R³)ₘN(R³)C(O)Ar³, or (CH₂)ₘC(NOH)NH₂;

each R³ is independently H, or lower alkyl;

each R⁴ is independently H, lower alkyl, alkoxy, (CH₂), NR⁵R⁶, or (CH₂)ₚAr³;

m is 1 or 2;

n is 2 or 3;

p is 0 or 1;

each Ar³ is cycloalkyl, aryl, heterocyclyl, or heteroaryl, each optionally substituted with one or more substituents;

each substituent for Ar¹, Ar² and Ar³ is independently halogen, CN, NO₂, OR⁶, SR⁶, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;

each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl;

each R⁶ is independently hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl;

each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl; and

each Ar⁴ is independently C₃-C₆ cycloalkyl, heterocyclyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy.

In other aspects, the methods are those of any of the formulae herein (including any combinations thereof):

Wherein,

Ar¹ is aryl or heteroaryl, each optionally substituted with one or more substituents;

X is NR³;

Y is lower alkyl;

R¹ is aryl optionally substituted with one or more substituents; and

each R² is independently (CH₂)ₘCO₂R³, (CH₂)ₘCOAr³, (CH₂)ₘCONR³R⁴, (CH₂)ₘAr³, (CH₂)₃Ar³, or (CH₂)ₙNR³R⁴.

Wherein,

Ar¹ is aryl or heteroaryl, each optionally substituted with one or more substituents;

X is a bond;

Y is a bond;

R¹ is aryl optionally substituted with one or more substituents; and

each R² is independently selected from (CH₂,)ₘCO₂R³, (CH₂)ₘCOAr³, (CH₂)ₘCONR³R⁴, (CH₂)ₘAr³, (CH₂)₃Ar³, (CH₂)NR³R⁴;

Wherein, each R² is independently selected from (CH₂)ₘAr³;

Wherein,

each R² is independently selected from (CH₂)ₘAr³; and

each Ar³ is heteroaryl optionally substituted with one or more substituents;

Wherein Ar³ is a heteroaryl comprising a five-membered ring having carbon atoms and 1, 2 or 3 heteroatoms selected from N, O and S, optionally substituted with one or more substituents;

Wherein Ar³ is pyrrolidinyl, pyrazolyl, imidazolyl, oxazolyl, thiazolyl, benzimidazolyl, benzoxazolyl, or benzthiazolyl, each optionally substituted with one or more substituents;

Wherein the compound of formula CI is a compound delineated in any of Tables C1, or pharmaceutical salt thereof;

Another aspect is a method of modulating calcium channel activity comprising contacting a calcium channel with a compound of any of the formulae herein.

Another aspect is a compound of formula CI above, or pharmaceutical salt thereof.

Another aspect is a compound of formula CI or pharmaceutical salt thereof, 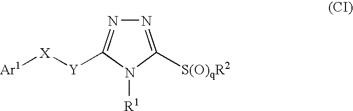
wherein,

Ar¹ is cycloalkyl, aryl, heterocyclyl, or heteroaryl, each optionally substituted with one or more substituents, and each attached to X by a carbon atom;

X is CH₂;

Y is a bond;

R¹ is Ar², alkenyl, or lower alkyl optionally substituted with Ar²;

each Ar² is independently cycloalkyl, aryl, heterocyclyl, or heteroaryl, each optionally substituted with one or more substituents;

q is o, 1 or 2;

each R² is independently (CH₂)ₘCO₂R³, (CH₂)ₘCOAr³, or (CH₂)ₘCONR³R⁴;

each R³ is independently H, or lower alkyl;

each R⁴ is independently H, lower alkyl, alkoxy, (CH₂). NR⁵R⁶, or (CH₂)ₚAr³;

m is 2;

n is 2 or 3;

p is 0 or 1;

each Ar³ is cycloalkyl, aryl, heterocyclyl, or heteroaryl, each optionally substituted with one or more substituents;

each substituent for Ar¹, Ar² and Ar³ is independently halogen, CN, NO₂, OR⁶, SR⁶, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, c(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;

each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl;

each R⁶ is independently hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl;

each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino or C₃-C₆ cycloalkyl; and

each Ar⁴ is independently C₃-C₆ cycloalkyl, heterocyclyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy.

In other aspects, the compounds are those of any of the formulae herein (including any combinations thereof):

Wherein,

Ar¹ is cycloalkyl, aryl, heterocyclyl, or heteroaryl, each optionally substituted with one or more substituents, and each attached to X by a carbon atom;

X is a bond;

Y is a bond;

R¹ is Ar²;

each Ar² is independently cycloalkyl, aryl, heterocyclyl, or heteroaryl, each optionally substituted with one or more substituents;

each R² is 4-pyridylmethyl;

Wherein,

Ar¹ is cycloalkyl, aryl, heterocyclyl, or heteroaryl, each optionally substituted with one or more substituents, and each attached to X by a carbon atom, however, Ar¹ is not 4-pyridyl;

X is a bond;

Y is a bond;

R¹ is Ar²;

each Ar² is independently cycloalkyl, aryl, heterocyclyl, or heteroaryl, each optionally substituted with one or more substituents; and

each R² is 3-pyridylmethyl;

Wherein,

Ar¹ is cycloalkyl, aryl, heterocyclyl, or heteroaryl, each optionally substituted with one or more substituents, and each attached to X by a carbon atom;

X is a bond;

Y is a bond;

R¹ is Ar²;

each Ar² is independently cycloalkyl, aryl, heterocyclyl, or heteroaryl, each optionally substituted with one or more substituents; and

each R² is 2-pyridylmethyl;

Wherein:

Ar¹ is cycloalkyl, aryl, heterocyclyl, or heteroaryl, each optionally substituted with one or more substituents;

X is a bond;

Y is a bond;

R¹ is Ar²;

each Ar² is independently cycloalkyl, aryl, heterocyclyl, or heteroaryl, each optionally substituted with one or more substituents; and

each R² is:

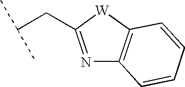

wherein W is NR³,S or O.

Wherein,

Ar¹ is cycloalkyl, aryl, heterocyclyl, or heteroaryl, each optionally substituted with one or more substituents;

X is a bond;

Y is a bond;

R¹ is Ar²;

each Ar² is independently cycloalkyl, aryl, heterocyclyl, or heteroaryl, each optionally substituted with one or more substituents;

each R² is (CH₂)ₘAr³; and

each Ar³ is aryl substituted with NH₂, S(O)₂OR³, COOH, or C(O)NH₂;

One aspect is a compound of formula D-(I) or pharmaceutical salt thereof

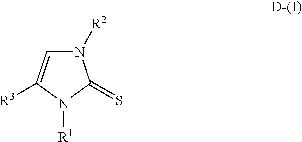

wherein,
R³ is Ar¹ or Ar¹—X—Y wherein,
   each Ar¹ is cycloalkyl, aryl, heterocyclyl, or heteroaryl, each optionally substituted with one or more substituents;
   X is NR⁴, C(R⁴)₂, or O;
   Y is C═O or lower alkyl;
R¹ is Ar² or lower alkyl optionally substituted with Ar²;
each Ar² is independently cycloalkyl, aryl, heterocyclyl, or heteroaryl, each optionally substituted with one or more substituents;
each R² is independently selected from (CH₂)ₘC(O)OR⁴, (CH₂)ₘC(O)Ar³, (CH₂)ₘC(O)NR⁴R⁵, (CH₂)ₙNR⁴R⁵, (CH₂)₃Ar³, or (CH₂)ₘAr³;
each R⁴ is independently selected from H, or lower alkyl;
each R⁵ is independently selected from H, lower alkyl or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁶, SR⁶, S(O)₂OR⁶, NR⁶R⁷, cycloalkyl, C₁-C₂ perfluoroalkyl, C₁-C₂ perfluoroalkoxy, 1,2-methylenedioxy, C(O)OR⁶, C(O)NR⁶R⁷, OC(O)NR⁶R⁷., NR⁶C(O)NR⁶R⁷, C(NR⁶)NR⁶R⁷, NR⁶C(NR⁷)NR⁶R⁷, S(O)₂NR⁶R⁷, R⁸, C(O)R⁸, NR⁶C(O)R⁸, S(O)R⁸, or S(O)₂R⁸;
each R⁶ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently selected from (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl; and
q is 0 or 1.
Another aspect is a compound of any of the formulae herein (including any combinations thereof),
wherein R³ is Ar¹ and R¹ is Ar²;
wherein
   R³ is independently, aryl or heteroaryl, each optionally substituted with one or more substituents; and
   R¹ is independently, aryl or heteroaryl, each optionally substituted with one or more substituents;
wherein R² is (CH₂)ₘC(O)OR⁴, (CH₂)ₘC(O)Ar³ or (CH₂)ₘC(O)NR⁴R⁵;
wherein R² is (CH₂)ₘAr³ and Ar³ is aryl or heteroaryl each optionally substituted with one or more substituents;
wherein R² is (CH₂)ₘC(O)NR⁴R⁵ and R⁵ is independently (CH₂)ₚAr³, wherein
Ar³ is aryl or heteroaryl, each optionally substituted with one or more substituents;
wherein R² is (CH₂)ₙNR⁴R⁵ or (CH₂)ₘAr³;

wherein m is 2 and Ar³ is a heteroaryl comprising a five-membered ring having carbon atoms and 1, 2 or 3 heteroatoms selected from N, O and S, optionally substituted with one or more substituents;

wherein Ar³ is pyrrolidinyl, pyrazolyl, imidazolyl, oxazolyl, thiazolyl, benzimidazolyl, benzoxazolyl, or benzthiazolyl, each optionally substituted with one or more substituents; or
wherein the compound of formula D-I is a compound of any of Tables D-(1-6).
One aspect is a compound of formula E-(I) or pharmaceutical salt thereof

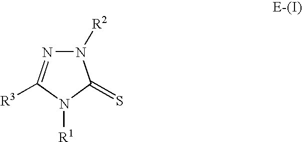

wherein,
R³ is alkyl, alkoxyalkyl, Ar¹ or Ar¹—X—Y wherein,
   each Ar¹ is independently cycloalkyl, aryl, heterocyclyl, or heteroaryl, each optionally substituted with one or more substituents;
   X is NR⁴, C(R⁴)₂, or O;
   Y is C═O or lower alkyl;
R¹ is H, alkenyl, Ar² or lower alkyl optionally substituted with Ar²
each Ar² is independently cycloalkyl, aryl, heterocyclyl, or heteroaryl, each optionally substituted with one or more substituents;
each R² is independently selected from H, (CH₂)ₘC(O)OR⁴, (CH₂)ₘC(O)Ar³, (CH₂)ₘC(O)NR⁴R⁵, (CH₂)ₘC(O)N(OR⁴)R⁵, (CH₂)ₘCH₂OR⁴, Ar³, (CH₂)ₙAr³; (CH₂)ₙNR⁴R⁵, or (CH₂)ₘAr³;
each R⁴ is independently selected from H, or lower alkyl;
each R⁵ is independently selected from H, lower alkyl or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁶, SR⁶, S(O)₂OR⁶, NR⁶R⁷, cycloalkyl, C₁-C₂ perfluoroalkyl, C₁-C₂ perfluoroalkoxy, 1,2-methylenedioxy, C(O)OR⁶, C(O)NR⁶R⁷, OC(O)NR⁶R⁷. NR⁶C(O)NR⁶R⁷, C(NR⁶)NR⁶R⁷, NR⁶C(NR⁷)NR⁶R⁷, S(O)₂NR⁶R⁷, R⁸, C(O)R⁸, NR⁶C(O)R⁸, S(O)R⁸, or S(O)₂R⁸;
each R⁶ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently selected from (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl; and
q is 0 or 1.
Another aspect is a compound of any of the formulae herein (including any combinations thereof):
wherein, R³ is Ar¹ and R¹ is Ar²;
wherein,
   R³ is independently, aryl or heteroaryl, each optionally substituted with one or more substituents; and
   R¹ is independently, aryl or heteroaryl, each optionally substituted with one or more substituents;
wherein R² is (CH₂)ₘC(O)OR⁴, (CH₂)ₘC(O)Ar³ or (CH₂)ₘC(O)NR⁴R⁵;
wherein R² is (CH₂)ₘAr³ and Ar³ is aryl or heteroaryl each optionally substituted with one or more substituents;
wherein R² is (CH₂)ₘC(O)NR⁴R⁵ and R⁵ is independently (CH₂)ₚAr³, wherein Ar³ is aryl or heteroaryl, each optionally substituted with one or more substituents;
wherein R² is (CH₂)ᵣNR⁴R⁵ or (CH₂)ₘAr³; or
wherein the compound of formula E-I is any of those in the tables herein.

In one aspect is a method for treating a disease or disease symptom in a subject comprising administering to the subject an effective amount of a compound of formula F-(I) or pharmaceutical salt thereof: 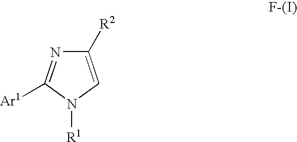
wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently selected from CO₂R³, COAr³, CONR³R⁴, Ar³, CH₂NR³R⁴;
each R³ is independently selected from H, or lower alkyl;
each R⁴ is independently selected from H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each p is independently 0 or 1;
each substituent for Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy; and
   each q is independently 0 or 1.
   In other aspects, the methods are those having any of the formulae herein (including any combinations thereof):
   Wherein,
   each R² is independently CONR³R⁴, Ar³, CH₂NR³R⁴;
   Wherein,
   Ar¹ is aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
   R¹ is Ar²; and
   Ar² is independently aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
   Wherein,
   each R² is independently Ar³; and
   each Ar³ is independently aryl or heteroaryl, each optionally substituted with one or more substituents;
   Wherein,
   each Ar³ is independently heteroaryl, each optionally substituted with one or more substituents;
   Wherein,
   each R² is independently CONR³R⁴; and
   each R⁴ is (CH₂)ₚAr³;
   Wherein,
   each Ar³ is independently aryl or heteroaryl, each optionally substituted with one or more substituents;
   Wherein,
   Ar³ is independently a nitrogen-containing heteroaryl, optionally substituted with one or more substituents;
   Wherein,
   each R² is independently CH₂NR³R⁴; and
   each R⁴ is (CH₂)ₚAr³;
   Wherein,

Ar³ is independently a nitrogen-containing heteroaryl, optionally substituted with one or more substituents.

In one aspect is a compound of formula G-(I) or pharmaceutical salt thereof 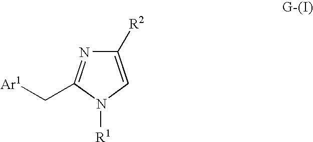
wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently selected from CO₂R³, COAr³, CONR³R⁴, (CH₂)ₘAr³, (CH₂)ₙNR³R⁴ or CH₂OR⁴;
each R³ is independently selected from H, or lower alkyl;
each R⁴ is independently selected from H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each m is independently 0 or 1;
each n is independently 1 or 2;
each p is independently 0 or 1;
each substituent for Ar³ is independently selected from halogen, CN, NO₂, OR⁵, SR⁵, S(O)₂OR⁵,NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(R⁵NR⁵R⁶, NR⁵C(NR⁶)NR⁵R⁶, S(O)₂NR⁵R⁶, R⁷, C(O)R⁷, NR⁶C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy; and
each q is independently 0 or 1.
In other aspects, the compounds are those of any of the formulae herein (including any combinations thereof):
Wherein
Ar¹ is aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar²;
Ar² is independently selected from aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl; and
each R² is independently selected from COAr³, CONR³R⁴, (CH₂)ₘAr³, or (CH₂)ₙNR³R⁴;
Wherein,
Ar¹ is aryl, which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
Ar² is independently aryl, which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl; and
each R² is independently selected from (CH₂)ₘAr³, or (CH₂)ᵣNR³R⁴;
Wherein,
each Ar³ is independently aryl or heteroaryl, each optionally substituted with one or more substituents;
Wherein,
each R⁴ is (CH₂)ₚAr³; and
each Ar³ is independently aryl or heteroaryl, each optionally substituted with one or more substituents;
Wherein,
each R² is independently selected (CH₂)ₙNR³R⁴; and
each R⁴ is (CH₂)ₚAr³;
Wherein,
R¹ is para-chlorophenyl;
Wherein,
Ar¹ is para-fluorophenyl;
Wherein, the compound of formula G-I is a compound delineated in any of the tables herein, or pharmaceutical salt thereof.
In one aspect is a compound of formula H-(I) or pharmaceutical salt thereof 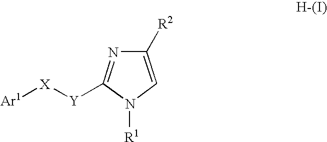
   wherein,
Ar¹ is cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
X is NR³, C(R³)₂, or O;
Y is C═O or lower alkyl;
R¹ is Ar² or lower alkyl optionally substituted with Ar²;
Ar² is independently selected from cycloalkyl, aryl, heterocyclyl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently selected from CO₂R³, COAr³, CONR³R⁴, (CH₂)ₘAr³, CH₂NR³R⁴ or CH₂OR⁴;
each R³ is independently selected from H, or lower alkyl;
each R⁴ is independently selected from H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each m is independently 0 or 1;
each p is independently 0 or 1;
each substituent for Ar³ is independently selected from halogen, CN, NO₂, OR⁵, SR⁵, S(O)₂OR⁵,NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy; and
each q is independently 0 or 1.
In other aspects, the compounds are those of any of the formulae herein (including any combinations thereof):
Wherein,
Ar¹ is aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
X is NR³;
Y is C═O or lower alkyl;
R¹ is Ar²;
Ar² is independently aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl; and
each R² is independently COAr³, CONR³R⁴, (CH₂)ₘAr³, or CH₂NR³R⁴;
Y is C═O; and
Ar² is independently aryl which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
Wherein,
Y is lower alkyl;
R¹ is Ar²;
Ar² is independently aryl or heteroaryl, each of which may be optionally substituted with one or more substituents selected from the group consisting of H, halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
Wherein,
each R² is independently CONR³R⁴ or CH₂NR³R⁴;
Wherein,
each R² is independently (CH₂)ₘAr³;
Wherein,
Ar³ is heteroaryl optionally substituted with one or more substituents;
Wherein,
each R⁴ is independently (CH₂)ₚAr³;
Wherein the compound of formula H-I is a compound delineated in any of the tables herein, or pharmaceutical salt thereof.
One aspect is a compound of formula J-(I) or pharmaceutical salt thereof 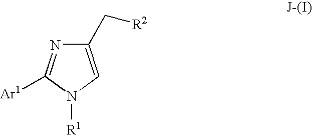
   wherein,
Ar¹ is cycloalkyl, aryl, heterocyclyl, or heteroaryl, each optionally substituted with one or more substituents;
R¹ is Ar² or lower alkyl optionally substituted with Ar²;
each Ar² is independently selected from cycloalkyl, aryl, heterocyclyl, or heteroaryl each optionally substituted with one or more substituents;
each R² is independently (CH₂)ₘCO₂R³, (CH₂)ₘCOAr³, (CH₂)ₘCONR³R⁴, (CH₂)ₘAr³; (CH₂)ₙOR³; (CH₂)ₙAr³ or (CH₂)ₙNR³R⁴;
each R³ is independently selected from H, or lower alkyl;
each R⁴ is independently selected from H, lower alkyl, C(O)OR⁵, C(O)NR⁵R⁶, S(O)₂NR⁵R⁶, C(O)R⁷, S(O)₂)R⁷ or (CH₂)ₚAr³; or
each R³ and R⁴ are taken together with the nitrogen atom to which they are both attached to form a 4-7 membered heterocyclic ring wherein,
   one carbon atom in each heterocyclic ring is optionally a NR⁴, O or S and each heterocyclic ring is optionally substituted with one or more lower alkyl groups;
each Ar³ is independently cycloalkyl, aryl, heterocyclyl, or heteroaryl, each optionally substituted with one or more substituents;
each m is independently 0 or 1;
each n is independently 1 or 2;
each p is independently 0 or 1;
each substituent for Ar³ is independently selected from halogen, CN, NO₂, OR⁵, SR⁵, S(O)₂OR⁵,NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, or S(O)₂R⁷;
each R⁵ is independently selected from hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁶ is independently selected from hydrogen, (CH₂)ₚAr⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently selected from (CH₂)ₚAr⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl; and
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino or 1,2-methylenedioxy.
   Other aspects are those compounds (of any of the formulae herein (including any combinations thereof):
Wherein each R² is independently (CH₂)ₘCO₂R³, (CH₂)ₘCOAr³, (CH₂)ₘCONR³R⁴, (CH₂)ₙAr³ or (CH₂)ₙNR³R⁴;
Wherein,
R¹ is C₁-C₂ alkyl substituted with Ar²; and
Ar² is optionally substituted with one or more substituents;
Wherein,
R¹ is Ar²;
Ar² is optionally substituted with one or more substituents;
Wherein,
R² is (CH₂)ₘC(O)OR³, (CH₂)ₘC(O)Ar³ or (CH₂)ₘC(O)NR³R⁴ and each m is independently 0 or 1; and
each Ar³ is optionally substituted with one or more substituents;
wherein,
R² is (CH₂)ₙNR³R⁴ and n is 1;
Wherein,
R² is (CH₂)ₙNR³R⁴ and n is 2;
Wherein,
R² is (CH₂)ₘAr³ and m is 0; and
Ar³ is optionally substituted with one or more substituents;
Wherein,
R² is (CH₂)ₘAr³ and m is 1; and
Ar³ is optionally substituted with one or more substituents;
Wherein,
each Ar¹, Ar², Ar³ and Ar⁴ is independently selected from cycloalkyl, phenyl, naphthyl, acenaphthyl, indenyl, azulenyl, fluorenyl, anthracenyl, furyl, thienyl, pyridyl, pyrrolyl, oxazolyl, thiazolyl, imidazolyl, pyraxolyl, pyrazolinyl, pyrazolidinyl, isoxazolyl, isotriazolyl, oxadiazolyl, triazolyl, thiadiazolyl, pyridazinyl, pyrimidinyl, pyrazinyl, triazinyl, trithianyl, indolizinyl, indolyl, isoindolyl, 3H-indolyl, indolinyl, benzo-[b]:furanyl, benzo[b]thiophenyl, 1H-indazolyl, benzimidazolyl, benzthiazolyl, purinyl, 4H-quinolizinyl, quinolinyl, tetrahydro-iso quinolinyl, isoquinolinyl, tetrahydro-quinoline, cinnolinyl, phthalazinyl, quinazolinyl, quinoxalinyl, naphthyridinyl, peridinyl, carbazolyl, acridinyl, phenazinyl, phenothiazinyl, or phenoxazinyl, each optionally substituted with one or more substituents;
   Wherein, the compound is of formula J-(I): 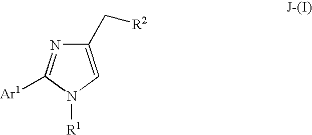
wherein,
Ar¹ is aryl or heteroaryl each optionally substituted with one to three substituents;
R¹ is Ar²;
each Ar² is independently-selected from aryl or heteroaryl each optionally substituted with one to three. substituents;
R² is (CH₂)ₙNR³R⁴ and n is 1 wherein,
each R⁴ is independently selected from H, lower alkyl, C(O)OR⁵, C(O)NR⁵R⁶, S(O)₂NR⁵R⁶, C(O)R⁷, S(O)₂)R⁷ or (CH₂)ₚAr³; or
each R³ and R⁴ are taken together with the nitrogen atom to which they are both attached to form a 4-7 membered heterocyclic ring wherein,
one carbon atom in each heterocyclic ring is optionally a NR⁴, O or S and each heterocyclic ring is optionally substituted with one or two lower alkyl groups;
each p is independently 0 or 1; and
each Ar³ is independently selected from aryl or heteroaryl, each optionally substituted with one to three substituents;
Wherein the compound is of formula J-(I):

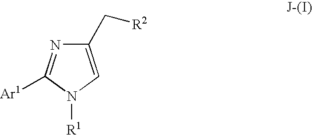

wherein,
Ar¹ is aryl or heteroaryl each optionally substituted with one to three substituents;
R¹ is Ar²;
each Ar² is independently selected from aryl or heteroaryl each optionally substituted with one to three substituents;
R² is (CH₂)ₙNR³R⁴ and n is 2 wherein,
each R⁴ is independently selected from H, lower alkyl, C(O)OR⁵, C(O)NR⁵R⁶, S(O)₂NR⁵R⁶, C(O)R⁷, S(O)₂)R⁷ or (CH₂)ₚAr³; or
each R³ and R⁴ are taken together with the nitrogen atom to which they are both attached to form a 4-7 membered heterocyclic ring wherein,
   one carbon atoms in each heterocyclic ring is optionally a NR⁴, O or S and each heterocyclic ring is optionally substituted with one or two lower alkyl groups;

each p is independently 0 or 1; and
each Ar³ is independently selected from aryl or heteroaryl, each optionally substituted with one to three substituents;
Wherein the compound is of formula J-(I): 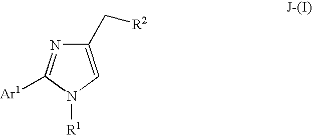
wherein,
Ar¹ is aryl or heteroaryl each optionally substituted with one to three substituents;
R¹ is Ar²;
each Ar² is independently selected from heterocyclyl or heteroaryl each optionally substituted with one to three substituents;
R² is (CH₂)ₘAr³ and m is 0;
each Ar³ is independently selected from aryl or heteroaryl, each optionally substituted with one to three substituents;
each substituent for Ar¹, Ar² and Ar³ is independently selected from halogen, OR⁵, NR⁵R⁶, C₁-C₂ perfluoroalkyl, C₁-C₂ perfluoroalkoxy, 1,2-methylenedioxy;
each R⁵ is independently selected from hydrogen or lower alkyl optionally substituted with one or more substituents selected form halogen, OH, C₁-C₄ alkoxy, NH₂, C₁-C₄ alkylamino, C₁-C₄ dialkylamino;
each R⁶ is independently selected from hydrogen, (CH₂)ₚAr⁴ or lower alkyl optionally substituted with one or more substituents selected form halogen, OH, C₁-C₄ alkoxy, NH₂, C₁-C₄ alkylamino, C₁-C₄ dialkylamino;
each p is independently 0 or 1; and
each Ar⁴ is independently selected from aryl or heteroaryl, each optionally substituted with one to three substituents independently selected halogen, OH, C₁-C₄ alkoxy, NH₂, C₁-C₄ alkylamino, C₁-C₄ dialkylamino, C₁-C₂ perfluoroalkyl, C₁-C₂ perfluoroalkoxy, 1,2-methylenedioxy;
Wherein the compound is of formula J-(I): 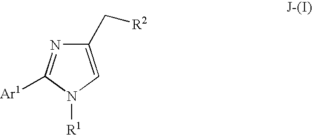
wherein,
Ar¹ is aryl or heteroaryl each optionally substituted with one to three substituents;
R¹ is Ar²;
each Ar² is independently selected from heterocyclyl or heteroaryl each optionally substituted with one to three substituents;
R² is (CH₂)ₘAr³ and m is 1;
each Ar³ is independently selected from aryl or heteroaryl, each optionally substituted with one to three substituents;
each substituent for Ar¹, Ar² and Ar³ is independently selected from halogen, OR⁵, NR⁵R⁶, C₁-C₂ perfluoroalkyl, C₁-C₂ perfluoroalkoxy, 1,2-methylenedioxy;
each R⁵ is independently selected from hydrogen or lower alkyl optionally substituted with one or more substituents selected form halogen, OH, C₁-C₄ alkoxy, NH₂, C₁-C₄ alkylamino, C₁-C₄ dialkylamino;
each R⁶ is independently selected from hydrogen, (CH₂)ₚAr⁴ or lower alkyl optionally substituted with one or more substituents selected form halogen, OH, C₁-C₄ alkoxy, NH₂, C₁-C₄ alkylamino, C₁-C₄ dialkylamino;
each p is independently 0 or 1, and
each Ar⁴ is independently selected from aryl or heteroaryl, each optionally substituted with one to three substituents independently selected halogen, OH, C₁-C₄ alkoxy, NH₂, C₁-C₄ alkylamino, C₁-C₄ dialkylamino, C₁-C₂ perfluoroalkyl, C₁-C₂ perfluoroalkoxy, 1,2-methylenedioxy;
Wherein the compound is of formula J-(I):

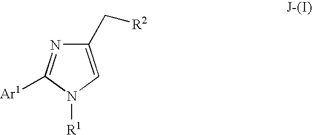

wherein,
Ar¹ is phenyl substituted with one to three substituents;
R¹ is Ar² and Ar² phenyl substituted with one to three substituents;
R² is (CH₂)ₙNR³R⁴ and n is 1;

each R³ is independently selected from H or lower alkyl;
each R⁴ is (CH₂)ₚAr³;
each p is independently 0 or 1;
   each Ar³ is independently selected from aryl or heteroaryl, each optionally substituted with one to three substituents;
each substituent for Ar¹, Ar² and Ar³ is independently selected from halogen, OR⁵, NR⁵R⁶, C₁-C₂ perfluoroalkyl, C₁-C₂ perfluoroalkoxy, 1,2-methylenedioxy;
each R⁵ is independently selected from hydrogen or lower alkyl optionally substituted with one or more substituents selected form halogen, OH, C₁-C₄ alkoxy, NH₂, C₁-C₄ alkylamino, C₁-C₄ dialkylamino;
each R⁶ is independently selected from hydrogen, (CH₂)ₚAr⁴ or lower alkyl optionally substituted with one or more substituents selected form halogen, OH, C₁-C₄ alkoxy, NH₂, C₁-C₄ alkylamino, C₁-C₄ dialkylamino; and
each Ar⁴ is independently selected from aryl or heteroaryl, each optionally substituted with one to three substituents independently selected halogen, OH, C₁-C₄ alkoxy, NH₂, C₁-C₄ alkylamino, C₁-C₄ dialkylamino, C₁-C₂ perfluoroalkyl, C₁-C₂ perfluoroalkoxy, 1,2-methylenedioxy;
Wherein the compound is of formula J-(I): 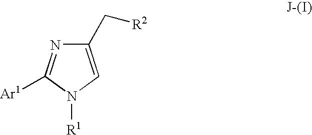
wherein,
Ar¹ is phenyl substituted with one to three substituents;
R¹ is Ar² and Ar² phenyl substituted with one to three substituents;
R² is (CH₂)ₙNR³R⁴ and n is 1;
each R³ and R⁴ are taken together with the nitrogen atom to which they are both attached to form a 4-7 membered heterocyclic ring wherein,
one carbon atoms in each heterocyclic ring is optionally a NR⁴, O or S and each heterocyclic ring is optionally substituted with one or two lower alkyl groups;
each substituent for Ar¹ and Ar² is independently selected from halogen, OR⁵, NR⁵R⁶, C₁-C₂ perfluoroalkyl, C₁-C₂ perfluoroalkoxy, 1,2-methylenedioxy
each R⁵ is independently selected from hydrogen or lower alkyl optionally substituted with one or more substituents selected form halogen, OH, C₁-C₄ alkoxy, NH₂, C₁-C₄ alkylamino, C₁-C₄ dialkylamino;
each R⁶ is independently selected from hydrogen, (CH₂)ₚAr⁴ or lower alkyl optionally substituted with one or more substituents selected form halogen, OH, C₁-C₄ alkoxy, NH₂, C₁-C₄ alkylamino, C₁-C₄ dialkylamino;
each p is independently 0 or 1; and
each Ar⁴ is independently selected from aryl or heteroaryl, each optionally substituted with one to three substituents independently selected halogen, OH, C₁-C₄ alkoxy, NH₂, C₁-C₄ alkylamino, C₁-C₄ dialkylamino, C₁-C₂ perfluoroalkyl, C₁-C₂ perfluoroalkoxy, 1,2-methylenedioxy;
Wherein compound is of formula J-(I): 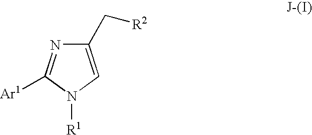
wherein,
Ar¹ is phenyl substituted with one to three substituents;
R¹ is Ar² and Ar² phenyl substituted with one to three substituents;
R² is (CH₂)ₘAr³ and m is 0;
each Ar³ is benzimidazol-2-yl optionally substituted with one to three substituents;
each substituent for Ar¹, Ar² and Ar³ is each independently selected from halogen, OR⁵, NR⁵R⁶, C₁-C₂ perfluoroalkyl, C₁-C₂ perfluoroalkoxy, 1,2-methylenedioxy;
each R⁵ is independently selected from hydrogen or lower alkyl optionally substituted with one or more substituents selected form halogen, OH, C₁-C₄ alkoxy, NH₂, C₁-C₄ alkylamino, C₁-C₄ dialkylamino;
each R⁶ is independently selected from hydrogen, (CH₂)ₚAr⁴ or lower alkyl optionally substituted with one or more substituents selected form halogen, OH, C₁-C₄ alkoxy, NH₂, C₁-C₄ alkylamino, C₁-C₄ dialkylamino;
each p is independently 0 or 1; and
each Ar⁴ is independently selected from aryl or heteroaryl, each optionally substituted with one to three substituents independently selected halogen, OH, C₁-C₄ alkoxy, NH₂, C₁-C₄ alkylamino, C₁-C₄ dialkylamino, C₁-C₂ perfluoroalkyl, C₁-C₂ perfluoroalkoxy, 1,2-methylenedioxy; or
   Wherein the compound of formula J-(I) is any of those in Table J-1 herein.

Another aspect is a method of treating a Cav1 calcium channel mediated disease or disease symptom in a subject comprising administering to the subject an effective amount of a compound, or pharmaceutical salt, (or composition thereof) of any of the formulae herein.

Another aspect is a method of modulating (e.g., inhibiting, agonism, antagonism) calcium channel activity including contacting a compound, or pharmaceutical salt thereof, of any of the formulae herein (or composition thereof) with a calcium channel.

In the methods herein, the calcium channel can be Caᵥ1 (e.g., Caᵥ1.2 or Caᵥ1.3). The Caᵥ1 calcium channel mediated disease or disease symptom can be a nervous system disease or disease symptom or can be a cardiovascular disease or disease symptom.

Another aspect is a method of treating a mediated disease or disease symptom in a subject comprising administering to the subject an effective amount of a compound, or pharmaceutical salt, (or composition thereof) of any of the formulae herein. The disease or disease symptom is angina, hypertension, congestive heart failure, myocardial ischemia, atrial fibrillation, diabetes mellitus, urinary incontinence, overactive bladder, pulmonary disease, cognitive function, or a nervous system disorder;

Wherein, the disease or disease symptom is modulated by calcium channel Cav1; Wherein the disease or disease symptom is modulated by calcium channel Cav1.2 or Cav1.3; Wherein the disease or disease symptom is angina, hypertension, congestive heart failure, myocardial ischemia, arrhythmia, diabetes, urinary incontinence, stroke, pain, traumatic brain injury, or a neuronal disorder.

Another aspect is a composition including a compound of any of the formulae herein, or pharmaceutically acceptable salt thereof and a pharmaceutically acceptable carrier. The composition can further include an additional therapeutic agent.

Another aspect is a method of making a compound of any of the formulae herein, including reacting an intermediate delineated herein with a reagent to provide a compound of any of the formulae herein as defined herein.

Another aspect-is a method of modulating (e.g., inhibiting, antagonism, agonism) calcium channel activity in a subject in need thereof comprising administering to the subject an effective amount of a compound of any of the formulae herein, or pharmaceutically acceptable salt thereof, or composition thereof.

In other aspects, the invention relates to a composition comprising a compound of any of the formulae herein, an additional therapeutic agent, and a pharmaceutically acceptable carrier. The additional therapeutic agent can be a cardiovascular disease agent and/or a nervous system disease agent. A nervous system disease agent refers to a peripheral nervous system (PNS) disease agent and/or a central nervous system (CNS) disease agent.

Yet another aspect of this invention relates to a method of treating a subject (e.g., mammal, human, horse, dog, cat) having a disease or disease symptom (including, but not limited to angina, hypertension, congestive heart failure, myocardial ischemia, atrial fibrillation, diabetes mellitus, urinary incontinence, overactive bladder, pulmonary disease, cognitive function, or a nervous system disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Yet another aspect of this invention relates to a method of treating a subject (e.g., mammal, human, horse, dog, cat) having an ion channel mediated disease or disease symptom (including, but not limited to angina, hypertension, congestive heart failure, myocardial ischemia, atrial fibrillation, diabetes mellitus, urinary incontinence, overactive bladder, pulmonary disease, cognitive function, or a nervous system disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The invention also relates to a method of making a compound described herein, the method including any reactions or reagents as delineated in the schemes or examples herein. Alternatively, the method includes taking any one of the intermediate compounds described herein and reacting it with one or chemical reagents in one or more steps to produce a compound described herein.

Also within the scope of this invention is a packaged product. The packaged product includes a container, one of the aforementioned compounds in the container, and a legend (e.g., a label or an insert) associated with the container and indicating administration of the compound for treating a disorder associated with ion channel modulation.

In other embodiments, the compounds, compositions, and methods delineated herein are any of the compounds of the Tables herein or methods including them.

The details of one or more embodiments of the invention are set forth in the accompanying drawings and the description below. Other features, objects, and advantages of the invention will be apparent from the description and from the claims.

### DETAILED DESCRIPTION

As used herein, the term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₅ indicates that the group may have from 1 to 5 (inclusive) carbon atoms in it. The term “lower alkyl” refers to a C₁-C₆ alkyl chain. The term “arylalkyl” refers to a moiety in which an alkyl hydrogen atom is replaced by an aryl group.

The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene.

The term “cycloalkyl” as employed herein includes saturated and partially unsaturated cyclic hydrocarbon groups having 3 to 12 carbons, preferably 3 to 8 carbons, and more preferably 3 to 6 carbon.

The term “aryl” refers to a 6-membered monocyclic or 10- to 14-membered multicyclic aromatic hydrocarbon ring system wherein 0, 1, 2, 3, or 4 atoms of each ring may be substituted by a substituent. Examples of aryl groups include phenyl, naphthyl and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation halogen, CN, NO₂, OR⁵, SR⁵, S(O)₂OR⁵, NR⁵R⁶, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷. Each R⁵ is independently hydrogen, C₁-C₄ alkyl or C₃-C₆ cycloalkyl. Each R⁶ is independently hydrogen, C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each R⁷ is independently C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each C₃-C₆ cycloalkyl, aryl, heterocyclyl, heteroaryl and C₁-C₄ alkyl in each R⁵, R⁶ and R⁷ can optionally be substituted with halogen, CN, C₁-C₄ alkyl; OH, C₁-C₄ alkoxy, NH₂, C₁-C₄ alkylamino, C₁-C₄ dialkylamino, C₁-C₂ perfluoroalkyl, C₁-C₂ perfluoroalkoxy, or 1,2-methylenedioxy.

In one aspect, the substituents on a group are independently, hydrogen, hydroxyl, halogen, nitro, SO₃H, trifluoromethyl, trifluoromethoxy, alkyl (C1-C6 straight or branched), alkoxy (C1-C6 straight or branched), O-benzyl, O-phenyl, phenyl, 1,2-methylenedioxy, carboxyl, morpholinyl, piperidinyl, amino or OC(O)NR⁵R⁶. Each R⁵ and R⁶ is as described above.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg. Effective doses will also vary depending on route of administration, as well as the possibility of co-usage with other agents.

Representative compounds useful in the compositions and methods are delineated herein:

LENGTHY TABLE REFERENCED HERE
US20070281937A1-20071206-T00001
Please refer to the end of the specification for access instructions.

LENGTHY TABLE REFERENCED HERE
US20070281937A1-20071206-T00002
Please refer to the end of the specification for access instructions.

LENGTHY TABLE REFERENCED HERE
US20070281937A1-20071206-T00003
Please refer to the end of the specification for access instructions.

LENGTHY TABLE REFERENCED HERE
US20070281937A1-20071206-T00004
Please refer to the end of the specification for access instructions.

LENGTHY TABLE REFERENCED HERE
US20070281937A1-20071206-T00005
Please refer to the end of the specification for access instructions.

LENGTHY TABLE REFERENCED HERE
US20070281937A1-20071206-T00006
Please refer to the end of the specification for access instructions.

LENGTHY TABLE REFERENCED HERE
US20070281937A1-20071206-T00007
Please refer to the end of the specification for access instructions.

LENGTHY TABLE REFERENCED HERE
US20070281937A1-20071206-T00008
Please refer to the end of the specification for access instructions.

LENGTHY TABLE REFERENCED HERE
US20070281937A1-20071206-T00009
Please refer to the end of the specification for access instructions.

LENGTHY TABLE REFERENCED HERE
US20070281937A1-20071206-T00010
Please refer to the end of the specification for access instructions.

LENGTHY TABLE REFERENCED HERE
US20070281937A1-20071206-T00011
Please refer to the end of the specification for access instructions.

LENGTHY TABLE REFERENCED HERE
US20070281937A1-20071206-T00012
Please refer to the end of the specification for access instructions.

LENGTHY TABLE REFERENCED HERE
US20070281937A1-20071206-T00013
Please refer to the end of the specification for access instructions.

LENGTHY TABLE REFERENCED HERE
US20070281937A1-20071206-T00015
Please refer to the end of the specification for access instructions.

LENGTHY TABLE REFERENCED HERE
US20070281937A1-20071206-T00016
Please refer to the end of the specification for access instructions.

LENGTHY TABLE REFERENCED HERE
US20070281937A1-20071206-T00017
Please refer to the end of the specification for access instructions.

LENGTHY TABLE REFERENCED HERE
US20070281937A1-20071206-T00018
Please refer to the end of the specification for access instructions.

LENGTHY TABLE REFERENCED HERE
US20070281937A1-20071206-T00019
Please refer to the end of the specification for access instructions.

LENGTHY TABLE REFERENCED HERE
US20070281937A1-20071206-T00020
Please refer to the end of the specification for access instructions.

LENGTHY TABLE REFERENCED HERE
US20070281937A1-20071206-T00021
Please refer to the end of the specification for access instructions.

LENGTHY TABLE REFERENCED HERE
US20070281937A1-20071206-T00022
Please refer to the end of the specification for access instructions.

LENGTHY TABLE REFERENCED HERE
US20070281937A1-20071206-T00023
Please refer to the end of the specification for access instructions.

LENGTHY TABLE REFERENCED HERE
US20070281937A1-20071206-T00024
Please refer to the end of the specification for access instructions.

LENGTHY TABLE REFERENCED HERE
US20070281937A1-20071206-T00025
Please refer to the end of the specification for access instructions.

LENGTHY TABLE REFERENCED HERE
US20070281937A1-20071206-T00026
Please refer to the end of the specification for access instructions.

LENGTHY TABLE REFERENCED HERE
US20070281937A1-20071206-T00027
Please refer to the end of the specification for access instructions.

LENGTHY TABLE REFERENCED HERE
US20070281937A1-20071206-T00028
Please refer to the end of the specification for access instructions.

LENGTHY TABLE REFERENCED HERE
US20070281937A1-20071206-T00029
Please refer to the end of the specification for access instructions.

LENGTHY TABLE REFERENCED HERE
US20070281937A1-20071206-T00030
Please refer to the end of the specification for access instructions.

LENGTHY TABLE REFERENCED HERE
US20070281937A1-20071206-T00031
Please refer to the end of the specification for access instructions.

LENGTHY TABLE REFERENCED HERE
US20070281937A1-20071206-T00032
Please refer to the end of the specification for access instructions.

LENGTHY TABLE REFERENCED HERE
US20070281937A1-20071206-T00033
Please refer to the end of the specification for access instructions.

LENGTHY TABLE REFERENCED HERE
US20070281937A1-20071206-T00034
Please refer to the end of the specification for access instructions.

LENGTHY TABLE REFERENCED HERE
US20070281937A1-20071206-T00035
Please refer to the end of the specification for access instructions.

LENGTHY TABLE REFERENCED HERE
US20070281937A1-20071206-T00036
Please refer to the end of the specification for access instructions.

LENGTHY TABLE REFERENCED HERE
US20070281937A1-20071206-T00037
Please refer to the end of the specification for access instructions.

Ion channel-modulating compounds can be identified through both in vitro (e.g., cell and non-cell based) and in vivo methods. Representative examples of these methods are described in the Examples herein.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The compounds delineated herein can be synthesized using conventional methods, as illustrated in the schemes herein. In the schemes herein, unless expressly to the contrary, variables in chemical formulae are as defined in other formulae herein. For example, Ar¹, Ar³, R¹, R², R³ and R⁴ in the schemes are defined as in any of the formulae herein, except where defined otherwise in the schemes.

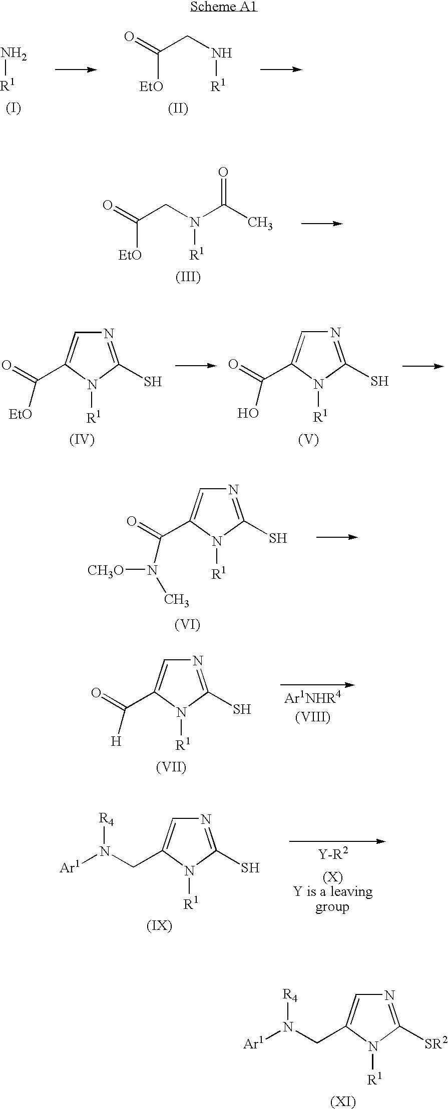

Treatment of amine (I) under basic conditions (e.g., sodium acetate) with ethyl bromoacetate in solvent provides amino acid ester (II). Treatment of (II) in solvent with acetyl chloride provides (III). The imidazole (IV) is produced when (III) is treated with ethyl formate in solvent under basic conditions. Saponification of ester (IV) under basic conditions gives imidazole (V). Treatment of (V) with N,O-dimethylhydroxylamine under amide bond forming conditions (e.g., 1-ethyl-3-(3-dimethylaminopropyl)-carbodiimide hydrochloride) in solvent gives amide (VI). Treatment of amide (VI) under reducing conditions (e.g., lithium aluminum hydride) in solvent provides aldehyde (VII). Treatment of (VII) with amine (VIII) under reducing conditions (e.g., lithium aluminum hydride, THF) provides (IX). Treatment of (IX) with (X) gives the desired compound (XI).

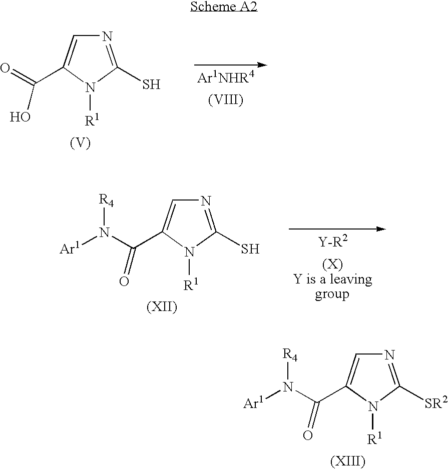

Treatment of carboxylic acid (V) with amine (VIII) under amide bond forming conditions (e.g., 1-ethyl-3-(3-dimethylaminopropyl)-carbodiimide hydrochloride) in solvent gives amide (XII). Treatment of (XII) with (X) under basic conditions (e.g., K₂CO₃) in solvent provides (XIII).

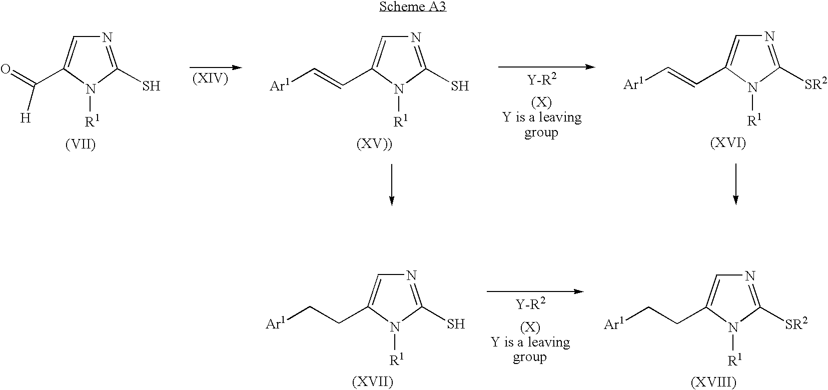

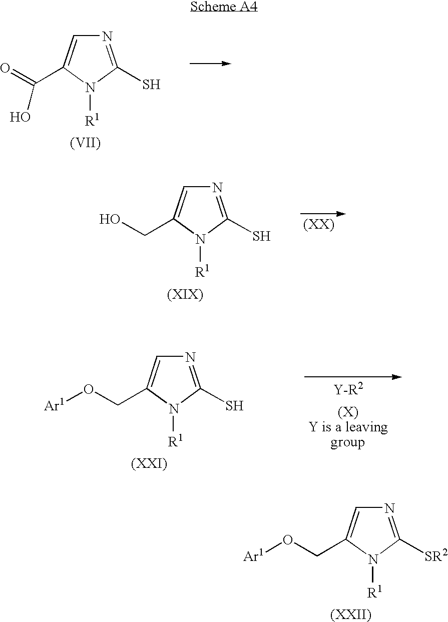

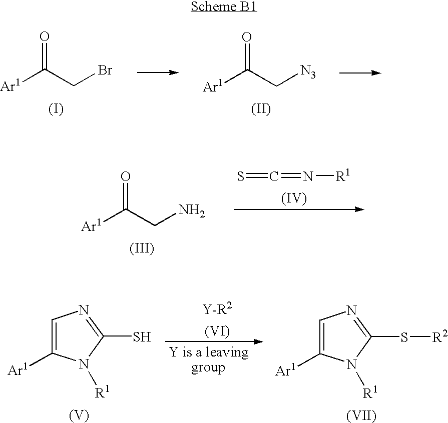

Treatment of the bromomethyl ketone (I) in solvent, such as DMSO, with sodium azide provides the azidomethyl ketone (II). Treatment of (II) with reducing conditions, such as palladium on carbon in aqueous HCl and H₂ atmosphere, provides the amine (III). The reaction of (III) and isothiocyanate (IV) under basic conditions, such as sodium hydrogencarbonate, in a solvent, such as ethanol, provides thioimidazole (V). The reaction of (V) and (VI) under basic condition, such as potassium carbonate, provides imidazole (VII).

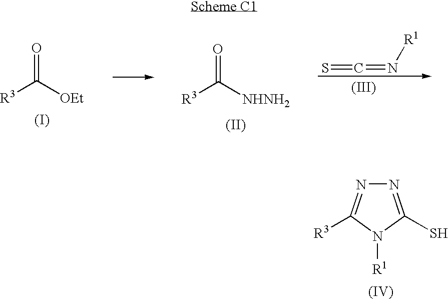

Treatment of ethyl ester (I) with hydrazine in solvent (e.g., ethanol) provides hydrazide (II). Treatment of (II) with thioisocyanate (III) under aqueous basic conditions gives triazole thiol (IV).

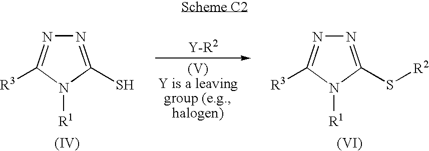

Treatment of (IV) with (V) under basic conditions (e.g., K₂CO₃ in acetone) gives (VI).

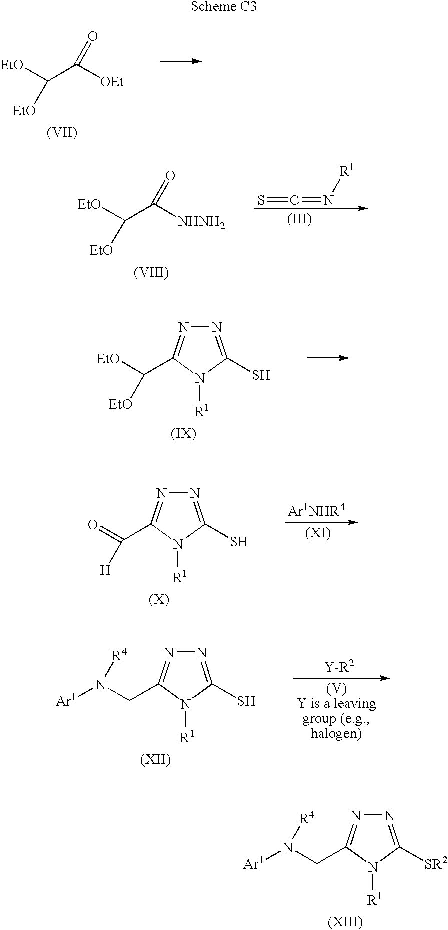

Treatment of ethyl diethoxy acetate (VII) with hydrazine in solvent (e.g., ethanol) provides hydrazide (VIII). Treatment of (VIII) with thioisocyanate (III) aqueous basic conditions gives triazole (IX) which turn provides aldehyde (X) upon treatment with aqueous acidic conditions. Treatment of (X) with (V) under basic conditions (e.g., K₂CO₃ in acetone) provides (XIII).

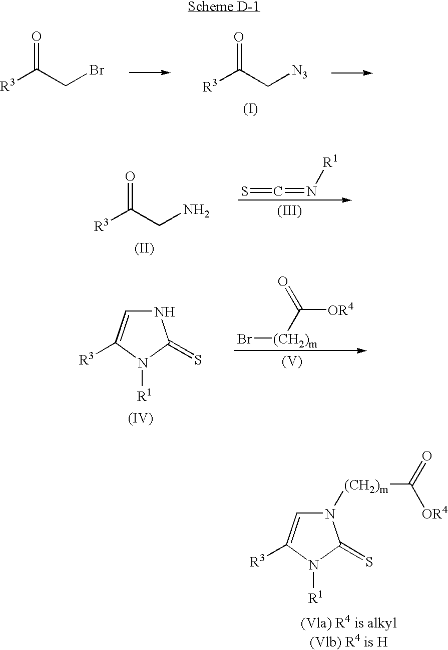

Treatment of the bromomethyl compound with sodium azide provides azidomethyl compound (I). Treatment of (I) under reducing conditions, such as palladium on carbon in aqueous HCl and H₂ atmosphere, provides amine (II). Treatment of (II) with isothiocyanate (III) provides imidazole (IV). N-Alkylated imidazole (Vla) is produced from the reaction of (IV) with 3-bromo-proprionate or 4-bromo-butyrate (V). Saponification of the ester (VIa) gives the carboxylic acid (VIb).

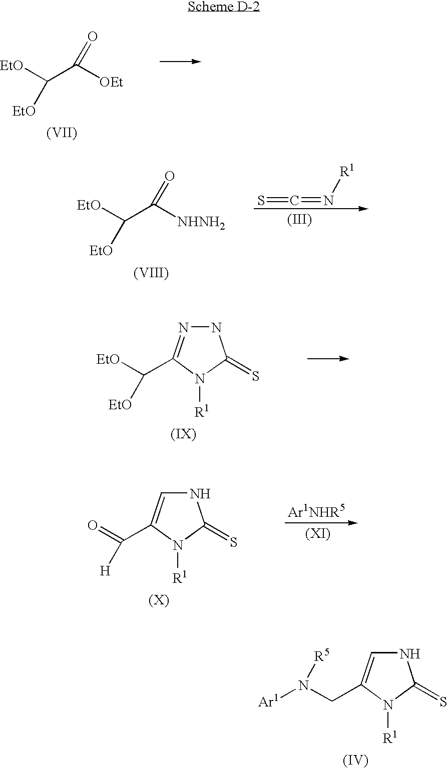

Alternatively, imidazole (IV) is prepared by the following sequence. Treatment of ethyl diethoxy acetate (VII) with hydrazine in solvent (e.g., ethanol) provides hydrazide (VIII). Treatment of (VIII) with thioisocyanate (III) under aqueous basic conditions gives imidazole (IX) which in turn provides aldehyde (X) under aqueous acidic conditions. Reductive amination of (X) and amine (XI) provides (IV).

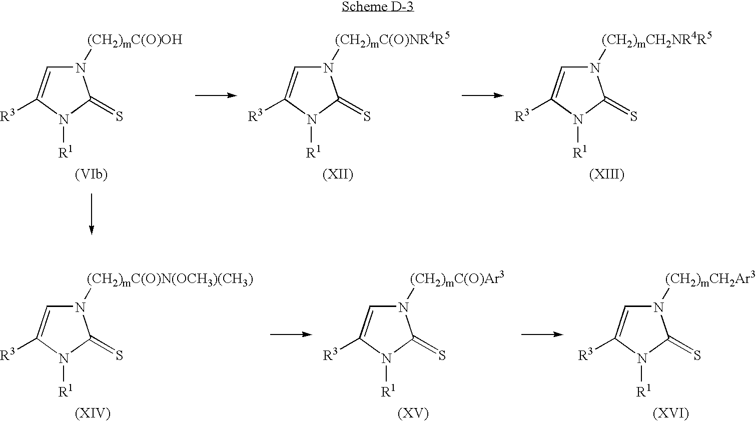

The reaction of carboxylic acid (VIb) with the appropriately substituted amine under standard coupling procedures provides the desired amide (XII). Reduction of the amide under common reducing conditions (e.g., diborane or lithium aluminum hydride) provides the corresponding amine (XIII). Alternatively, treatment of (VIb) with Weinreb's reagent provides the amide (XIV). Treatment of the amide (XIV) under standard conditions with an organometallic reagent (e.g., aryl lithium or aryl magnesium halide) provides the ketone (XV). Reduction of the ketone under a variety conditions affords the desired product (XVI).

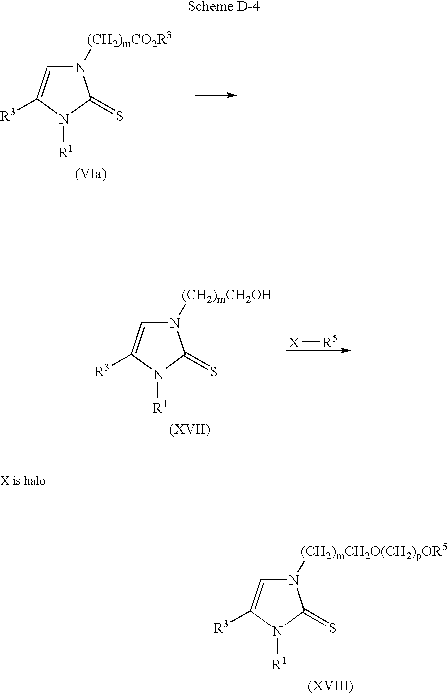

Treatment of ester (Via) under standard reducing conditions (e.g., lithium aluminum hydride) gives alcohol (XVII). Treatment of (XVII) under standard ether forming conditions (e.g., NaH, benzylbromide) gives (XVIII).

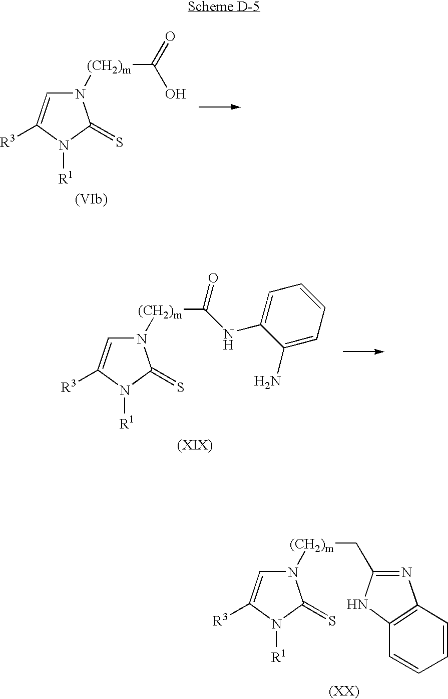

An alternative route to obtain heteroaryl derivatives is to react the activated acid of (VIb) with the appropriate substrate followed by cyclization to provide the desired product. For example as depicted in Scheme D-5, reaction of the activated acid of (VIb) with benzene-1,2-diamine provides the intermediate amide (XIX), which is cyclized to afford the benzimidazole derivative (XX).

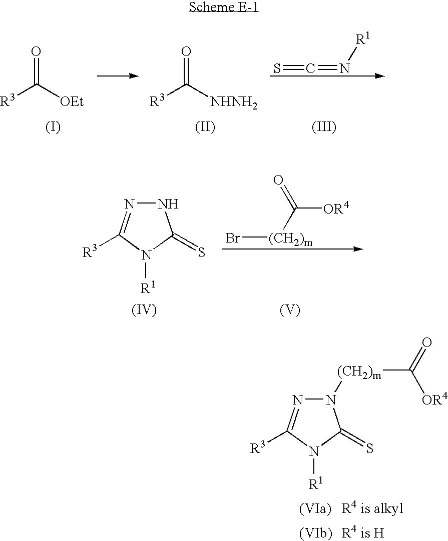

Treatment of ethyl ester (I) with hydrazine in solvent (e.g., ethanol) provides hydrazide (II). Treatment of (II) with thioisocyanate (III) under aqueous basic conditions gives triazole thione (IV). N-Alkylated triazole (VIa) is produced from the reaction of (IV) with 3-bromo-proprionate or 4-bromo-butyrate (V). Saponification of the ester (Via) gives the carboxylic acid (VIb).

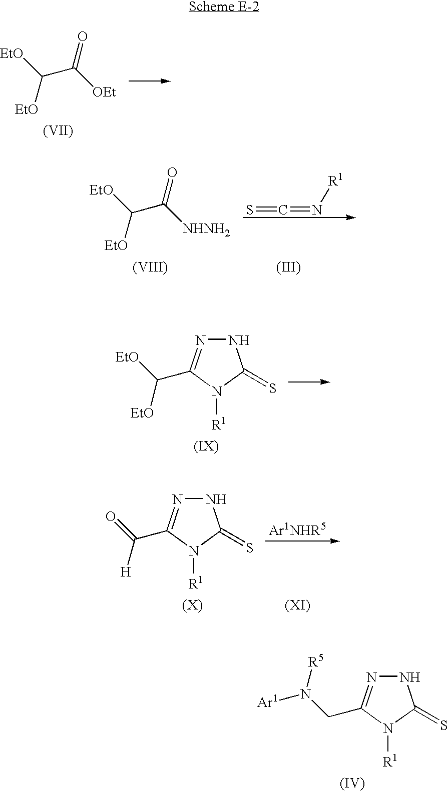

Alternatively, triazole (IV) is prepared by the following sequence. Treatment of ethyl diethoxy acetate (VII) with hydrazine in solvent (e.g., ethanol) provides hydrazide (VIII). Treatment of (VIII) with thioisocyanate (III) under aqueous basic conditions gives triazole (IX) which in turn provides aldehyde (X) under aqueous acidic conditions. Reductive amination of (X) and amine (XI) provides (IV).

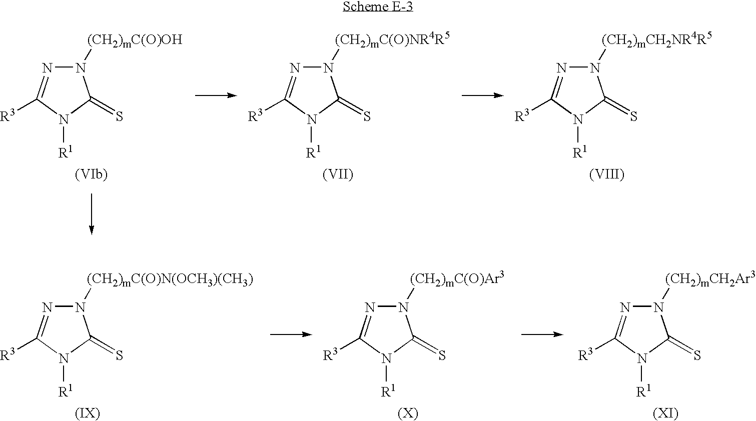

The reaction of carboxylic acid (VIb) with the appropriately substituted amine under standard coupling procedures provides the desired amide (VII). Reduction of the amide under common reducing conditions (e.g., diborane or lithium aluminum hydride) provides the corresponding amine (VIII). Alternatively, treatment of (VIb) with Weinreb's reagent provides the amide (IX). Treatment of the amide (IX) under standard conditions with an organometallic reagent (e.g., aryl lithium or aryl magnesium halide) provides the ketone (X). Reduction of the ketone under a variety conditions affords the desired product (XI).

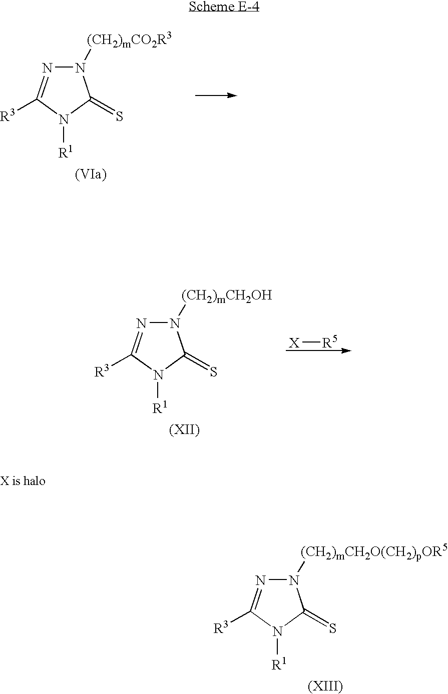

Treatment of ester (VIa) under standard reducing conditions (e.g., lithium aluminum hydride) gives alcohol (XII). Treatment of (XII) under standard ether forming conditions (e.g., NaH, benzylbromide) gives (XIII).

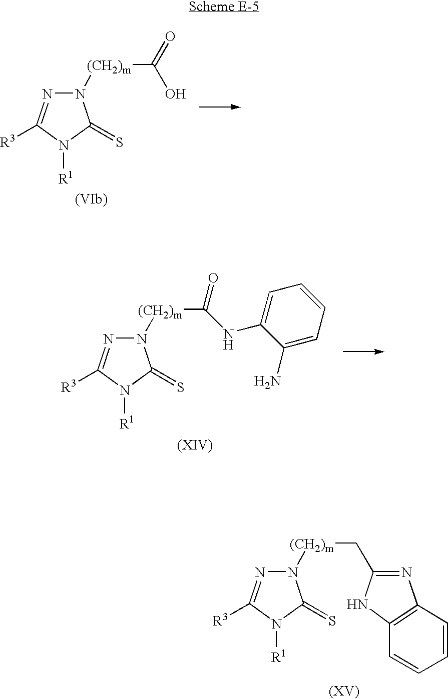

An alternative route to obtain heteroaryl derivatives is to react the activated acid of (VIb) with the appropriate substrate followed by cyclization to provide the desired product. For example as depicted in Scheme E-5, reaction of the activated acid of (VIb) with benzene-1,2-diamine provides the intermediate amide (XIV), which is cyclized to afford the benzimidazole derivative (XV).

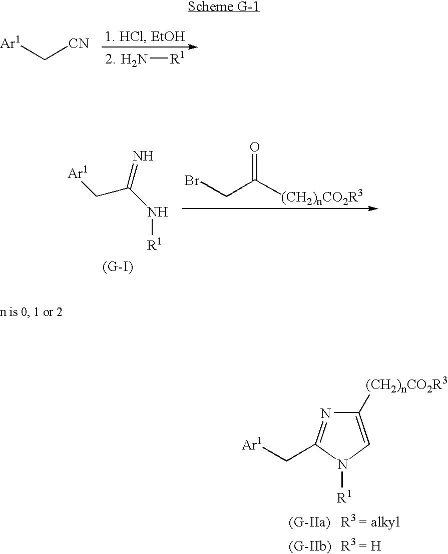

Treatment of an aryl nitrile with an alcohol under acidic conditions provides the alkoxy imidate intermediate, which is treated with the appropriate substituted amine under catalytic conditions (e.g., ethanolic HCl; CuCl; Ln(III) ions) to provide the substituted amidine (G-I). Treatment of amidine (G-I) with a bromopyruvate or a 4-bromo-3-oxo-butyrate or a 5-bromo-oxo-pentanoate under basic conditions provides the imdiazole ester (G-IIa), which is hydrolyzed to provide the corresponding acid derivative (G-IIb).

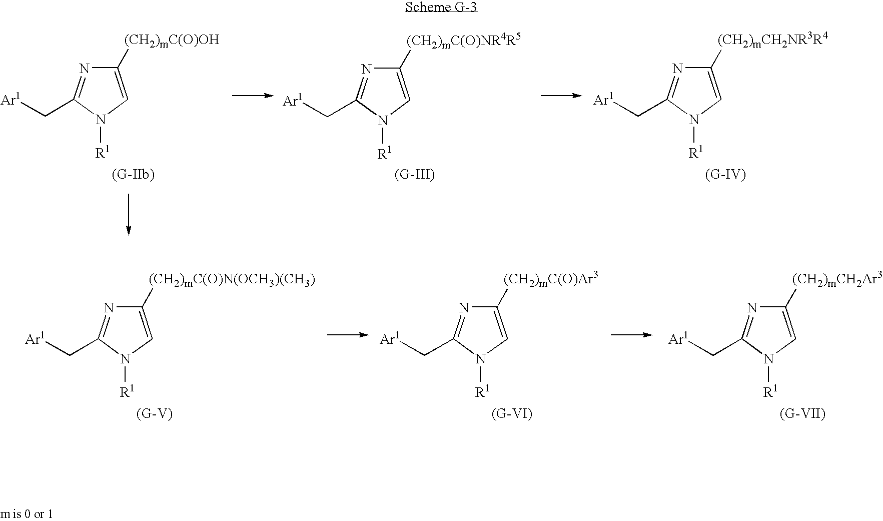

Reaction of the acid (G-IIb) with the appropriately substituted amine under standard coupling procedures provides the desired amide (G-III). Reduction of the amide with common reducing agents such as diborane or lithium aluminum hydride provides the corresponding amine (G-IV). Alternatively treatment of the acid (G-IIb) with Weinreb's reagent provides amide (G-V). Treatment of the amide under standard condition with an organometallic reagent (ex. aryl lithium or aryl magnesium halide) provides the ketone (G-VI). Reduction of the ketone under a variety of conditions affords the desired product (G-VII).

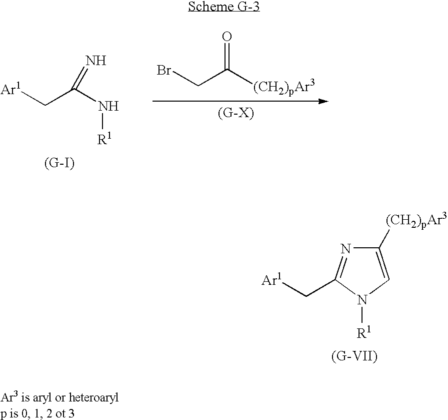

Alternatively treatment of amidine (G-I) with (G-X) provides the desired imidazole (G-VII).

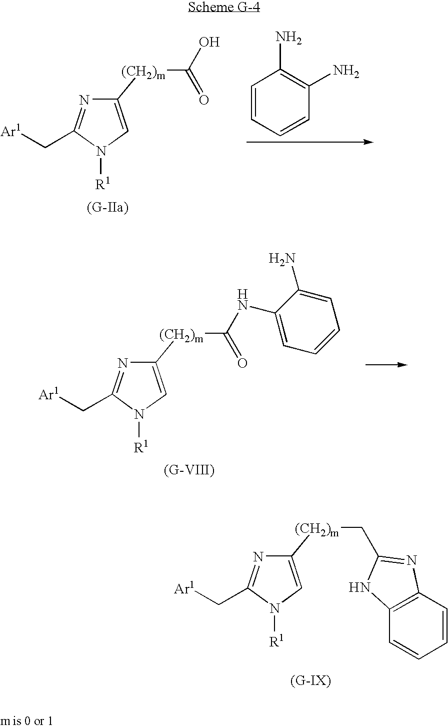

An alternative route to obtain heteroaryl derivatives is to react the activated acid of (G-IIb) with the appropriate substrate followed by cyclization to provide the desired product. For example as depicted in Scheme G-4, reaction of the activated acid of (G-IIb) with benzene-1,2-diamine provides the intermediate amide (G-VIII), which is cyclized to afford the benzimidazole derivative (G-IX).

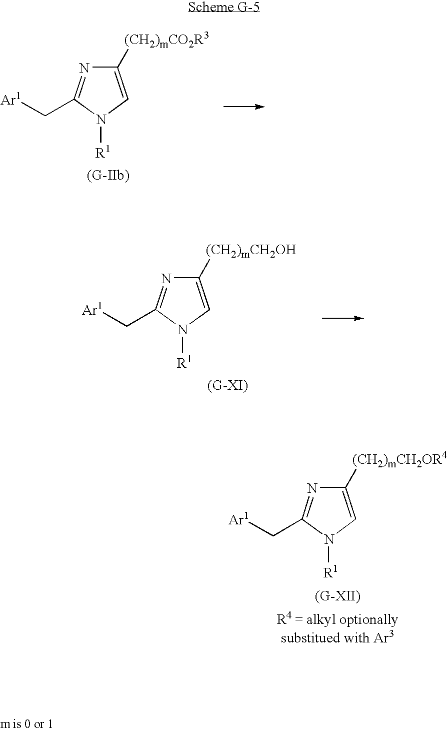

Treatment of carboxylic acid (G-IIb) under standard reducing conditions (e.g., lithium aluminum hydride) gives (G-XI). Treatment of (G-XI) under standard ether forming conditions (e.g., NaH, halo-R⁴) gives (G-XII).

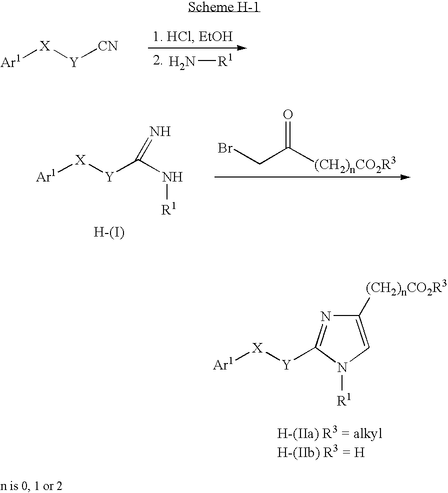

Treatment of an aryl nitrile with an alcohol under acidic conditions provides the alkoxy imidate intermediate, which is treated with the appropriate substituted amine under catalytic conditions (e.g., ethanolic HCl; CuCl; Ln(III) ions) to provide the substituted amidine H-(I). Treatment of amidine H-(I) with a bromopyruvate or a 4-bromo-3-oxo-butyrate or a 5-bromo-oxo-pentanoate under basic conditions provides the imdiazole ester H-(IIa), which is hydrolyzed to provide the corresponding acid derivative H-(IIb).

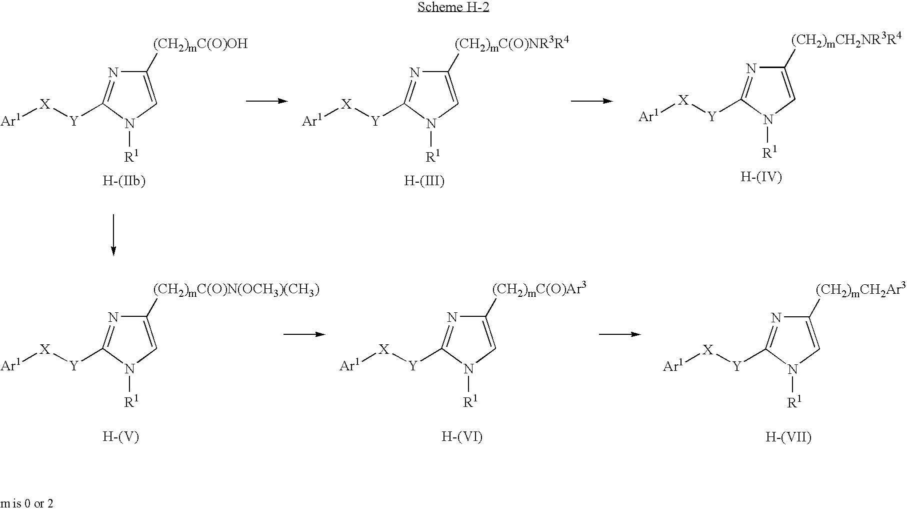

Reaction of the acid H-(IIb) with the appropriately substituted amine under standard coupling procedures provides the desired amide H-(III). Reduction of the amide with common reducing agents such as diborane or lithium aluminum hydride provides the corresponding amine H-(IV). Alternatively treatment of the acid H-(IIb) with Weinreb's reagent provides amide H-(V). Treatment of the amide under standard condition with an organometallic reagent (ex. aryl lithium or aryl magnesium halide) provides the ketone H-(VI). Reduction of the ketone under a variety of conditions affords the desired product H-(VII).

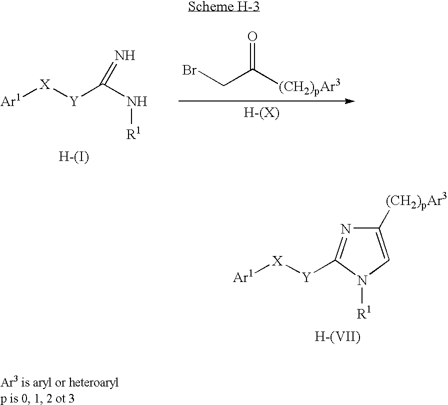

Alternatively treatment of amidine H-(I) with H-(X) provides the desired imidazole H-(VII).

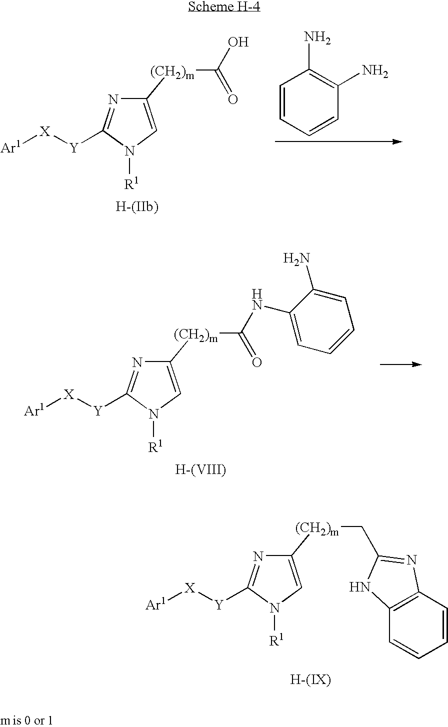

An alternative route to obtain heteroaryl derivatives is to react the activated acid of H-(IIb) with the appropriate substrate followed by cyclization to provide the desired product. For example as depicted in Scheme 4, reaction of the activated acid of H-(IIb) with benzene-1,2-diamine provides the intermediate amide H-(VIII), which is cyclized to afford the benzimidazole derivative H-(IX).

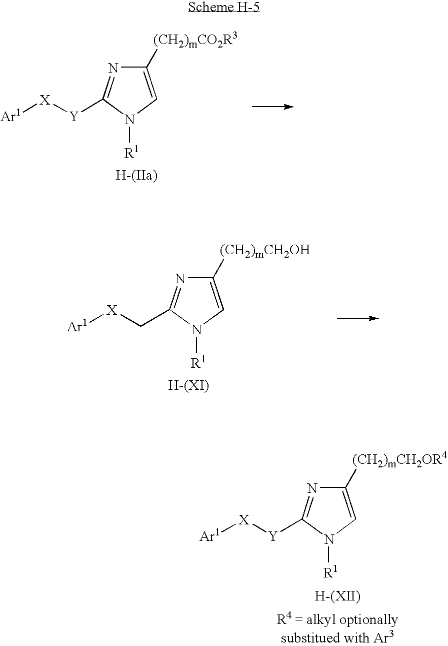

Treatment of carboxylic acid (H-IIa under standard reducing conditions (e.g., lithium conditions (e.g., NaH, halo-R⁴) reactions gives H-(XII).

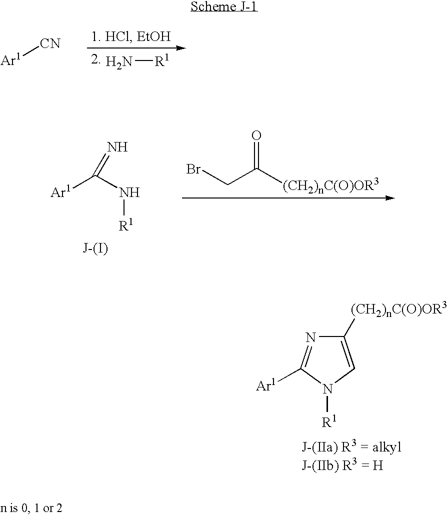

Treatment of an aryl nitrile with an alcohol under acidic conditions provides the alkoxy imidate intermediate, which is treated with the appropriate substituted amine under catalytic conditions (e.g., ethanolic HCl; CuCl; Ln(III) ions) to provide the substituted amidine J-(I). Treatment of amidine J-(I) with a bromopyruvate, a 4-bromo-3-oxo-butyrate, a 5-bromo-4-oxo-pentanoate or a 6-bromo-5-oxo-hexanoate under basic conditions provides the corresponding imidiazole ester J-(IIa), which is hydrolyzed to provide the corresponding acid derivative J-(IIb).

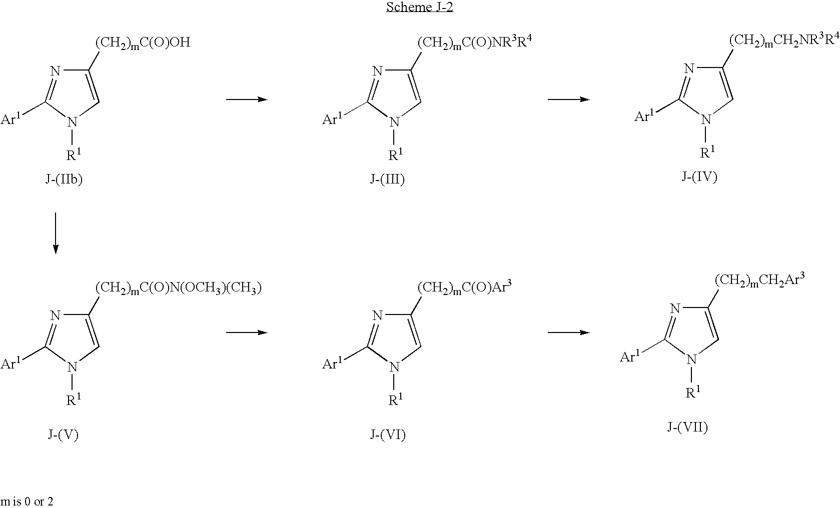

Reaction of the acid J-(IIb) with the appropriately substituted amine under standard coupling procedures provides the desired amide J-(III). Reduction of the amide with common reducing agents such as diborane or lithium aluminum hydride provides the corresponding amine J-(IV). Alternatively treatment of the acid J-(IIb) with Weinreb's reagent provides amide J-(V). Treatment of the amide under standard condition with an organometallic reagent (ex. aryl lithium or aryl magnesium halide) provides the ketone J-(VI). Reduction of the ketone under a variety of conditions affords the desired product J-(VII).

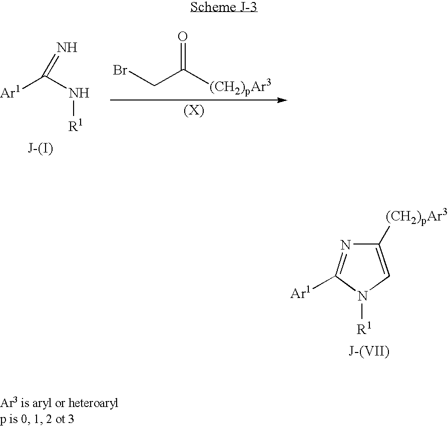

Alternatively treatment of amidine J-(I) with J-(X) provides the desired imidazole J-(VII).

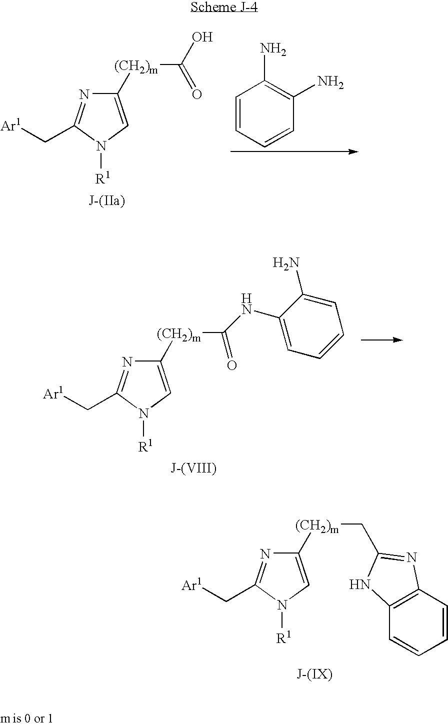

An alternative route to obtain heteroaryl derivatives is to react the activated acid of J-(IIb) with the appropriate substrate followed by cyclization to provide the desired product. For example as depicted in Scheme J-4, reaction of the activated acid of J-(IIb) with benzene-1,2-diamine provides the intermediate amide J-(VIII), which is cyclized to afford the benzimidazole derivative J-(IX).

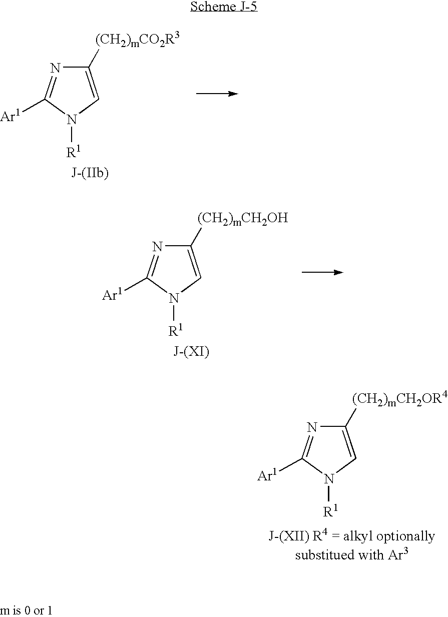

Treatment of carboxylic acid J-(IIb) under standard reducing conditions (e.g., lithium aluminum hydride) gives J-(XI). Treatment of J-(XI) under standard ether forming conditions (e.g., NaH, halo-R⁴) gives J-(XII).

Compounds are prepared in a manner essentially as described above and in the general schemes.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214; Sinkula, A. A.; Yalkowsky. Journal of Pharmaceutical Sciences 1975, 64, 181-210; Verbiscar, A. J.; Abood, L. G Journal of Medicinal Chemistry 1970, 13, 1176-1179; Stella, V. J.; Himmelstein, K. J. Journal of Medicinal Chemistry 1980, 23, 1275-1282; Bodor, N.; Kaminski, J. J. Annual Reports in Medicinal Chemistry 1987, 22, 303-313.

The compounds of this invention may be modified by appending appropriate functionalities to enhance selective biological properties. Such modifications are known in the art and include those which increase biological penetration into a given biological compartment (e.g., blood, lymphatic system, nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺salts. This invention also envisions the quaternization of any basic nitrogen-containing groups of the compounds disclosed herein. Water or oil-soluble or dispersible products may be obtained by such quaternization.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including ion channel-mediated disorders or symptoms thereof. References which include examples of additional therapeutic agents are: 1) Burger's Medicinal Chemistry & Drug Discovery 6^{th} edition, by Alfred Burger, Donald J. Abraham, ed., Volumes 1 to 6, Wiley Interscience Publication, NY, 2003; 2) Ion Channels and Disease by Francis M. Ashcroft, Academic Press, NY, 2000; and 3) Calcium Antagonists in Clinical Medicine 3^{rd} edition, Murray Epstein, MD, FACP, ed., Hanley & Belfus, Inc., Philadelphia, Pa., 2002. Additional therapeutic agents include but are not limited to agents for the treatment of cardiovascular disease (e.g., hypertension, angina, atrial fibrillation, prevention of stroke, heart failure, acute myocardial ischemia, etc), metabolic disease (e.g., syndrome X, diabetes, obesity), renal or genito-urinary disease (e.g, glomerular nephritis, urinary incontinence, nephrotic syndrome), and their disease symptoms. Examples of additional therapeutic agents for treatment of cardiovascular disease and disease symptoms include but are not limited to antihypertensive agents, ACE inhibitors, angiotensin II receptor antagonists, statins, β-blockers, antioxidants, anti-inflammatory drugs, anti-thrombotics, anti-coagulants or antiarrythmics. Examples of additional therapeutic agents for treatment of metabolic disease and disease symptoms include but are not limited to ACE inhibitors, angiotensin II antagonists, fibrates, thiazolidinediones or sulphonylurea anti-diabetic drugs. Examples of additional therapeutic agents for treatment of renal and/or genitor-urinary syndromes and their symptoms include but are not limited to alpha-I adrenergic antagonists (e.g., doxazosin), anti-muscarinics (e.g., tolterodine), norepinephrine/serotonin reuptake inhibitors (e.g., duloxetine), tricyclic antidepressants (e.g., doxepin, desipramine) or steroids.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-δ-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The invention will be further described in the following examples. It should be understood that these examples are for illustrative purposes only and are not to be construed as limiting this invention in any manner.

### EXAMPLE A1

### Oocyte Assay

Representative compounds of the formulae herein are screened for activity against calcium channel targets in an assay essentially as described in Neuron January 1997, 18(11): 153-166, Lin et. al.; J. Neurosci. Jul. 1, 2000,20(13):4768-75, J. Pan and D. Lipsombe; and J. Neurosci., Aug. 15, 2001, 21(16):5944-5951, W. Xu and D. Lipscombe, using Xenopus oocyte heterologeous expression system. The assay is performed on various calcium channels (e.g., Caᵥ1.2 or Caᵥ1.3 subfamily) whereby the modulation of the calcium channel is measured for each compound.

### EXAMPLE A2

### HEK Assay

HEK-293T/17 cells are transiently transfected in a similar manner as described in FuGENE 6 Package Insert Version 7, April 2002, Roche Applied Science, Indianapolis, Ind. The cells are plated at 2.5×10⁵ cells in 2 mL in a 6-well plate in incubator for one night and achieve a 30-40% confluence. In a small sterile tube, add sufficient serum-free medium as diluent for FuGENE Transfection Reagent (Roche Applied Science, Indianapolis, Ind.), to a total volume of 100 μL. Add 3 μL of FuGENE 6 Reagent directly into this medium. The mixture is tapped gently to mix. 2 μg of DNA solution (0.8-2.0 μg/μL) is added to the prediluted FuGENE 6 Reagent from above. The DNA/Fugene 6 mixture is gently pipeted to mix the contents and incubated for about 15 minutes at room temperature. The complex mixture is then added to the HEK-293T/17 cells, distributing it around the well, and swirled to ensure even dispersal. The cells are returned to the incubator for 24 hrs. The transfected cells are then replated at density 2.5×10⁵ in a 35 mm dish with 5 glass coverslips and grow in low serum(1%) media for 24 hrs. Coverslips with isolated cells are then transferred into chamber and calcium channel (e.g., L-type, N-type, etc.) current or other currents for counter screening are recorded from the transiently transfected HEK-293T/17 cells.

The whole-cell voltage clamp configuration of the patch clamp technique is employed to evaluate voltage-dependent calcium currents essentially as described by Thompson and Wong (1991) J. Physiol., 439: 671-689. To record calcium channel (e.g., L-type, N-type, etc.) currents for evaluation of inhibitory potency of compounds (steady-state concentration-response analysis), five pulses of 20-30 ms voltage steps to about +10 mV (the peak of the current voltage relationship) are delivered at five Hz every 30 second from a holding potential at −100 mV. Compound evaluations were carried out essentially as described by Sah D W and Bean B P (1994) Mol Pharmacol. 45(1): 84-92.

### EXAMPLE A3

### Formalin Test

Representative compounds of the formulae herein are screened for activity in the formalin test. The formalin test is widely used as a model of acute and tonic inflammatory pain (Dubuisson & Dennis, 1977 Pain 4:161-174; Wheeler-Aceto et al, 1990, Pain 40:229-238; Coderre et al, 1993, Pain 52:259-285). The test involves the administration to the rat hind paw of a dilute formalin solution followed by monitoring behavioral signs (i.e., flinching, biting and licking) during the “late phase” (11 to 60 minutes post injection) of the formalin response which reflects both peripheral nerve activity and central sensitization. Male, Sprague-Dawley rats (Harlan, Indianapolis, Ind.) weighing approximately 225-300g are used with an n=6−8 for each treatment group.

Depending on pharmacokinetic profile and route of administration, vehicle or a dose of test compound is administered to each rat by the intraperitoneal or oral route 30-120 minutes prior to formalin. Each animal is acclimated to an experimental chamber for 60 minutes prior to formalin administration, which is 50 μL of a 5% solution injected subcutaneously into the plantar surface of one hind paw using a 300 μL microsyringe and a 29 gauge needle. A mirror is angled behind the chambers to enhance the views of the animals' paws. The number of flinches (paw lifts with or without rapid paw shaking) and the time spent biting and/or licking the injured hind paw are recorded for each rat for 2 continuous minutes every 5 minutes for a total of 60 minutes after formalin administration. A terminal blood sample is harvested for analysis of plasma compound concentrations. Between groups comparisons of the total number of flinches or time spent biting and/or licking during the early or late phase are conducted using one-way analysis of variance (ANOVA). P<0.05 was considered statistically significant and p=0.05−1.0 was considered evidence of a statistical trend. Data were presented graphically as mean ±S.E.M. for each 5-minute interval of the 60-minute experimental observation period. Compounds were considered efficacious based on their ability to inhibit the number of flinches or the time spent biting and/or licking during the late phase of the formalin response. Representative compounds of the formulae herein are evaluated for activity against calcium channel targets.

Representative compounds of the formulae herein were evaluated for activity against calcium channel targets.

### EXAMPLE A4

### Method A

### Compound A1 (compound 1 of Scheme A5)

### {2-[2-(1H-Benzoimidazol-2-yl)-ethylsulfanyl]-3-p-tolyl-3H-imidazol-4-ylmethyl}-(4-fluoro-phenyl)-amine

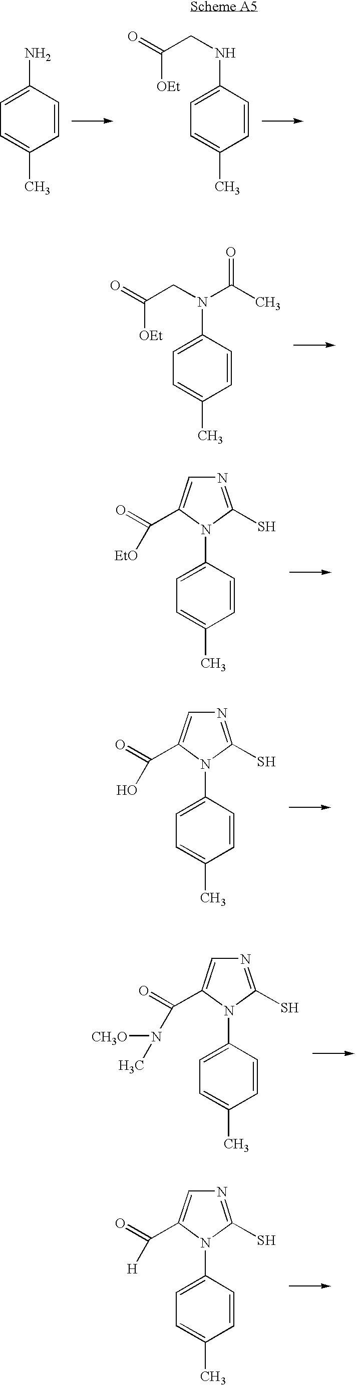

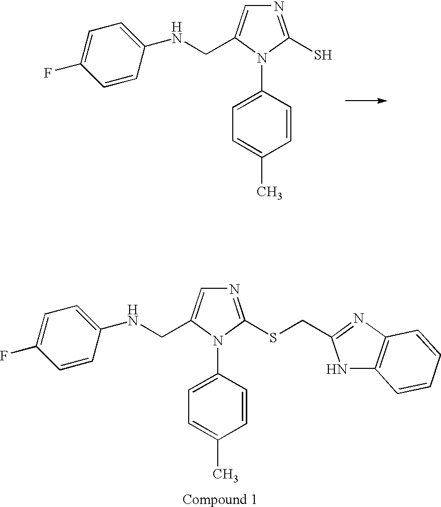

### Part 1. Preparation of p-Tolylamino-acetic acid ethyl ester

A mixture of p-toluidine (16.6 g, 155 mmol), sodium acetate (16.5 g, 201.5 mmol) in ethanol (200 mL) was stirred and ethyl bromoacetate (16.5 mL, 155 mmol) was added at room temperature. The mixture was heated at 80° C. for 1 hour then cooled to room temperature. The mixture was quenched with water and extracted with ethyl acetate. The organics were dried and concentrated under vacuum. The resulting residue was purified by chromatography (SiO₂, 20% ethyl acetate in n-hexane to give p-tolylamino-acetic acid ethyl ester (23.9 g, 124 mmol) as a white solid.

### Part 2. Preparation of (Acetyl-p-tolyl-amino)-acetic acid ethyl ester

A cooled solution of p-tolylamino-acetic acid ethyl ester (23.9 g, 124 mmol) in THF (300 mL) was stirred and acetyl chloride (10.5 mL, 148 mmol) was slowly added. The mixture was stirred for 1 hour and quenched with water and extracted with ethyl acetate. The organics were dried and concentrated under vacuum to give (acetyl-p-tolyl-amino)-acetic acid ethyl ester (14.3 g, 96 mmol) as a white solid.

### Part 3. Preparation of 2-Mercapto-3-p-tolyl-3H-imidazole-4-carboxylic acid ethyl ester

A solution of p-tolylamino-acetic acid ethyl ester (5.0 g, 21.3) and ethyl formate (5.3 g, 71.3 mmol) in benzene (10 mL) was cooled to 0° C. and potassium ethoxide (21.3 mmol) was added. The mixture was placed in a refrigerator to stand overnight and was extracted with water. To the aqueous solution was added potassium thiocyanate (2.14 g, 22.0 mmol) and concentrated aqueous HCl (4 mL). The mixture was heated for 2 hours at 60° C. then cooled. The mixture was extracted with ethyl acetate. The organics were dried and concentrated under vacuum to give 2-mercapto-3-p-tolyl-3H-imidazole-4-carboxylic acid ethyl ester (1.7 g, 6.5 mmol) as a white solid.

### Part 4. Preparation of 2-Mercapto-3-p-tolyl-3H-imidazole-4-carboxylic acid

A solution of 2-mercapto-3-p-tolyl-3H-imidazole-4-carboxylic acid ethyl ester (1.2 g, 4.6 mmol) in 1,4-dioxane (10 mL) was stirred and lithium hydroxide hydrate (1 M, 10 mL) was added and the mixture was stirred at room temperature for 2 hours. The mixture was neutralized with aqueous 2N HCl and extracted with ethyl acetate. The organics were dried and concentrated under vacuum to give 2-mercapto-3-p-tolyl-3H-imidazole-4-carboxylic acid (1g, 4,3 mmol) as a white solid.

### Part 5. Preparation of 2-Mercapto-3-p-tolyl-3H-imidazole-4-carboxylic acid methoxy-methyl-amide

A mixture of 2-mercapto-3-p-tolyl-3H-imidazole-4-carboxylic acid (0.468 g, 2 mmol), 1-ethyl-3-(3-dimethylaminopropyl)-carbodiimide hydrochloride (0.382 g, 2 mmol) and N,O-dimethylhydroxylamine (0.195 g, 2 mmol) in pyridine (4 mL) was heated at 40° C. overnight. The mixture was cooled, quenched with water and extracted with ethyl acetate. The organics were dried, concentrated under vacuum to give 2-mercapto-3-p-tolyl-3H-imidazole-4-carboxylic acid methoxy-methyl-amide (0.245 g, 0.88 mmol) as an oil.

### Part 6. Preparation of 2-Mercapto-3-p-tolyl-3H-imidazole-4-carbaldehyde

A mixture of lithium aluminum hydride (0.10 g, 2.65 mmol) in tetrahydrofuran (10 mL) was stirred under nitrogen blanket at 0° C. and 2-mercapto-3-p-tolyl-3H-imidazole-4-carboxylic acid methoxy-methyl-amide (0.245 g, 0.88 mmol) in THF (5 mL) was added. The mixture was allowed to warm to room temperature and stir for 2 hours. The mixture was cooled to 0° C. and quenched with aqueous 15% sodium bicarbonate and water then extracted with ethyl acetate. The organics were dried and concentrated under vacuum to give 2-mercapto-3-p-tolyl-3H-imidazole-4-carbaldehyde (0.176 g, 0.80 mmol) as a solid.

### Part 7. Preparation of 2-Mercapto-3-p-tolyl-3H-imidazole-4-carboxylic acid (4-fluoro-phenyl)-amide

A solution of 2-mercapto-3-p-tolyl-3H-imidazole-4-carbaldehyde (0.176 g, 0.80 mmol) and 4-fluoroaniline (0.80 mmol, 88 mgs) in DMF/acetic acid (10/1:v/v, 3 mL) was stirred at room temperature for 1 hour. Sodium cyanoborohydride (0.76 g, 1.2 mmol) was added and the mixture stirred overnight. The mixture was quenched with water and extracted with ethyl acetate. The organics were dried and concentrated to give a residue. Purification by chromatography (SiO₂, 5% methanol in methylene chloride) gave 2-mercapto-3-p-tolyl-3H-imidazole-4-carboxylic acid (4-fluoro-phenyl)-amide (0.05 g, 0.16 mmol) as a solid.

### Part 8. Preparation of 2-[2-(1H-Benzoimidazol-2-yl)-ethylsulfanyl]-3-p-tolyl-3H-imidazol-4-ylmethyl}-(4-fluoro-phenyl)-amine

A mixture of 2-mercapto-3-p-tolyl-3H-imidazole-4-carboxylic acid (4-fluoro-phenyl)-amide (0.05 g, 0.16 mmol)) and 2-(chloromethyl)benzimidazole (0.032 g, 0.19 mmol) in acetone (5 mL) was stirred and potassium carbonate (0.048 g, 0.35 mmol) was added. The mixture was heated at 40° C. for 2 hours and cooled. The mixture was quenched with water and extracted with ethyl acetate. The organics were dried and concentrated under vacuum to give a solid. The solid was dissolved in diethyl ether and a solution of etheral-HCl was added. The mixture was concentrated under vacuum to give 2-[2-(1H-Benzoimidazol-2-yl)-ethylsulfanyl]-3-p-tolyl-3H-imidazol-4-ylmethyl}-(4-fluoro-phenyl)-amine (0.042 g, 0.08 mmol) as the HCl salt.

### Method B

### Compound 2 (Compound 2 of Scheme A)

### 2-(1H-Benzoimidazol-2-ylmethylsulfanyl)-3-p-tolyl-3H-imidazole-4-carboxylic acid (4-fluoro-phenyl)-amide

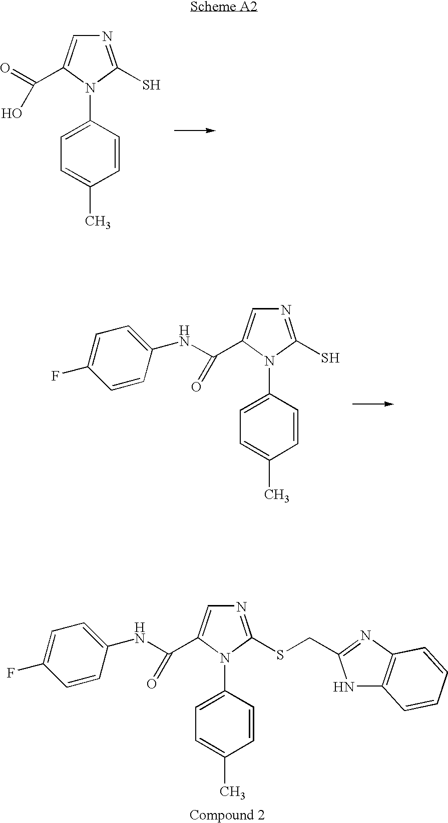

### Part 1. Preparation of 2-Mercapto-3-p-tolyl-3H-imidazole-4-carboxylic acid (4-fluoro-phenyl)-amide

A mixture of 2-mercapto-3-p-tolyl-3H-imidazole-4-carboxylic acid (0.468 g, 2 mmol), 1-ethyl-3-(3-dimethylaminopropyl)-carbodiimide hydrochloride (0.382 g, 2 mmol) and 4-fluoroaniline (0.222 g, 2 mmol) in pyridine (4 mL) was heated at 40° C. overnight. The mixture was cooled and quenched with water and extracted with ethyl acetate. The organics were dried, concentrated under vacuum to give 2-mercapto-3-p-tolyl-3H-imidazole-4-carboxylic acid (4-fluoro-phenyl)-amide (0.206 g, 0.63 mmol) as an oil.

### Part 2. Preparation of 2-(1H-Benzoimidazol-2-ylmethylsulfanyl)-3-p-tolyl-3H-imidazole-4-carboxylic acid (4-fluoro-phenyl)-amide

To a solution of 2-mercapto-3-p-tolyl-3H-imidazole-4-carboxylic acid (4-fluoro-phenyl)-amide (0.206 g, 0.63 mmol) and 2-(chloromethyl)benzimidazole (0.166 g, 1.00 mmol) in acetone (10 mL) was stirred and potassium carbonate (0.191 g, 1.12 mmol) was added. The mixture was heated at 40° C. for 2 hours and cooled. The mixture was quenched with water and extracted with ethyl acetate. The organics were dried and concentrated under vacuum to give a solid. Purification by chromatography (SiO₂, 20% acetone in n-hexane) gave 2-(1H-benzoimidazol-2-ylmethylsulfanyl)-3-p-tolyl-3H-imidazole-4-carboxylic acid (4-fluoro-phenyl)-amide (0.133 g, 0.29 mmol) as a white solid.

### EXAMPLE B4

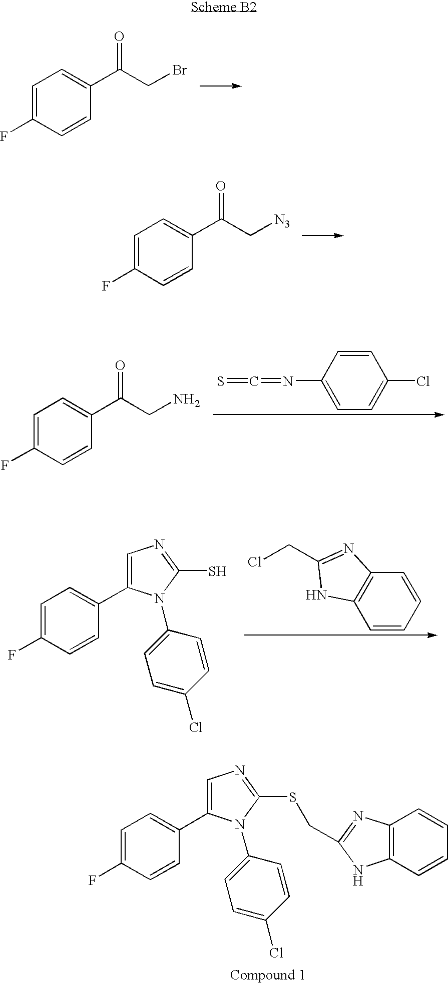

### Compound 1 (Compound 1 of Scheme B2

### 2-[1-(4-Chloro-phenyl)-5-(4-fluoro-phenyl)-1H-imidazol-2-ylsulfanylmethyl]-1H-benzoimidazole

### Part 1. Preparation of 2-Azido-1-(4-fluoro-phenyl)-ethanone

A solution of 2-bromo-1-(4-fluoro-phenyl)-ethanone (2.5 g, 11.5 mmol) in DMSO (15 mL) at 10° C. was vigorously stirred and sodium azide (0.94 g, 14.4 mmol) was added. The mixture was stirred for 1 hour then quenched with water (20 mL) and extracted with ethyl acetate (2×25 mL). The combined organic layers were washed with water (25 mL) and brine (25 mL), dried over anhydrous sodium sulfate, filtered and concentrated under reduce pressure to give 2-azido-1-(4-fluoro-phenyl)-ethanone (1.7 g, 9.3 mmol) as a viscous yellow-red liquid.

### Part 2. Preparation of 2-Amino-1-(4-fluoro-phenyl)-ethanone hydrochloride

To a solution of 2-azido-1-(4-fluoro-phenyl)-ethanone (8.0 g, 44.7 mmol) in ethanol (125 mL) was added concentrated aqueous HCl (6 mL) and 10% Pd/C (10 mol %). The mixture was stirred under hydrogen (H₂) atmosphere at 45 psi for 1 hour. The mixture was filtered through celite and the celite cake was washed with copious amounts of methanol. The solvent was under reduce pressure and the semisolid was triturated with diethyl ether, filtered and dried to give 2-amino-1-(4-fluoro-phenyl)-ethanone hydrochloride (5.0 g, 26.5 mmol) as a white crystalline solid.

### Part 3. Preparation of 1-(4-Chloro-phenyl)-5-(4-fluoro-phenyl)-1H-imidazole-2-thiol

A mixture of 2-amino-1-(4-fluoro-phenyl)-ethanone hydrochloride (5.0 g, 26.5 mmol), 4-chlorophenyl isothiocyanate (4.49 g, 26.5 mmol) and sodium hydrogencarbonate (3.3 g, 39.7 mmol) in ethanol (100 mL) was heated at 90° C. for 2 hours. The solvent was removed under reduce pressure. The resulting residue was re-suspended in aqueous 1N sodium hydroxide (50 mL) and heated at 100° C. overnight. The hot mixture was filtered, cooled and carefully acidified with aqueous 6N HCl. The resulting mixture was filtered to give 1-(4-chloro-phenyl)-5-(4-fluoro-phenyl)-1H-imidazole-2-thiol (8.0 g, 26.3 mmol) as a yellow solid after drying.

### Part 4. Preparation of 2-[1-(4-Chloro-phenyl)-5-(4-fluoro-phenyl)-1H-imidazol-2-yl-sulfanylmethyl]-1H-benzoimidazole

A mixture of 1-(4-chloro-phenyl)-5-(4-fluoro-phenyl)-1H-imidazole-2-thiol (4.0 g, 13.2 mmol), 2-(chloromethyl)benzimidazole (2.2 g, 13.2 mmol) and potassium carbonate (5.5 g, 39.6 mmol) in acetone (50 mL) was heated at 75° C. until all starting materials were consumed. The mixture was cooled and the solvent was removed under reduce pressure. The resulting residue was partitioned in 1:1:1 water/ethyl acetate/hexane. The brown solid was filtered, dried and re-suspended in minimal amount of methanol. The methanolic mixture was filtered and dried to obtain a white solid. The solid was re-suspended in methanol and treated with ethereal 2N HCl until a solution persisted. The solution was diluted with a large amount of diethyl ether to promote precipitation, filtered and dried to give 2-[1-(4-chloro-phenyl)-5-(4-fluoro-phenyl)-1H-imidazol-2-yl-sulfanylmethyl]-1H-benzoimidazole (3.5 g, 7.4 mmol) as a white solid.

### EXAMPLE C4

### Method CA

### Compound C1 (Compound 1 in Scheme C4)

### {5-[(4-Fluoro-phenylamino)-methyl]-4-p-tolyl-4H-[1,2,4]triazol-3-ylsulfanyl}-acetic acid

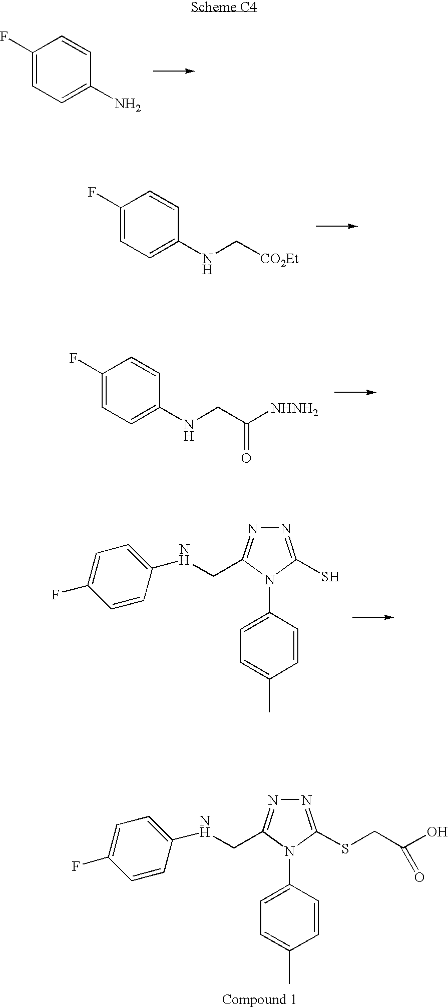

### Part 1. Preparation of (4-Fluoro-phenylamino)-acetic acid ethyl ester

A mixture of 4-fluoroaniline (10 g, 90 mmol), bromoacetate (15 g, 90 mmol), and sodium acetate (11 g, 135 mmol) in ethanol (200 mL) was heated reflux for 2 hours. The cooled reaction was cooled and concentrated under vacuum. The residue was diluted with water and extracted with ethyl acetate. The organics were dried and concentrated under vacuum to give (4-fluoro-phenylamino)-acetic acid ethyl ester (8.46 g, 42.9 mmol) as a white solid.

### Part 2. Preparation of (4-Fluoro-phenylamino)-acetic acid hydrazide

A mixture of (4-fluoro-phenylamino)-acetic acid ethyl ester and hydrazine (8.23 g, 257 mmol) in ethanol (200 mL) was refluxed for 3 hours. The reaction was cooled and concentrated under vacuum. Trituration of the residue with n-hexane (75 mL) gave (4-fluoro-phenylamino)-acetic acid hydrazide (5.5 g, 30.20 mmol) as a white solid.

### Part 3. Preparation of 5-[(4-Fluoro-phenylamino)-methyl]-4-p-tolyl-2,4-dihydro-[1,2,4]triazole-3-thione

A mixture of (4-fluoro-phenylamino)-acetic acid hydrazide and p-Tolueneisotliocyanate (4.5 g, 30.20 mmol) in aqueous 2N sodium hydroxide (100 mL) and heated for several hours then cooled. The solution was neutralized with 6N hydrochloric acid and extracted with ethyl acetate. The organics were dried and concentrated under vacuum to give a brown residue. Trituration of the residue with n-hexane (100 mL) gave 5-[(4-fluoro-phenylamino)-methyl]-4-p-tolyl-2,4-dihydro-[1,2,4]triazole-3-thione (7.6 g, 24.2 mmol) as a white solid.

### Part 4. Preparation of {5-[(4-Fluoro-phenylamino)-methyl]-4-p-tolyl-4H-[1,2,4]triazol-3-ylsulfanyl}-acetic acid

To a mixture of 5-[(4-fluoro-phenylamino)-methyl]-4-p-tolyl-2,4-dihydro-[1,2,4]triazole-3-thione (0.30 gm, 0.95 mmol), bromoacetic acid (0.13 gm, 0.95 mmol) and potassium carbonate (0.16 gm, 1.14 mmol) in acetone (6 mL) was heated at 40 C for 3 hours then cooled. The reaction was diluted with water and extracted with ethyl acetate. The organics were dried and concentrated under vacuum to give a residue. The residue was purified by chromatography on silica (20% methanol in methylene chloride) to give {5-[(4-Fluoro-phenylamino)-methyl]-4-p-tolyl-4H-[1,2,4]triazol-3-ylsulfanyl}-acetic acid (0.27 gm, 0.72 mmol) as a white solid.

### Method CB

### Compound C2 (Compound 2 in Scheme C5)

### N-(2-Chloro-phenyl)-2-{5-[(4-fluoro-phenylamino)-methyl]-4-p-tolyl-4H-[1,2,4]triazol-3-ylsulfanyl}-acetamide

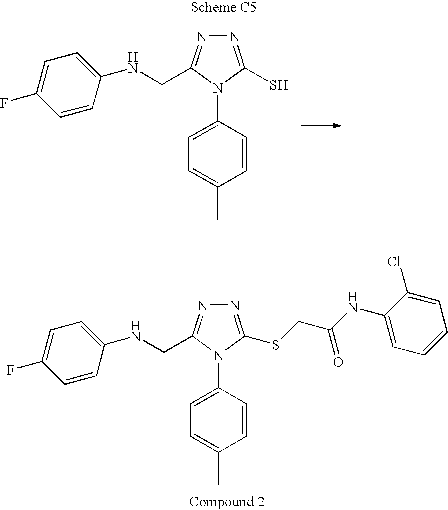

### Part 1. Preparation of N-(2-Chloro-phenyl)-2-{5-[(4-fluoro-phenylamino)-methyl]-4-p-tolyl-4H-[1,2,4]triazol-3-ylsulfanyl}-acetamide

To a mixture of 5-[(4-fluoro-phenylamino)-methyl]-4-p-tolyl-2,4-dihydro-[1,2,4]triazole-3-thione (0.30 gm, 0.95 mmol), 2-bromo-N-(2-chloro-phenyl)-acetamide (0.24 gm, 0.95 mmol) and potassium carbonate (0.16 gm, 1.14 mmol) in acetone (6 mL) was heated at 40 C for 3 hours then cooled. The reaction was diluted with water and extracted with ethyl acetate. The organics were dried and concentrated under vacuum to give a residue. The residue was purified by chromatography on silica (20% methanol in methylene chloride) to give N-(2-Chloro-phenyl)-2-{5-[(4-fluoro-phenylamino)-methyl]-4-p-tolyl-4H-[1,2,4]triazol-3-ylsulfanyl}-acetamide (0.10 gm, 0.20 mmol) as a white solid.

### Method CC

### Compound C3 (Compound 3 in Scheme C6)

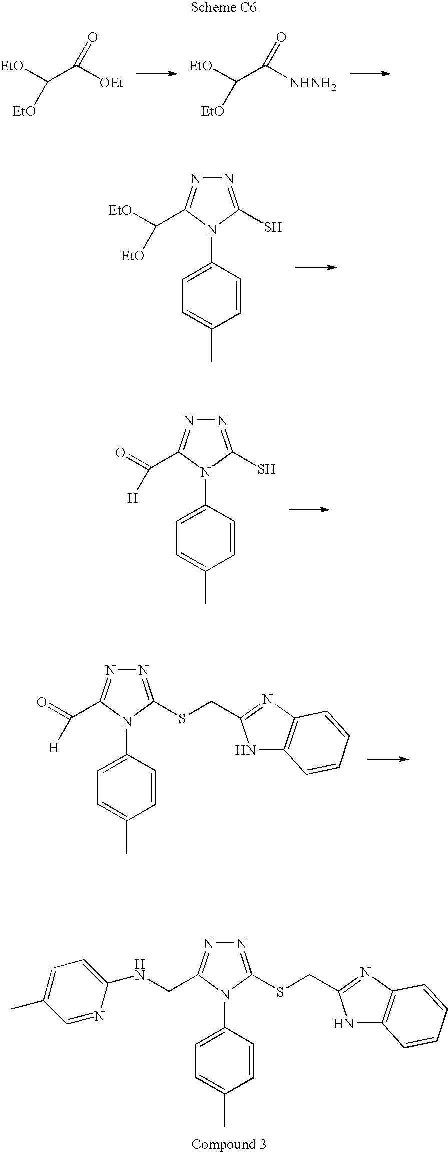

### [5-(1H-Benzoimidazol-2-ylmethylsulfanyl)-4-p-tolyl-4H-[1,2,4]triazol-3-ylmethyl]-(5-methyl-pyridin-2-yl)-amine

### Part 1. Preparation of Diethoxy-acetic acid hydrazide

A solution of ethyl diethoxyacetate (15.8 g, 90 mmol) ethanol (100 mL) was stirred and hydrazine (8.23 g, 257 mmol) was added. The mixture was heated at reflux for 2 hours. then cooled and concentrated under vacuum. The residue was diluted with water and extracted with ethyl acetate. The organics were dried and concentrated under vacuum to give diethoxy-acetic acid hydrazide (10.53 g, 65 mmol) as a clear oil.

### Part 2. Preparation of 5-Diethoxymethyl-4-p-tolyl-2,4-dihydro-[1,2,4]triazole-3-thione

p-Tolueneisothiocyanate (9.7 g, 65 mmol) and diethoxy-acetic acid hydrazide (10.53 g, 65 mmol) was dissolved in aqueous 2N sodium hydroxide (100 mL) and heated for several hours then cooled. The solution was neutralized with 6N hydrochloric acid and extracted with ethyl acetate. The organics were dried and concentrated under vacuum to give a yellow residue. Trituration of the residue with n-hexane (100 mL) gave 5-diethoxymethyl-4-p-tolyl-2,4-dihydro-[1,2,4]triazole-3-thione (12.3 g, 42 mmol) as a yellow solid.

### Part 3. Preparation of 5-Thioxo-4-p-tolyl-4,5-dihydro-1H-[1,2,4]triazole-3-carbaldehyde

A solution of 5-diethoxymethyl-4-p-tolyl-2,4-dihydro-[1,2,4]triazole-3-thione (5 g, 17 mmol) and Aqueous 3N HCl (30 mL) in 1,4-dioxane (10 mL) was stirred and heated at 40 C for 2 hours then cooled. The mixture was quenched with water and extracted with ethyl acetate. The organics dried and concentrated to give a residue. Purification by flash chromatography (SiO₂, 10% acetone in n-hexane) gave 5-thioxo-4-p-tolyl-4,5-dihydro-1H-[1,2,4]triazole-3-carbaldehyde (2.6 g, 11.8 mmol) as a yellow solid.

### Part 4. Preparation of 5-(1H-Benzoimidazol-2-ylmethylsulfanyl)-4-p-tolyl-4H-[1,2,4]triazole-3-carbaldehyde

A solution of 5-thioxo-4-p-tolyl-4,5-dihydro-1H-[1,2,4]triazole-3-carbaldehyde (2.6 g, 1.8 mmol) and 2-(chloromethyl)benzimidazole (2.15 g, 12.9 mmol) in acetone (10 mL) was stirred and potassium carbonate (2.07 g, 15 mmol) was added. The mixture was heated at 40 C for 3 hours then cooled. The mixture was quenched with water and extracted with ethyl acetate. The organics dried and concentrated to give a residue. Purification by flash chromatography (SiO₂, 30% acetone in n-hexane) gave 5-(1H-benzoimidazol-2-ylmethylsulfanyl)-4-p-tolyl-4H-[1,2,4]triazole-3-carbaldehyde (2.50 g, 7.08 mmol) as white solid.

### Part 5. Preparation of [5-(1H-Benzoimidazol-2-ylmethylsulfanyl)-4-p-tolyl-4H-[1,2,4]triazol-3-ylmethyl]-(5-methyl-pyridin-2-yl)-amine

A solution of 5-(1H-benzoimidazol-2-ylmethylsulfanyl)-4-p-tolyl-4H-[1,2,4]triazole-3-carbaldehyde (1.0 g, 4.56 mmol) and 2-amino-5-methyl pyridine (0.49 g, 4.56 mmol) in DMF/HOAc (10/1:v/v) (10 mL) was stirred at room temperature for 1 hour. Sodium cyanoborohydride (0.376 g, 6.0 mmol) was added and mixture was stirred overnight. The mixture was quenched with water and extracted with ethyl acetate. The organics dried and concentrated to give a residue. Purification by flash chromatography (SiO₂, 30% acetone in n-hexane) gave [5-(1H-Benzoimidazol-2-ylmethylsulfanyl)-4-p-tolyl-4H-[1,2,4]triazol-3-ylmethyl]-(5-methyl-pyridin-2-yl)-amine (0.108 g, 0.25 mmol) as an off-white solid.

### EXAMPLE D-4

### Compound D-15

### 3-(2-(1H-Benzo[d]imidazol-2-yl)ethyl)-5-(4-fluorophenyl)-1-p-tolyl-1H-imidazole-2(3H)-thione

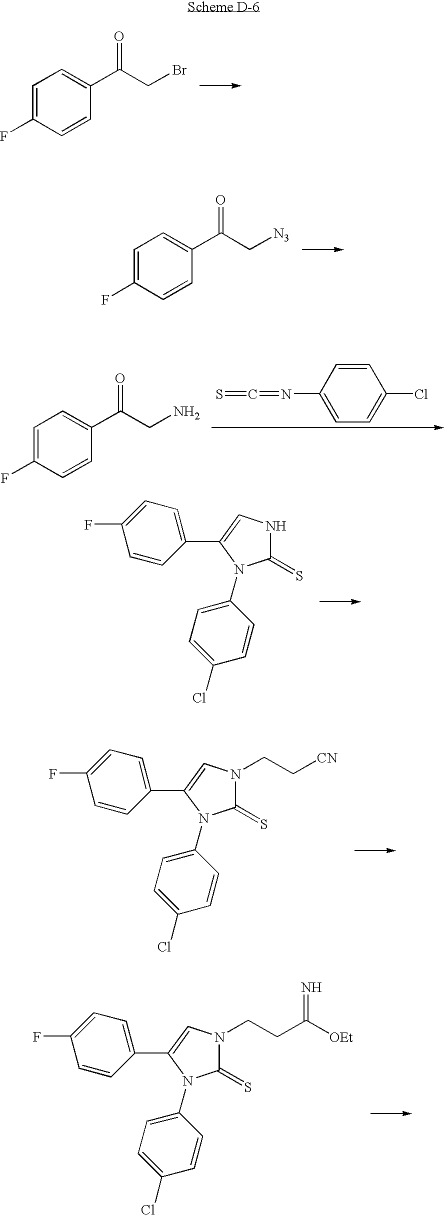

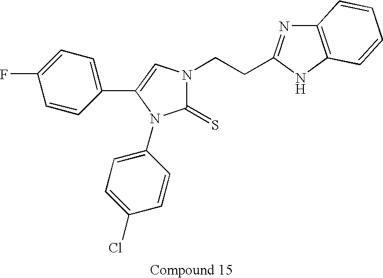

### Part 1. Preparation of 2-Azido-1-(4-fluoro-phenyl)-ethanone

A solution of 2-bromo-1-(4-fluoro-phenyl)-ethanone (1 eq) in DMSO at 10° C. is vigorously stirred and sodium azide (1.25 eq) is added. The mixture is stirred for 1 hour then quenched with water and extracted with ethyl acetate (2×). The combined organic layers are washed with water and brine, dried over anhydrous sodium sulfate, filtered and concentrated under reduce pressure to give 2-azido-1-(4-fluoro-phenyl)-ethanone.

### Part 2. Preparation of 2-Amino-1-(4-fluoro-phenyl)-ethanone hydrochloride

To a solution of 2-azido-1-(4-fluoro-phenyl)-ethanone in ethanol is added concentrated HCl (aq) and 10% Pd/C (10 mol %). The mixture is stirred under hydrogen (H₂) atmosphere at 45 psi for 1 hour. The mixture is filtered through celite and the celite cake is washed with copious amounts of methanol. The solvent is removed under reduce pressure and the resulting residue is triturated with diethyl ether, filtered and dried to give 2-amino-1-(4-fluoro-phenyl)-ethanone hydrochloride.

### Part 3. Preparation of 1-(4-Chloro-phenyl)-5-(4-fluoro-phenyl)-1H-imidazole-2-thiol

A mixture of 2-amino-1-(4-fluoro-phenyl)-ethanone hydrochloride (1 eq), 4-chlorophenyl isothiocyanate (1 eq) and sodium hydrogencarbonate (1.5 eq) in ethanol is heated at 90° C. for 2 hours. The solvent is removed under reduce pressure. The resulting residue is re-suspended in aqueous 1N sodium hydroxide and heated at 100° C. overnight. The hot mixture is filtered, cooled and carefully acidified with aqueous 6N HCl. The resulting mixture is filtered to give 1-(4-chloro-phenyl)-5-(4-fluoro-phenyl)-1H-imidazole-2-thiol.

### Part 4. Preparation of 3-(5-(4-Fluorophenyl)-1,2-dihydro-2-thioxo-1-p-tolylimidazol-3-yl)propanenitrile

A mixture 5-(4-fluorophenyl)-1-p-tolyl-1H-imidazole-2-thiol (1 eq) in dioxane is stirred and Triton B is added. The mixture is heated to 70° C. and acrylonitrile (1 eq) is added and heated for 3 hours. The cooled mixture is partitioned between aqueous 0.1N HCl and ethyl acetate. The organic layer is washed with water and brine, dried over sodium sulfate, filtered and the solvent removed under reduce pressure. Flash chromatography (SiO₂) gives 3-(5-(4-fluorophenyl)-1,2-dihydro-2-thioxo-1-p-tolylimidazol-3-yl)propanenitrile.

### Part 5. Preparation of 3-(1-(4-Chlorophenyl)-5-(4-fluorophenyl)-1,2-dihydro-2-thioxoimidazol-3-yl)propionimidic acid ethyl ester

A solution of the propionitrile in 1:1 ethanol/diethylether is cooled in a ice water bath and HCl (g) is carefully bubbled in the solution over 10-20 minutes. The reaction mixture is stirred at room temperature for 2-4 hours and the solvent is removed under reduce pressure to obtain 3-(1-(4-chlorophenyl)-5-(4-fluorophenyl)-1,2-dihydro-2-thioxoimidazol-3-yl)propionimidic acid ethyl ester.

### Part 6. Preparation of 3-(2-(1H-Benzo[d]imidazol-2-yl)ethyl)-5-(4-fluorophenyl)-1-p-tolyl-1H-imidazole-2(3H)-thione

A mixture of the propionimidic acid ethyl ester and benzene-1,2-diamine in ethanol is stirred and heated at 60° C. overnight. The solvent is removed under reduce pressure; the residue is partitioned between ethyl acetate and saturated aqueous sodium bicarbonate. The organic layer is dried over sodium sulfate, filtered and the solvent removed under reduce pressure. Purification by flash chromatography (SiO₂) followed by HCl salt formation (methanol and 2M ethereal HCl) gives 3-(2-(1H-benzo[d]imidazol-2-yl)ethyl)-5-(4-fluorophenyl)-1-p-tolyl-1H-imidazole-2(3H)-thione hydrochloride.

### Compound D-22

### 3-(1-(4-Chlorophenyl)-5-(4-fluorophenyl)-1,2-dihydro-2-thioxoimidazol-3-yl)propionic acid ethyl ester

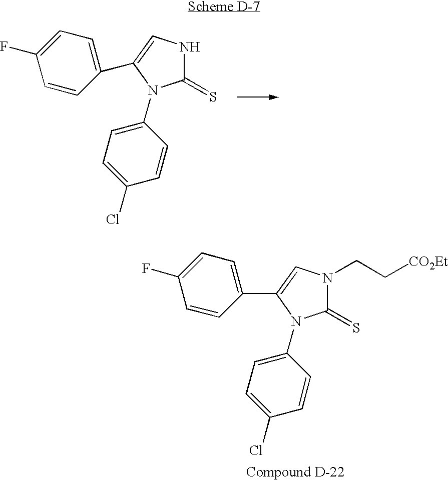

### Preparation of 3-(1-(4-Chlorophenyl)-5-(4-fluorophenyl)-1,2-dihydro-2-thioxoimidazol-3-yl)propionic acid ethyl ester

To a solution 1-(4-chloro-phenyl)-5-(4-fluoro-phenyl)-1H-imidazole-2-thiol (1 eq) in DMF is added a 1M solution of lithium bis(trimethylsilyl)amide in THF (1 eq) and ethyl 3-bromopropionate (1 eq) at room temperature. The mixture is heated at 60° C. for 2 hours and cooled to room temperature. The mixture is quenched with water and extracted with ethyl acetate. The organics were dried and concentrated under vacuum. The residue is purified by chromatography on silica to give 3-(1-(4-chlorophenyl)-5-(4-fluorophenyl)-1,2-dihydro-2-thioxoimidazol-3-yl)propionic acid ethyl ester.

### Compound D-26

### 3-(1-(4-Chlorophenyl)-5-(4-fluorophenyl)-1,2-dihydro-2-thioxoimidazol-3-yl)-1-(pyrrolidin-1-yl)propan-1-one

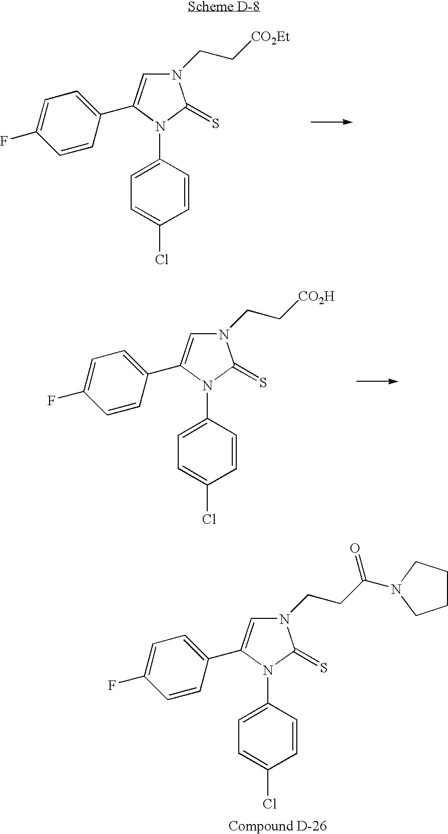

### Part 1. Preparation of 3-(1-(4-Chlorophenyl)-5-(4-fluorophenyl)-1,2-dihydro-2-thioxoimidazol-3-yl)propionic acid

A mixture of 3-(1-(4-chlorophenyl)-5-(4-fluorophenyl)-1,2-dihydro-2-thioxoimidazol-3-yl)propionic acid ethyl ester (1 eq) and lithium hydroxide hydrate (1.2 eq) is dissolved in 1,4-dioxane:water (4/1:v/v) and allowed to stir at room temperature for 3 hours. The reaction mixture is neutralized with aqueous 2N HCl and extracted with ethyl acetate. The organics are dried and concentrated under vacuum to give 3-(1-(4-chlorophenyl)-5-(4-fluorophenyl)-1,2-dihydro-2-thioxoimidazol-3-yl)propionic acid.

### Part 2. Preparation of 3-(1-(4-Chlorophenyl)-5-(4-fluorophenyl)-1,2-dihydro-2-thioxoimidazol-3-yl)-1-(pyrrolidin-1-yl)propan-1-one

To a solution of 3-(1-(4-chlorophenyl)-5-(4-fluorophenyl)-1,2-dihydro-2-thioxoimidazol-3-yl)propionic acid (1 eq), 1-3-(dimethylaminopropyl)-3-ethylcarbodiimide hydrochloride (1.7 eq) and morpholine (1.7 eq) in THF is stirred overnight at room temperature. The reaction is quenched with water and extracted with ethyl acetate. The organics are dried and concentrated under vacuum. The residue is purified by chromatography on silica to give 3-(1-(4-chlorophenyl)-5-(4-fluorophenyl)-1,2-dihydro-2-thioxoimidazol-3-yl)-1-(pyrrolidin-1-yl)propan-1-one.

### EXAMPLE E-4

### Compound E-1

### 3-[3-(2-Methoxy-phenyl)-5-thioxo-4-p-tolyl-4,5-dihydro-[1,2,4]triazol-1-yl]-propionic acid, ethyl ester

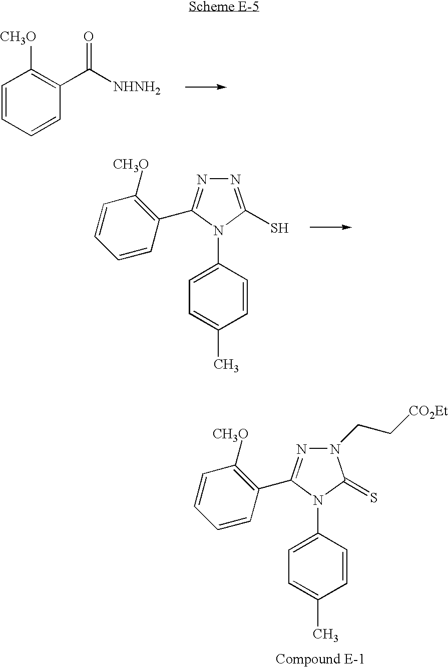

### Part 1. Preparation of 5-(2-Methoxy-phenyl)-4-p-tolyl-4H-[1,2,4]triazole-3-thiol

A mixture of 2-methoxybenzhydrazide (7.0 gm, 42 mmol) and p-Tolyl isothiocyanate (6.3 gm, 42 mmol) in ethanol (100 mL) was heated at reflux for one hour then cooled. The reaction mixture was filtered and the filter cake was washed with cold ethanol (50 mL). The filter cake was dissolved in aqueous 2N sodium hydroxide (100 mL) and heated overnight then cooled. The solution was neutralized with 6N hydrochloric acid and extracted with ethyl acetate. The organics were dried and concentrated under vacuum to give a white solid. Trituration of the solid with ethanol (100 mL) gave 5-(2-Methoxy-phenyl)-4-p-tolyl-4H-[1,2,4]triazole-3-thiol (11 gm, 37 mmol) as a white solid.

### Part 2. Preparation of 3-[3-(2-Methoxy-phenyl)-5-thioxo-4-p-tolyl-4,5-dihydro-[1,2,4]triazol-1-yl]-propionic acid, ethyl ester

To a solution of 5-(2-methoxy-phenyl)-4-p-tolyl-4H-[1,2,4]triazole-3-thiol (0.9 g, 30.3 mmol) in DMF (100 mL) was added a 1M solution of lithium bis(trimethylsilyl)amide in THF (30.3 mL) and ethyl 3-bromopropionate (5.48 g, 30.3 mmol) at room temperature. The mixture was heated at 60° C. for 1 hour and cooled to room temperature. The mixture was quenched with water and extracted with ethyl acetate. The organics were dried and concentrated under vacuum. The residue was purified by chromatography on silica (20% ethyl acetate in n-hexane) to give 3-[3-(2-methoxy-phenyl)-5-thioxo-4-p-tolyl-4,5-dihydro-[1,2,4]triazol-1-yl]-propionic acid, ethyl ester (10.08 g, 25.4 mmol) as a clear oil.

### Compound E-2

### 3-[3-(2-Methoxy-phenyl)-5-thioxo-4-p-tolyl-4,5-dihydro-[1,2,4]triazol-1-yl]-1-morpholin-4-yl-propan-1-one

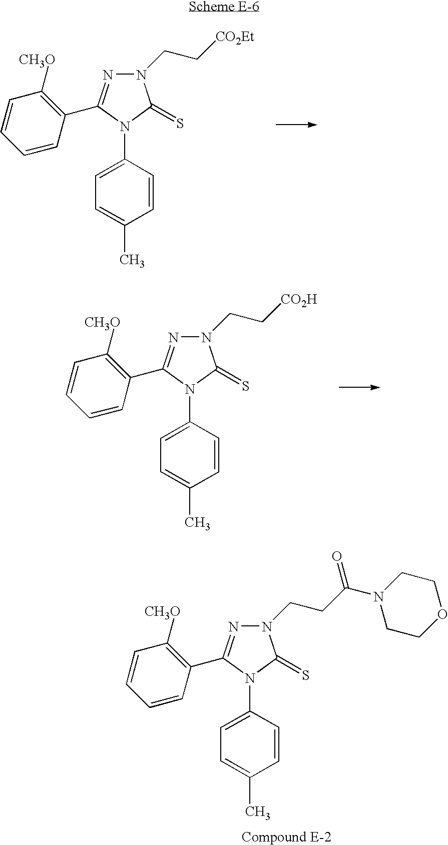

### Part 1. Preparation of 3-[3-(2-Methoxy-phenyl)-5-thioxo-4-p-tolyl-4,5-dihydro-[1,2,4]triazol-1-yl]-propionic acid

A mixture of 3-[3-(2-methoxy-phenyl)-5-thioxo-4-p-tolyl-4,5-dihydro-[1,2,4]triazol-1-yl]-propionic acid, ethyl ester (10.08 g, 25.4 mmol) and lithium hydroxide hydrate (1.28 g, 30.48 mmol) was dissolved in 1,4-dioxane:water (4/1:v/v) and allowed to stir at room temperature for 3 hours. The reaction mixture was neutralized with aqueous 2N HCl and extracted with ethyl acetate. The organics were dried and concentrated under vacuum to give 3-[3-(2-methoxy-phenyl)-5-thioxo-4-p-tolyl-4,5-dihydro-[1,2,4]triazol-1-yl]-propionic acid (8.99 g, 24.4 mmol) as a white solid.

### Part 2. Preparation of 3-[3-(2-Methoxy-phenyl)-5-thioxo-4-p-tolyl-4,5-dihydro-[1,2,4]triazol-1-yl]-1-morpholin-4-yl-propan-1-one

To a solution of 3-[3-(2-methoxy-phenyl)-5-thioxo-4-p-tolyl-4,5-dihydro-[1,2,4]triazol-1-yl]-propionic acid (0.50 g, 1.36 mmol, 1-3-(dimethylaminopropyl)-3-ethylcarbodiimide hydrochloride (0.388 g, 2.03 mmol) and morpholine (0.177 g, 2.03 mmol) in THF (15 mL) was stirred overnight at room temperature. The reaction was quenched with water and extracted with ethyl acetate. The organics were dried and concentrated under vacuum. The residue was purified by chromatography on silica (20% acetone in n-hexane) to give 3-[3-(2-methoxy-phenyl)-5-thioxo-4-p-tolyl-4,5-dihydro-[1,2,4]triazol-1-yl]-1-morpholin-4-yl-propan-1-one (0.286 g, 0.65 mmol) as a white solid.

### Compound E-3

### 2-[2-(1H-Benzoimidazol-2-yl)-ethyl]-5-(2-methoxy-phenyl)-4-p-tolyl-2,4-dihydro-[1,2,4]triazole-3-thione

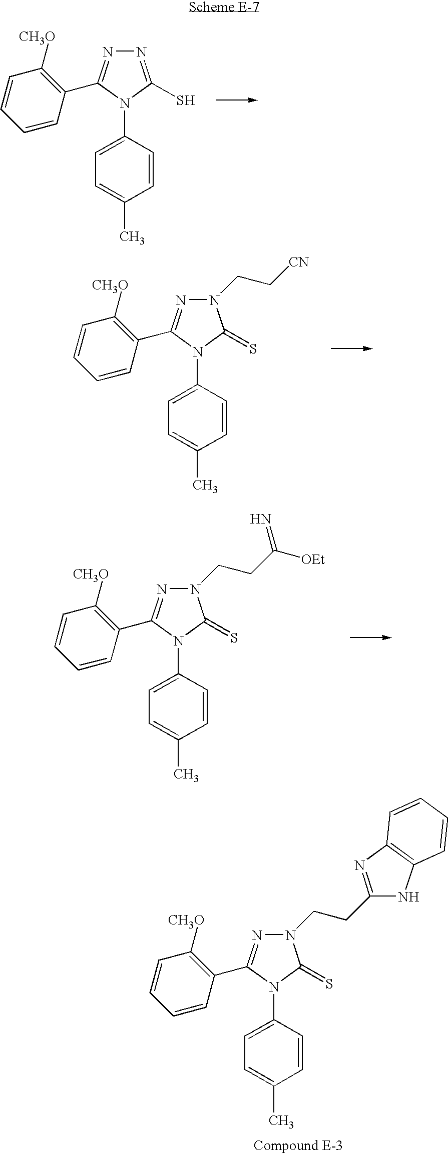

### Part 1. Preparation of 3-[3-(2-methoxy-phenyl)-5-thioxo-4-p-tolyl-4,5-dihydro-[1,2,4]triazol-1-yl]-propionitrile

A mixture of 5-(2-methoxy-phenyl)-4-p-tolyl-2,4-dihydro-[1,2,4]triazole-3-thione (1.1 g, 3.7 mmol) in dioxane (6 mL) was stirred and Triton B (20 drops) was added. The mixture was heated to 70° C. and acrylonitrile (250 μL, 3.7 mmol) was added and heated and additional 3 hours. The cooled mixture was partitioned between aqueous 0.1N HCl (10 mL) and ethyl acetate (20 mL). The organic layer was washed with water (10 mL) and brine (10 mL) and dried over sodium sulfate, filtered and the solvent removed under reduce pressure to give a viscous yellow oil. Flash chromatography (SiO₂, 2:3 ethyl acetate/hexane) gave 3-[3-(2-methoxy-phenyl)-5-thioxo-4-p-tolyl-4,5-dihydro-[1,2,4]triazol-1-yl]-propionitrile (1 g, 2.8 mmol) as a white foam.

### Part 2. Preparation of 3-[3-(2-Methoxy-phenyl)-5-thioxo-4-p-tolyl-4,5-dihydro-[1,2,4]triazol-1-yl]-propionimidic acid ethyl ester

A solution of propionitrile (0.5 g, 1.4 mmol) in 1:1 ethanol/diethylether (20 mL) was cooled in an ice water bath and HCl (g) was carefully bubbled in the solution over 10-20 minutes. The reaction mixture was stirred at room temperature for 2-4 hours and the solvent was removed under reduce pressure to obtain 3-[3-(2-methoxy-phenyl)-5-thioxo-4-p-tolyl-4,5-dihydro-[1,2,4]triazol-1-yl]-propionimidic acid ethyl ester a viscous yellow oil. The oil used immediately without purification.

### Part 3. Preparation of 2-[2-(1H-Benzoimidazol-2-yl)-ethyl]-5-(2-methoxy-phenyl)-4-p-tolyl-2,4-dihydro-[1,2,4]triazole-3-thione

A mixture of the propionimidic acid ethyl ester and benzene-1,2-diamine (0.227 g, 2.1 mmol) in ethanol (10 mL) was stirred and heated at 60° C. overnight. The solvent was removed under reduce pressure, the residue was partitioned between ethyl acetate (20 mL) and saturated aqueous sodium bicarbonate (10 mL). The organic layer was dried over sodium sulfate, filtered, and the solvent removed under reduce pressure. Flash chromatography (SiO₂, 1:1 ethyl acetate/dichloromethane) gave a colorless oil. The oil was dissolved in methanol (2 mL) and treated with ethereal 2M HCl (10 mL). The solvent was removed under reduce pressure to provide the mono HCl salt of Compound E-3 (0.33 g) as a white solid.

### Compound E-4

### 5-(2-Methoxy-phenyl)-2-(2-pyridin-4-yl-ethyl)-4-p-tolyl-2,4-dihydro-[1,2,4]triazole-3-thione

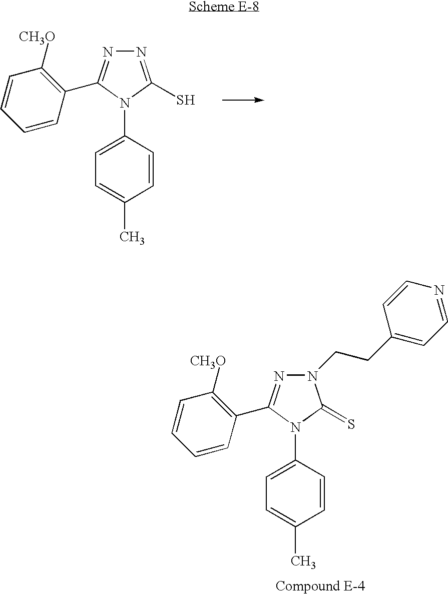

### Part 1. Preparation of 5-(2-Methoxy-phenyl)-2-(2-pyridin-4-yl-ethyl)-4-p-tolyl-2,4-dihydro-[1,2,4]triazole-3-thione

A mixture of 5-(2-methoxy-phenyl)-4-p-tolyl-2,4-dihydro-[1,2,4]triazole-3-thione (0.15 g, 0.50 mmol) in ethanol (10 mL) was stirred and 4-vinylpyridine (0.15 g, 1.0 mmol) was added. The mixture was heated overnight at refluxed then cooled. The cooled mixture was concentrated under vacuum and the residue diluted with ethyl acetate. The organics were washed with water (10 mL) and brine (10 mL) and dried over sodium sulfate, filtered and the solvent removed under reduce pressure to give a viscous yellow oil. Flash chromatography (SiO₂, 20% ethyl acetate/hexane) gave 5-(2-methoxy-phenyl)-2-(2-pyridin-4-yl-ethyl)-4-p-tolyl-2,4-dihydro-[1,2,4]triazole-3-thione (0.04 g, 0.09 mmol) as a white solid.

### Compound E-5

### 1-(4-Chloro-phenyl)-3-[3-(2-methoxy-phenyl)-5-thioxo-4-p-tolyl-4,5-dihydro-[1,2,4]triazol-1-yl]-propan-1-one

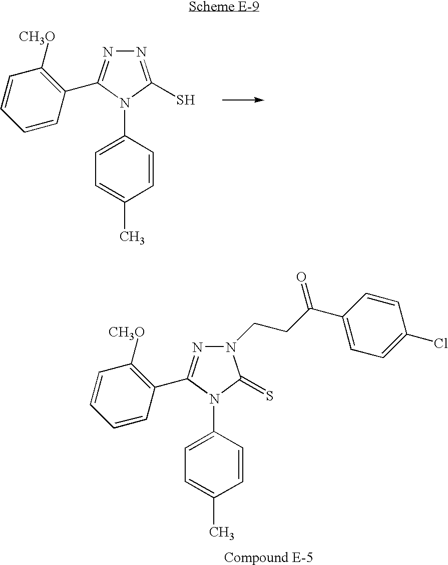

### Part 1. Preparation of 1-(4-Chloro-phenyl)-3-[3-(2-methoxy-phenyl)-5-thioxo-4-p-tolyl-4,5-dihydro-[1,2,4]triazol-1-yl]-propan-1-one

To a solution of 5-(2-methoxy-phenyl)-4-p-tolyl-4H-[1,2,4]triazole-3-thiol (0.45 g, 1.5 mmol) in DMF (10 mL) was added a 1M solution of lithium bis(trimethylsilyl)amide in THF (1.5 mL) and beta-4-dichloropropiophenone (0.30 g, 1.5 mmol) at room temperature. The mixture was heated at 60° C. for 1 hour and cooled to room temperature. The mixture was quenched with water and extracted with ethyl acetate. The organics were dried and concentrated under vacuum. The residue was purified by chromatography on silica (20% ethyl acetate in n-hexane) to give 1-(4-chloro-phenyl)-3-[3-(2-methoxy-phenyl)-5-thioxo-4-p-tolyl-4,5-dihydro-[1,2,4]triazol-1-yl]-propan-1-one (0.19 g, 0.41 mmol) as a white solid.

### EXAMPLE F-4

### Compound F-1

### 2-(2-Methoxy-phenyl)-1-p-tolyl-1H-imidazole-4-carboxylic acid ethyl ester (Compound 1 in Scheme F-5)

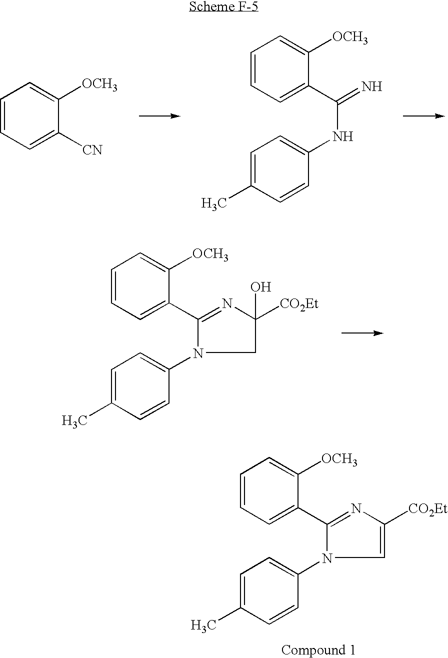

### Part 1. Preparation of 2-Methoxy-N-p-tolyl-benzamidine

To a solution of sodium bis(trimethylsilyl)amide in THF (9.9 mL, 1M solution, 9.9 mmol) was slowly added at room temperature a solution of p-toluidine (1 g, 9.3 mmol) in dry THF (5 mL). After the mixture was stirred for 20 minutes, a solution of 2-methoxybenzonitrile (1.32 g, 9.9 mmol) in dry THF (5 mL) was added. The reaction mixture was stirred for 4 hours and quenched with water. The mixture was extracted with ethyl acetate three times. The combined organic layers were washed with water, brine, dried over anhydrous Na₂SO₄, filtered and concentrated under vacuum to give 2-methoxy-N-p-tolyl-benzamidine as a red oil, which was used in the next step without further purification.

### Part 2. Preparation of 4-Hydroxy-2-(2-methoxy-phenyl)-1-p-tolyl-4,5-dihydro-11H-imidazole-4-carboxylic acid ethyl ester

A mixture of 2-methoxy-N-p-tolyl-benzamidine (340 mg, 1.5 mmol), NaHCO₃, (378 mg, 4.5 mmol) in THF/water (4/1: v/v, 10 mL) was heated at reflux. A solution of ethyl bromopyruvate (0.19 mL, 1.5 mmol) in THF (2 mL) was added over 5 minutes. The reaction mixture was refluxed for 2 hours, cooled to room temperature, extracted with ethyl acetate three times. The combined organic layers were washed with water, brine, dried over anhydrous Na₂SO₄, filtered and concentrated under vacuum to give 4-hydroxy-2-(2-methoxy-phenyl)-1-p-tolyl-4,5-dihydro-1H-imidazole-4-carboxylic acid ethyl ester as a brown solid and used without purification in the next step.

### Part 3. Preparation of 2-(2-Methoxy-phenyl)-1-p-tolyl-1H-imidazole-4-carboxylic acid ethyl ester

To the flask of 4-hydroxy-2-(2-methoxy-phenyl)-1-p-tolyl-4,5-dihydro-1H-imidazole-4-carboxylic acid ethyl ester (5 g, 14.1 mmol) in dry toluene (50 mL) was added p-toluenesulfonic acid (268 mg, 1.4 mmol). The resulting mixture was refluxed until starting material was consumed. The solvent was removed under vacuum and the resulting residue was partitioned between ethyl acetate and saturated aqueous NaHCO₃. The aqueous layer was extracted with ethyl acetate. The combined organic layers were washed with water, brine, dried over anhydrous Na₂SO₄, filtered, concentrated under vacuum and chromatography on(SiO₂, 50% ethyl acetate in hexanes) provided 2-(2-methoxy-phenyl)-1-p-tolyl-1H-imidazole-4-carboxylic acid ethyl ester (4.5 g, 13.4 mmol) as a solid.

### Compound F-2

### 2-(2-Methoxy-phenyl)-1-p-tolyl-1H-imidazole-4-carboxylic acid (4-fluoro-phenyl)-methyl-amide (Compound 2 in scheme F-6)

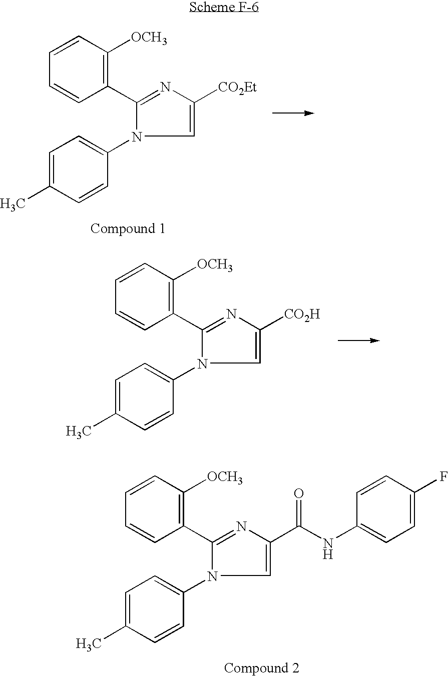

### Part 1. Preparation of 2-(2-Methoxy-phenyl)-1-p-tolyl-1H-imidazole-4-carboxylic acid

To a solution of 2-(2-methoxy-phenyl)-1-p-tolyl-1H-imidazole-4-carboxylic acid ethyl ester (4.5 g, 13.4 mmol) in methanol (10 mL) was added aqueous 2N NaOH (10 mL). The mixture was refluxed for 1 hour and cooled to room temperature. The solvents were partially removed under reduced pressure. The residue was acidified to pH 3, extracted with methylene chloride three times. The combined organic layers were washed with water, brine, dried over anhydrous Na₂SO₄, filtered and concentrated under vacuum to 2-(2-methoxy-phenyl)-1-p-tolyl-1H-imidazole-4-carboxylic acid (4.1 g, 13.4 mmol) as a solid.

### Part 2. Preparation of 2-(2-Methoxy-phenyl)-1-p-tolyl-1H-imidazole-4-carboxylic acid (4-fluoro-phenyl)-amide

To the flask containing 2-(2-methoxy-phenyl)-1-p-tolyl-1H-imidazole-4-carboxylic acid (740 mg, 2.4 mmol), 4-fluoroaniline (0.23 mL, 2.4 mmol) and 1-[3-(dimethylamino)propyl]-3-ethylcarbodiimide hydrochloride (920 mg, 4.8 mmol) was added pyridine (10 mL). The mixture was stirred at room temperature for 1 hour and the volatile organics were removed. The residue was partitioned between methylene chloride and water. The combined organic layers were washed with water, brine, dried over anhydrous Na₂SO₄, filtered and concentrated under vacuum. Chromatography (SiO₂, 30% ethyl acetate in hexanes) afforded 2-(2-methoxy-phenyl)-1-p-tolyl-1H-imidazole-4-carboxylic acid (4-fluoro-phenyl)-amide (900 mg, 2.2 mmol) as a solid.

### Compound F-3

### (4-Fluoro-phenyl)-[2-(2-methoxy-phenyl)-1-p-tolyl-1H-imidazole-4-ylmethyl]-amine (Compound 3 in Scheme F-7)

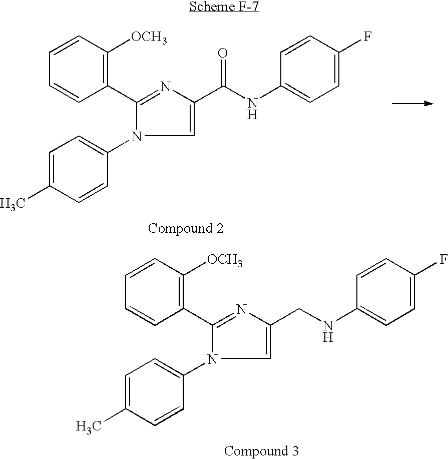

To a solution of 2-(2-methoxy-phenyl)-1-p-tolyl-1H-imidazole-4-carboxylic acid (4-fluoro-phenyl)-amide (250 mg, 0.62 mmol) in toluene (6 mL) was added at room temperature borane dimethylsulfide complex THF solution (1.25 mL, 2M solution, 2.5 mL). The mixture was refluxed overnight. To the cooled reaction mixture was added aqueous 1N HCl. The mixture was refluxed for 30 minutes and cooled to room temperature. The solvents were partially removed under reduced pressure. The residue was neutralized with aqueous 1N NaOH and extracted with methylene chloride three times. The organics were washed with water, brine, dried over anhydrous Na₂SO₄, filtered and concentrated under vacuum. Chromatography (SiO₂, 30% ethyl acetate in hexanes) afforded (4-fluoro-phenyl)-[2-(2-methoxy-phenyl)-1-p-tolyl-1H-imidazole-4-ylmethyl]-amine (210 mg, 0.54 mmol) as an oil.

### Compound F-4

### of 2-(2-Methoxy-phenyl)-1-p-tolyl-1H-imidazole-4-carboxylic acid methoxy-methyl-amide (Compound 4 in Scheme F-8)

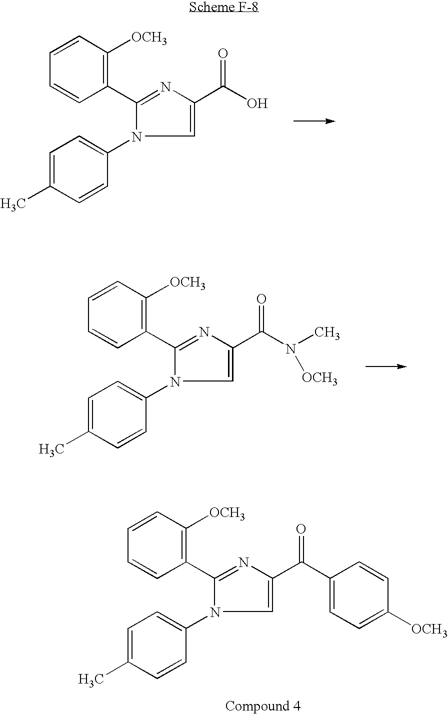

### Part 1. Preparation of 2-(2-Methoxy-phenyl)-1-p-tolyl-1H-imidazole-4-carboxylic acid methoxy-methyl-amide

To a solution of 2-(2-methoxy-phenyl)-1-p-tolyl-1H-imidazole-4-carboxylic acid (200 mg, 0.65 mmol) in methylene chloride (5 mL) was added (dimethylamino)propyl]-3-ethylcarbodiimide hydrochloride (250 mg, 1.3 mmol), triethylamine (0.18 mL, 1.3 mmol) and N,O-dimethylhydroxylamine hydrochloride (63 mg, 0.65 mmol). The mixture was stirred overnight. The mixture was applied to partition between methylene chloride and saturated aqueous NaHCO₃. The combined organic layers were washed with water, brine, dried over anhydrous Na₂SO₄, filtered and concentrated under vacuum. Cohromatography (SiO₂, ethyl acetate) afforded 2-(2-methoxy-phenyl)-1-p-tolyl-1H-imidazole-4-carboxylic acid methoxy-methyl-amide (220 mg, 0.63 mmol) as an oil.

### Part 2. Preparation of (4-Methoxy-phenyl)-[2-(2-methoxy-phenyl)-1-p-tolyl-1H-imidazole-4-yl]-methanone

To a solution of 2-(2-methoxy-phenyl)-1-p-tolyl-1H-imidazole-4-carboxylic acid methoxy-methyl-amide (110 mg, 0.31 mmol) in THF (2 mL) was slowly added 4-methoxyphenylmagnesium bromide THF solution (0.63 mL, 0.5 M solution, 0.31 mmol). The mixture was stirred overnight. Additional 0.5M 4-methoxyphenylmagnesium bromide in THF (0.63 mL, 0.31 mmol) was added and the mixture was stirred for 3 hours and quenched with water. The mixture was extracted with ethyl acetate. The combined organic layers were washed with water, brine, dried over anhydrous Na₂SO₄, filtered and concentrated under vacuum. Reversed phase liquid chromatography followed by plate chromatography (SiO₂, 50% ethyl acetate in hexanes) afforded (4-methoxy-phenyl)-[2-(2-methoxy-phenyl)-1-p-tolyl-1H-imidazole-4-yl]-methanone (9.1 mg, 0.04 mmol) as a foam.

### Compound F-5

### (4-Methoxy-phenyl)-2-(2-methoxy-phenyl)-1-p-tolyl-1H-imidazole (Compound 5 in scheme F-9)

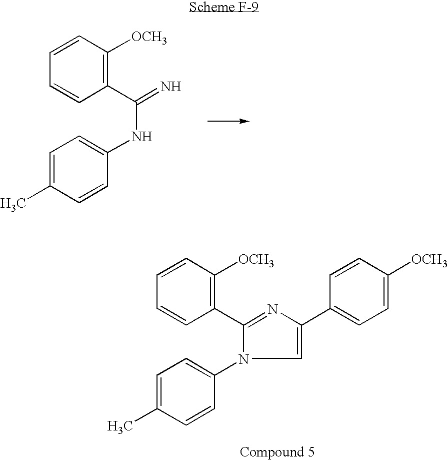

To a solution of 2-methoxy-N-p-tolyl-benzamidine (230 mg, 0.96 mmol) in acetonitrile (5 mL) was added NaHCO₃ (242 mg, 2.88 mmol). The mixture was heated to 50° C. A solution of 2-bromo-1-(4-methoxy-phenyl)-ethanone (220 mg, 0.96 mmol) in acetonitrile (2 mL) was added dropwise and the mixture was stirred at 50° C. for 30 minutes and refluxed for 3 hours. The solvent was removed, the residue was applied to column chromatography (SiO₂, 30% ethyl acetate in hexane) to give (4-methoxy-phenyl)-2-(2-methoxy-phenyl)-1-p-tolyl-1H-imidazole (320 mg, 0.86 mmol) as a solid.

### EXAMPLE G-4

Representative compounds of the formulae herein were evaluated for activity against calcium channel targets.

### Compound G-1

### [1-(4-Chloro-phenyl)-2-(4-fluoro-benzyl)-1H-imidazol-4-ylmethyl]-(4-fluoro-phenyl)-methyl-amine (Compound 1 in Scheme G-6)

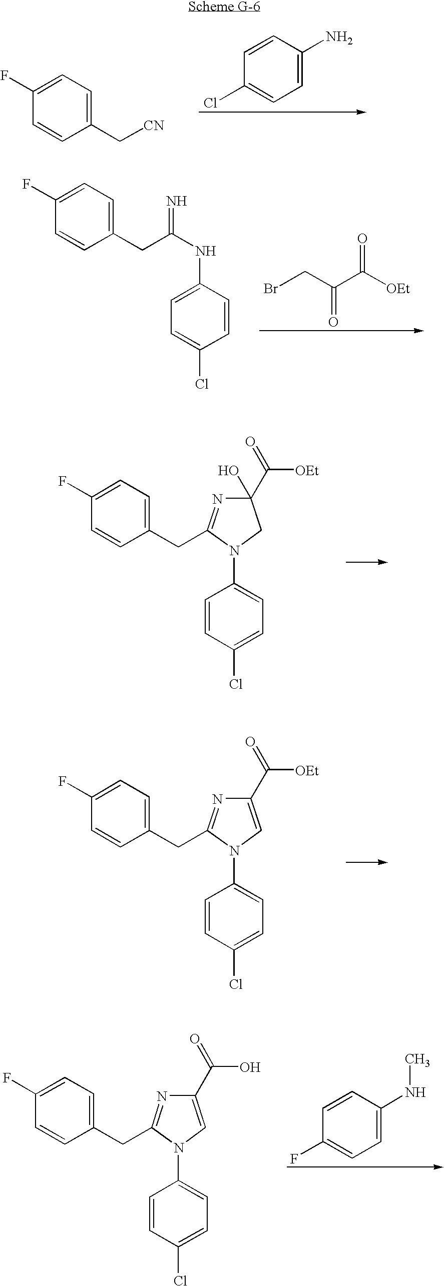

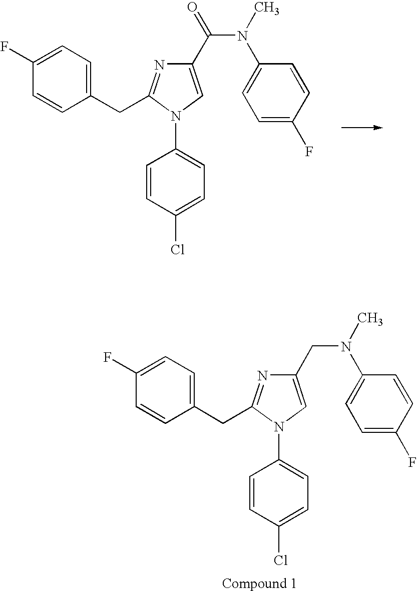

### Part G-1. Preparation of N-(4-Chloro-phenyl)-2-(4-fluoro-phenyl)-acetamidine

To 4-chloroaniline (54.0 g, 424 mmol) in toluene (1000 mL) at 0° C. was added, dropwise, trimethylaluminum (2.0 M in toluene, 200 mL, 400 mmol) and the reaction warmed to room temperature over 3 hours under nitrogen. A solution of 4-fluorophenylacetonitrile (31.8 g, 235 mmol) in toluene (20 mL) was added and the mixture heated at 80° C. overnight. The mixture was cooled to room temperature, treated with chloroform (200 ml) and SiO₂, the slurry stirred 1 hour and poured onto a plug of SiO₂. Elution with 5:10:85 ammonium hydroxide:methanol:methylene chloride gave upon concentration in vacuo a light brown solid. Recrystallization from ethyl acetate/hexanes gave N-(4-chloro-phenyl)-2-(4-fluoro-phenyl)-acetamidine (54.5 g, 207 mmol) as a white solid.

### Part G-2. Preparation of 1-(4-Chloro-phenyl)-2-(4-fluoro-benzyl)-4-hydroxy-4,5-dihydro-1H-imidazole-4-carboxylic acid ethyl ester

A solution of N-(4-chloro-phenyl)-2-(4-fluoro-phenyl)-acetamidine (6.2 g, 24 mmol), ethyl bromopyruvate (10.1 g, 52 mmol), and sodium hydrogen carbonate (8.9 g, 106 mmol) in THF (100 mL) and water (100 ml) was refluxed 4 hours. The mixture was cooled to room temperature and extracted with diethyl ether. The organics were dried and concentrated in vacuo to give a dark brown oil. Recrystallization from ethyl acetate/hexanes gave 1-(4-chloro-phenyl)-2-(4-fluoro-benzyl)-4-hydroxy-4,5-dihydro-1H-imidazole-4-carboxylic acid ethyl ester (1.8 g, 4.7 mmol) as a light brown solid.

### Part G-3. Preparation of 1-(4-Chloro-phenyl)-2-(4-fluoro-benzyl)-1H-imidazole-4-carboxylic acid ethyl ester

A solution of 1-(4-chloro-phenyl)-2-(4-fluoro-benzyl)-4-hydroxy-4,5-dihydro-1H-imidazole-4-carboxylic acid ethyl ester (1.8 g, 4.7 mmol) and p-toluenesulfonic acid monohydrate (0.2 g, 0.9 mmol) in toluene (20 mL) was refluxed 2 hours. The mixture was cooled to room temperature, solvent removed in vacuo, and the residue partitioned between water and ethyl acetate. The organics were dried and concentrated in vacuo to give a dark red oil which was purified by chromatography (SiO₂, 3% methanol in methylene chloride) to give 1-(4-chloro-phenyl)-2-(4-fluoro-benzyl)-1H-imidazole-4-carboxylic acid ethyl ester (1.6 g, 4.4 mmol).

### Part G-4. Preparation of 1-(4-Chloro-phenyl)-2-(4-fluoro-benzyl)-1H-imidazole-4-carboxylic acid

To a solution of 1-(4-chloro-phenyl)-2-(4-fluoro-benzyl)-1H-imidazole-4-carboxylic acid ethyl ester (1.6 g, 4.4 mmol) in 1,4-dioxane (15 mL) and water (15 ml) was added lithium hydroxide hydrate (0.4 g, 8.7 mmol) and the mixture was stirred at 40° C. for 1 hour. Most 1,4-dioxane was removed in vacuo, the residue taken up in ethyl acetate/water, the aqueous washed with ethyl acetate, acidified to pH 2 with aqueous 2 N HCl and the precipitated 1-(4-chloro-phenyl)-2-(4-fluoro-benzyl)-1H-imidazole-4-carboxylic acid (1 g, 2.7 mmol) collected by filtration as a the white HCl salt.

### Part G-5. Preparation of 1-(4-Chloro-phenyl)-2-(4-fluoro-benzyl)-1H-imidazole-4-carboxylic acid (4-fluoro-phenyl)-methyl-amide

A mixture of 1-(4-chloro-phenyl)-2-(4-fluoro-benzyl)-1H-imidazole-4-carboxylic acid (0.4 g, 1.3 mmol) and 1-(3-dimethylaminopropyl)-3-ethylcarbodiimide hydrochloride (0.3 g, 1.5 mmol) and 4-fluoro-N-methylaniline (0.2 g, 1.4 mmol) in methylene chloride (10 mL) was stirred at room temperature for 4 hours. Solvent was removed in vacuo, the residue taken up in water and extracted with ethyl acetate. The organics were dried, concentrated in vacuo, and the residue purified by chromatography (SiO₂, 3% methanol in methylene chloride) to give 1-(4-chloro-phenyl)-2-(4-fluoro-benzyl)-1H-imidazole-4-carboxylic acid (4-fluoro-phenyl)-methyl-amide (0.4 g, 0.9 mmol).

### Part G-6. Preparation of [1-(4-Chloro-phenyl)-2-(4-fluoro-benzyl)-1H-imidazol-4-ylmethyl]-(4-fluoro-phenyl)-methyl-amine

To a solution 1-(4-chloro-phenyl)-2-(4-fluoro-benzyl)-1H-imidazole-4-carboxylic acid (4-fluoro-phenyl)-methyl-amide (0.3 gm, 0.7 mmol) in THF (10 mL) at 0° C. was added borane-dimethylsulfide complex (2M in THF, 1.0 mL, 2.0 mmol) and the reaction heated at reflux overnight. The mixture was cooled and diluted with methanolic HCl (10 mL), heated at reflux for 1 hour, cooled, and concentrated in vacuo. The residue was diluted with saturated aqueous sodium bicarbonate and extracted with ethyl acetate. The organics were dried and concentrated under vacuum to give an oil. Purification by chromatography (SiO₂, 3% methanol in methylene chloride) gave an oil which was taken up in ethanol and treated with HCl in ether to give 1-(4-chloro-phenyl)-2-(4-fluoro-benzyl)-1H-imidazol-4-ylmethyl]-(4-fluoro-phenyl)-methyl-amine (0.1 g, 0.2 mmol) as the off-white HCl salt.

### Compound G-2

### 2-[1-(4-Chloro-phenyl)-2-(4-fluoro-benzyl)-1H-imidazol-4-ylmethyl]-1H-benzoimidazole (Compound 2 in scheme G-7)

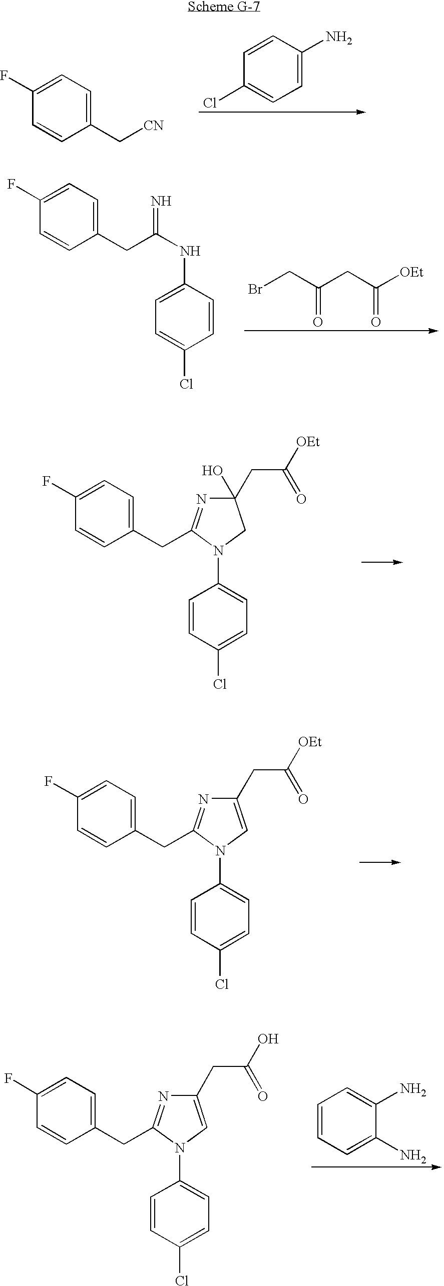

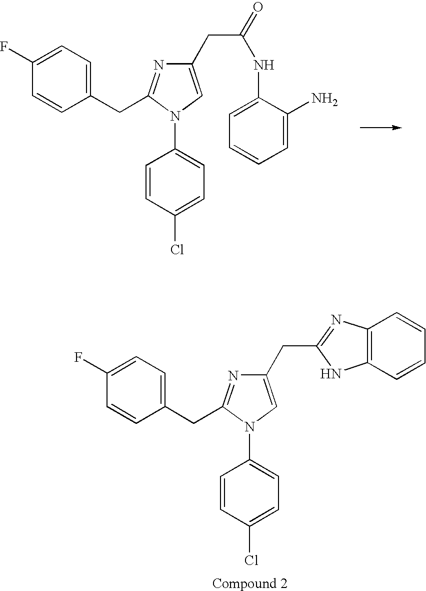

### Parts G-1-5. Preparation of N-(2-Amino-phenyl)-2-[1-(4-chloro-phenyl)-2-(4-fluoro-benzyl)-1H-imidazol-4-yl]-acetamide

N-(2-Amino-phenyl)-2-[1-(4-chloro-phenyl)-2-(4-fluoro-benzyl)-1H-imidazol-4-yl]-acetamide was made as in Scheme 6 substituting ethyl 4-bromoacetoacetate for ethyl bromopyruvate in Part 2 and 1,2-phenylenediamine for 4-fluoro-N-methylaniline in Part G-5.

### Part G-6. Preparation of 2-[1-(4-Chloro-phenyl)-2-(4-fluoro-benzyl)-1H-imidazol-4-ylmethyl]-1H-benzoimidazole

A solution of N-(2-amino-phenyl)-2-[1-(4-chloro-phenyl)-2-(4-fluoro-benzyl)-1H-imidazol-4-yl]-acetamide (3.8 g, 8.7 mmol) in glacial acetic acid (10 mL) was heated at 70° C. for 0.5 hours. The mixture was cooled, added dropwise to saturated aqueous sodium hydrogen carbonate, the pH adjusted to ˜14 with sodium hydroxide, extracted with ethyl acetate, and the organics dried and concentrated in vacuo to give an oil. Treatment with HCl in ether followed by recrystallization from methanol/ether gave 2-[1-(4-chloro-phenyl)-2-(4-fluoro-benzyl)-1H-imidazol-4-ylmethyl]-1H-benzoimidazole (2.0 g, 4.8 mmol) as the white HCl salt.

### EXAMPLE H-4

Representative compounds of the formulae herein are evaluated for activity against calcium channel targets

### Compound H-1

### 1-(4-Chloro-phenyl)-2-{[(4-fluoro-phenyl)-methyl-amino]-methyl}-1H-imidazole-4-carboxylic acid ethyl ester

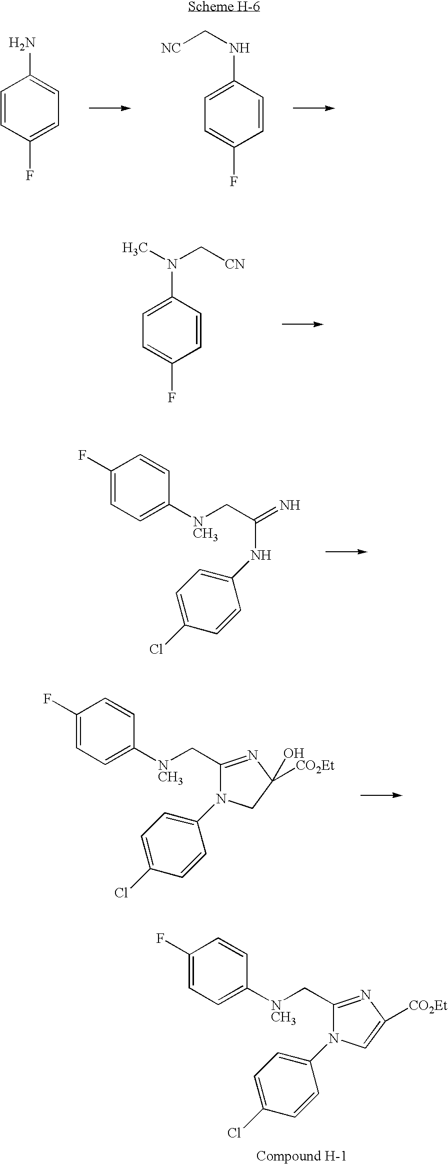

### Part H-1. Preparation of (4-Fluoro-phenylamino)-acetonitrile

To a solution of 4-fluoroaniline (20.0 g, 180 mmol) in glacial acetic acid (250 mL) was added portion wise paraformaldehyde (14.06 g) and potassium cyanide (14.06 g, 216 mmol) at 0° C. The mixture was allowed to stir at room temperature overnight and cooled. The mixture was neutralized with saturated aqueous sodium bicarbonate and extracted with ethyl acetate. The organics were dried and concentrated under vacuum. The resulting residue was purified by chromatography (SiO₂, 10% ethyl acetate in n-hexane to give (4-fluoro-phenylamino)-acetonitrile (22.9 g, 153 mmol) as a yellow oil.

### Part H-2. Preparation of [(4-Fluoro-phenyl)-methyl-amino]-acetonitrile

To a slurry of (4-fluoro-phenylamino)-acetonitrile (22.9 g, 153 mmol) and cesium carbonate (74.8 g, 229 mmol) in THF (200 mL) was added iodomethane (10.5 mL, 16 8 mmol). The mixture was stirred for 3 hours at 40° C., cooled and quenched with water and extracted with ethyl acetate. The organics were dried and concentrated under vacuum to give [(4-fluoro-phenyl)-methyl-amino]-acetonitrile (22.3 g, 136 mmol) as an oil.

### Part H-3. Preparation of N-(4-Chloro-phenyl)-2-[(4-fluoro-phenyl)-methyl-amino]-acetamidine

To a solution of 4-chloroaniline (1.4 g, 11.0 mmol) in toluene (50 mL) was added trimethylaluminum (2M in toluene; 5.3 mL, 10.4 mmol) at 0° C. under a nitrogen blanket. The slurry was allowed to stir for 1 hour and added to a solution of [(4-fluoro-phenyl)-methyl-amino]-acetonitrile (1.0 g, 6.2 mmol) at room temperature. The mixture was heated at 80° C. overnight, cooled and quenched with a slurry of silica/chloroform mixture. The resulting mixture was filtered over a short bed of silica and washed with 10% methanol in methylene chloride. The combined fractions gave N-(4-chloro-phenyl)-2-[(4-fluoro-phenyl)-methyl-amino]-acetamidine (1.21 g, 4.18 mmol) as yellow oil.

### Part H-4. Preparation of 1-(4-Chloro-phenyl)-2-{[(4-fluoro-phenyl)-methyl-amino]-methyl}-4-hydroxy-4,5-dihydro-1H-imidazole-4-carboxylic acid ethyl ester

To a solution of N-(4-chloro-phenyl)-2-[(4-fluoro-phenyl)-methyl-amino]-acetamidine (1.21 g, 4.18 mmol) in THF (40 mL) was added sodium bicarbonate (0.70 g, 8.36 mmol) in water (10 mL) followed by slow addition of ethyl bromopyruvate (1.22 gm, 6.27 mmol) at 40° C. After addition, the reaction was heated at 40° C. for 2 hours and cooled. The mixture was diluted with water and extracted with ethyl acetate. The organics were dried and concentrated under vacuum. The resulting residue was purified by chromatography (SiO₂, 30% ethyl acetate in n-hexane) to give 1-(4-chloro-phenyl)-2-{[(4-fluoro-phenyl)-methyl-amino]-methyl}-4-hydroxy-4,5-dihydro-1H-imidazole-4-carboxylic acid ethyl ester (0.74 g, 1.84 mmol) as a dark oil.

### Part H-5. Preparation of 1-(4-Chloro-phenyl)-2-{[(4-fluoro-phenyl)-methyl-amino]-methyl}-1H-imidazole-4-carboxylic acid ethyl ester

A mixture of 1-(4-chloro-phenyl)-2-{[(4-fluoro-phenyl)-methyl-amino]-methyl}-4-hydroxy-4,5-dihydro-1H-imidazole-4-carboxylic acid ethyl ester (0.74 gm, 1.84 mmol) and p-toluenesulfonic acid monohydrate (0.1 gm) in toluene (20 mL) was heated at reflux for 1 hour. The mixture was cooled, quenched with water and extracted with ethyl acetate. The organics were dried and concentrated under vacuum. The resulting residue was purified by chromatography (SiO₂, 15% acetone in n-hexane) to give 1-(4-chloro-phenyl)-2-{[(4-fluoro-phenyl)-methyl-amino]-methyl}-1H-imidazole-4-carboxylic acid ethyl ester (0.63 g, 1.62 mmol) as a white solid.

### Compound H-2 and H-3

### 1-(4-Chloro-phenyl)-2-{[(4-fluoro-phenyl)-methyl-amino]-methyl}-1H-imidazole-4-carboxylic acid phenylamide and [1-(4-Chloro-phenyl)-4-phenylaminomethyl-1H-imidazol-2-ylmethyl]-(4-fluoro-phenyl)-methyl-amine

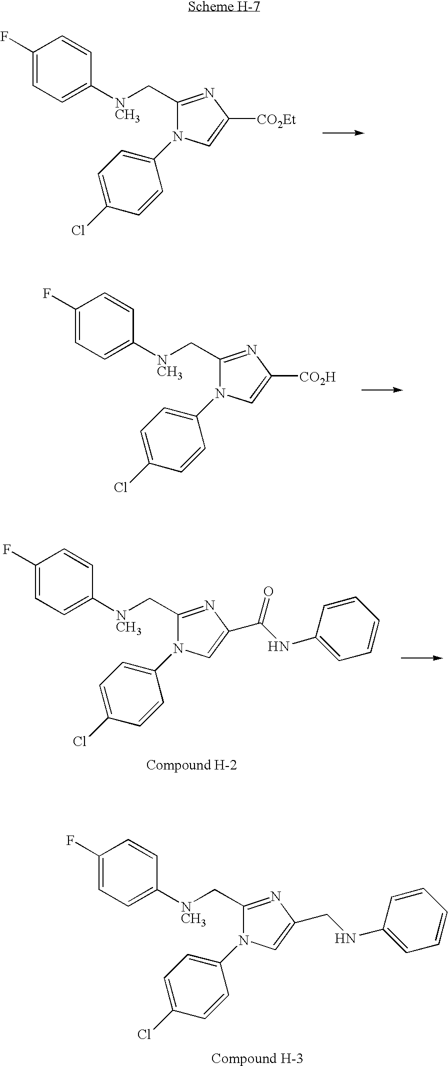

### Part H-1. Preparation of 1-(4-Chloro-phenyl)-2-{[(4-fluoro-phenyl)-methyl-amino]-methyl}-1H-imidazole-4-carboxylic acid

A solution of 1-(4-chloro-phenyl)-2-{[(4-fluoro-phenyl)-methyl-amino]-methyl}-1H-imidazole-4-carboxylic acid ethyl ester (0.63 g, 1.62 mmol) and lithium hydroxide hydrate (0.14 g, 3.24 mmol) in a methanol/water mixture (2:1/v:v) was heated at 50° C. for 1 hour and cooled. The reaction mixture was concentrated under vacuum and diluted with aqueous 6N HCl until a pH of 6.5 was attained. The aqueous layer was extracted with ethyl acetate and the organics were dried, concentrated under vacuum to give 1-(4-chloro-phenyl)-2-{[(4-fluoro-phenyl)-methyl-amino]-methyl}-1H-imidazole-4-carboxylic acid (0.41 g, 1.15 mmol) as a white solid.

### Part H-2. Preparation of 1-(4-Chloro-phenyl)-2-{[(4-fluoro-phenyl)-methyl-amino]-methyl}-1H-imidazole-4-carboxylic acid phenylamide

A mixture of 1-(4-chloro-phenyl)-2-{[(4-fluoro-phenyl)-methyl-amino]-methyl}-1H-imidazole-4-carboxylic acid (0.36 g, 1.00 mmol) and 1-[3-(dimethylamino)propyl]-3-ethylcarbodiimide hydrochloride (0.28 g, 1.5 mmol) and aniline (0.09 g, 1 mmol) in pyridine (4 mL) was heated at 40° C. overnight. The mixture was cooled, quenched with water and extracted with ethyl acetate. The organics were dried, concentrated under vacuum to 1-(4-chloro-phenyl)-2-{[(4-fluoro-phenyl)-methyl-amino]-methyl}-1H-imidazole-4-carboxylic acid phenylamide (0.31 g, 0.71 mmol) as a solid.

### Part H-3. Preparation of [1-(4-Chloro-phenyl)-4-phenylaminomethyl-1H-imidazol-2-ylmethyl]-(4-fluoro-phenyl)-methyl-amine

To a solution 1-(4-chloro-phenyl)-2-{[(4-fluoro-phenyl)-methyl-amino]-methyl}-1H-imidazole-4-carboxylic acid phenylamide (0.26 g, 0.6 mmol) in THF (15 mL) was added borane-dimethylsulfide complex (2M in THF; 0.9 mL) and allowed to stir overnight at reflux. The mixture was cooled and diluted with methanolic HCl (10 mL). The mixture was heated again at reflux for 1 hour, cooled and concentrated under vacuum to give a residue. The residue was diluted with saturated aqueous sodium bicarbonate and extracted with ethyl acetate. The organics were dried and concentrated under vacuum to give a solid. Purification by chromatography (SiO₂, 40% acetone in n-hexane) gave [1-(4-chloro-phenyl)-4-phenylaminomethyl-1H-imidazol-2-ylmethyl]-(4-fluoro-phenyl)-methyl-amine (0.07 g, 0.17 mmol) as a white solid.

### Compound H-4

### [4-(1H-Benzoimidazol-2-ylmethyl)-1-(4-chloro-phenyl)-1H-imidazol-2-ylmethyl]-(4-fluoro-phenyl)-methyl-amine

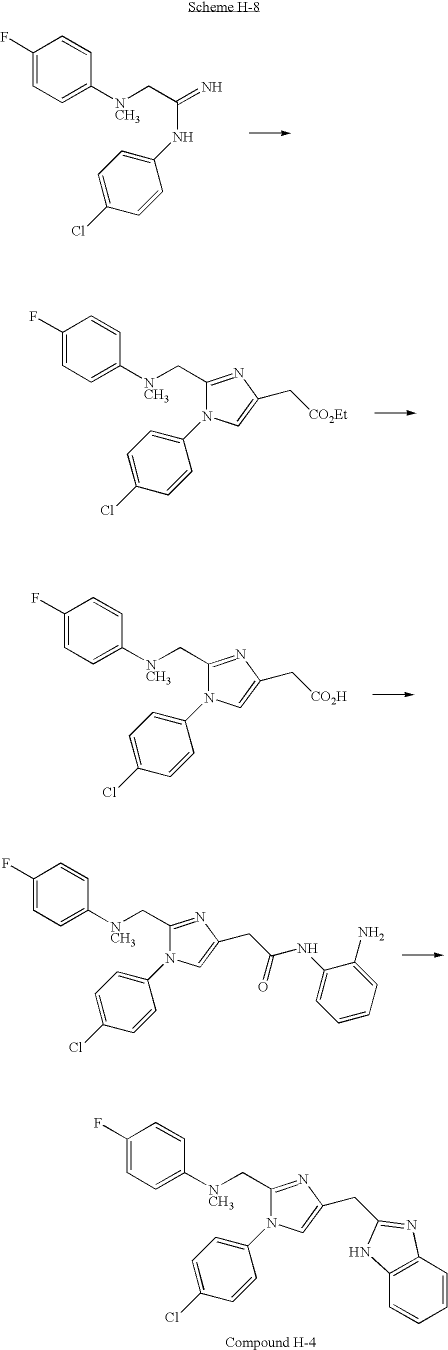

### Part H-1. Preparation of (1-(4-Chloro-phenyl)-2-{[(4-fluoro-phenyl)-methyl-amino]-methyl}-1H-imidazol-4-yl)-acetic acid ethyl ester

[1-(4-chloro-phenyl)-4-phenylaminomethyl-1H-imidazol-2-ylmethyl]-(4-fluoro-phenyl)-methyl-amine (1 eq.) and potassium hydrogencarbonate (3 eq) is suspended in acetonitrile. The suspension is heated to 50° C. and 4-bromo-3-oxo-butyric acid ethyl ester (1.5 eq.) in acetonitrile is added slowly dropwise. The reaction mixture is refluxed for 2 hours and cooled. The resulting residue is purified by chromatography (SiO₂, ethyl acetate in hexane to give [1-(4-chloro-phenyl)-2-(2-methoxy-phenyl)-1H-imidazol-4-yl]-acetic acid ethyl ester.

### Part H-2. Preparation of (1-(4-Chloro-phenyl)-2-{[(4-fluoro-phenyl)-methyl-amino]-methyl}-1H-imidazol-4-yl)-acetic acid

To a solution of (1-(4-chloro-phenyl)-2-{[(4-fluoro-phenyl)-methyl-amino]-methyl}-1H-imidazol-4-yl)-acetic acid ethyl ester (1 eq.) in THF is added 1N aqueous sodium hydroxide (5 eq). The mixture is stirred for 1 hour at 70° C. and cooled. The reaction is diluted with water and the aqueous layer the pH is adjusted to 6 using 6N aqueous sodium hydroxide. The aqueous phase is extracted with ethyl acetate, washed with water, dried and concentrated under vacuum to give (1-(4-chloro-phenyl)-2-{[(4-fluoro-phenyl)-methyl-amino]-methyl}-1H-imidazol-4-yl)-acetic acid.

### Part H-3. Preparation of N-(2-Amino-phenyl)-2-(1-(4-chloro-phenyl)-2-{[(4-fluoro-phenyl)-ethyl-amino]-methyl}-1H-imidazol-4-yl)-acetamide

A mixture of (1-(4-chloro-phenyl)-2-{[(4-fluoro-phenyl)-methyl-amino]-methyl}-1H-imidazol-4-yl)-acetic acid (1 eq.) and 1-(3-dimethylaminopropyl)-3-ethylcarbodiimide hydrochloride (1.2 eq) and 1,2-phenylenediamine (1 eq.) in pyridine is stirred at room temperature overnight. The solvent is removed under vacuum, the resulting residue is diluted with water and extracted with ethyl acetate. The organics will be dried, concentrated and the residue is purified by chromatography on silica gel (methanol in methylene chloride) to give N-(2-amino-phenyl)-2-(1-(4-chloro-phenyl)-2-{[(4-fluoro-phenyl)-ethyl-amino]-methyl}-1H-imidazol-4-yl)-acetamide.

### Part H-4. Preparation of [4-(1H-Benzoimidazol-2-ylmethyl)-1-(4-chloro-phenyl)-1H-imidazol-2-ylmethyl]-(4-fluoro-phenyl)-methyl-amine

A solution of N-(2-amino-phenyl)-2-(1-(4-chloro-phenyl)-2-{[(4-fluoro-phenyl)-ethyl-amino]-methyl}-1H-imidazol-4-yl)-acetamide (1 eq.) in glacial acetic acid is to be heated at 70° C. for 30 minutes. The mixture is cooled and saturated aqueous sodium bicarbonate solution is added. The pH is adjusted to 7 with sodium hydroxide pellets and the aqueous layer extracted with ethyl acetate. The organics are dried and concentrated under vacuum to give a residue. The residue is treated with HCl in ether to give 2-[1-(4-chloro-phenyl)-2-(2-methoxy-phenyl)-1H-imidazol-4-ylmethyl]-1H-benzoimidazole (0.44 gm, 0.98 mmol) as an HCl salt.

### EXAMPLE J-4

### Compound 1

### [1-(4-{2-[1-(4-Chloro-phenyl)-2-(2-methoxy-phenyl)-1H-imidazol-4-yl]-ethyl}-(4-fluoro-phenyl)-methyl-amine

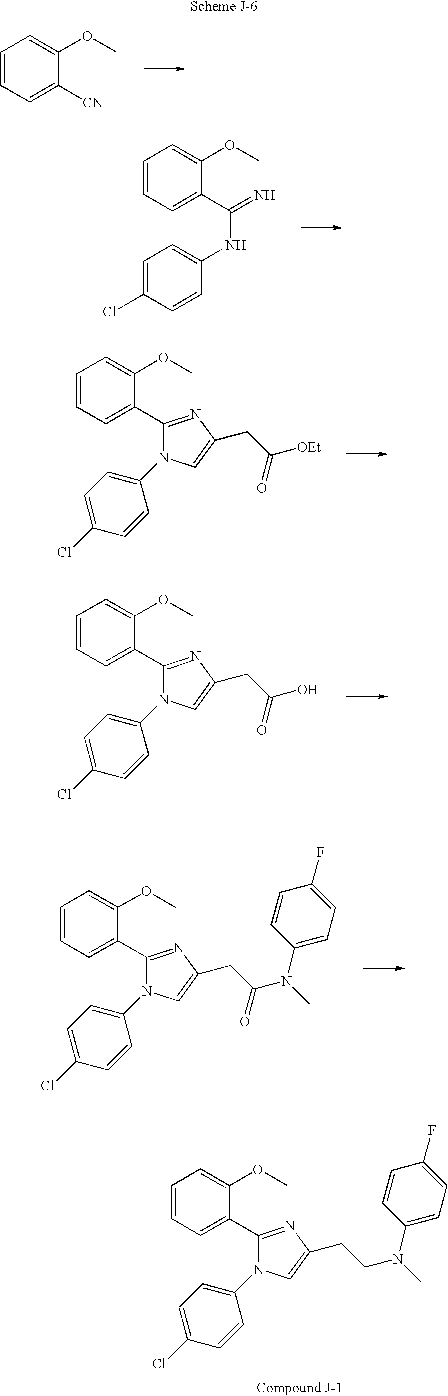

### Part J-1. Preparation of N-(4-Chloro-phenyl)-2-methoxy-benzamidine

To a solution of 4-chloroaniline (25 g, 197 mmol) in THF (250 mL) at 0° C. was added in a dropwise fashion a 1M solution of sodium bis(trimethylsilyl)amide in THF (207 mL, 1.06 eq) over a period of 30 to 60 minutes. After the addition was complete, a solution of 2-methoxy benzonitrile (27.6 g, 209 mmol) in THF (125 mL) was added dropwise over a period of 15 to 30 minutes at room temperature and stirred at room temperature for 1 hour. The solvent was removed under reduced pressure and the residue was partitioned between water and ethyl acetate. The combined organics were washed with brine, dried over sodium sulfate, filtered and the solvent was removed under reduce pressure to give dark oil that solidified upon standing. Titration with hexane and a minimal amount of ethyl acetate gave after filtration N-(4-chloro-phenyl)-2-methoxy-benzamidine (34 g, 131 mmol) as a grey solid.

### Part J-2. Preparation of [1-(4-Chloro-phenyl)-2-(2-methoxy-phenyl)-1H-imidazol-4-yl]-acetic acid ethyl ester

A 50° C. mixture of N-(4-chloro-phenyl)-2-methoxy-benzamidine (9 g, 34.6 mmol) and potassium hydrogencarbonate (10.38 g, 103.8 mmol, 3 eq) in acetonitrile (100 mL) was treated with a solution of 4-bromo-3-oxo-butyric acid ethyl ester (10 g, 48 mmol) in acetonitrile (50 mL) dropwise over 30 minutes. The reaction mixture was brought to reflux for 2 hours, cooled and filtered. Under vacuum the solvent was removed from the filtrate to give a dark oil. Flash chromatography (SiO₂, 50% ethyl acetate in hexane) gave [1-(4-chloro-phenyl)-2-(2-methoxy-phenyl)-1H-imidazol-4-yl]-acetic acid ethyl ester (16 g, 17 mmol) as a dark, viscous oil.

### Part J-3. Preparation of [1-(4-Chloro-phenyl)-2-(2-methoxy-phenyl)-1H-imidazol-4-yl]-acetic acid

To a solution of [1-(4-chloro-phenyl)-2-(2-methoxy-phenyl)-1H-imidazol-4-yl]-acetic acid ethyl ester (1.5 g, 4.04 mmol) in THF (40 mL) was added aqueous 1N sodium hydroxide (12 mL) and the mixture allowed to stir for 1 hour at 70° C. and cooled. The reaction was quenched with water and adjusted to pH 6 with aqueous 6N sodium hydroxide and extracted with ethyl acetate. The combined organics were washed with water, dried and concentrated under vacuum to give [1-(4-chloro-phenyl)-2-(2-methoxy-phenyl)-1H-imidazol-4-yl]-acetic acid (0.49 g, 1.43 mmol) as a white solid.

### Part J-4. Preparation of 2-[1-(4-Chloro-phenyl)-2-(2-methoxy-phenyl)-1H-imidazol-4-yl]-N-(4-fluoro-phenyl)-N-methyl-acetamide

A mixture of [1-(4-chloro-phenyl)-2-(2-methoxy-phenyl)-1H-imidazol-4-yl]-acetic acid (0.25 g, 0.73 mmol) and 1-(3-dimethylaminopropyl)-3-ethylcarbodiimide hydrochloride (0.28 g, 1.46 mmol) and 4-fluoro-N-methylaniline (0.082 mL, 0.73 mmol) in pyridine (3 mL) was stirred at room temperature overnight. The solvent was removed in vacuo, the residue diluted with water and extracted with ethyl acetate. The organics were dried, concentrated under reduced pressure and the residue purified by chromatography (SiO₂, 3% methanol in methylene chloride) to give 2-[1-(4-chloro-phenyl)-2-(2-methoxy-phenyl)-1H-imidazol-4-yl]-N-(4-fluoro-phenyl)-N-methyl-acetamide (0.16 g, 0.36 mmol) as an oil.

### Part J-5. Preparation of [1-(4-{2-[1-(4-Chloro-phenyl)-2-(2-methoxy-phenyl)-1H-imidazol-4-yl]-ethyl}-(4-fluoro-phenyl)-methyl-amine

To a solution of 2-[1-(4-chloro-phenyl)-2-(2-methoxy-phenyl)-1H-imidazol-4-yl]-N-(4-fluoro-phenyl)-N-methyl-acetamide (0.07 g, 0.16 mmol) in toluene (5 mL) at 0° C. was added borane-dimethylsulfide complex (2M in THF, 0.16 mL, 0.31 mmol) and the reaction heated at reflux overnight. The mixture was cooled and diluted with methanolic HCl (3 mL), heated at reflux for 1 hour, cooled and concentrated under vacuum. The residue was diluted with saturated aqueous sodium bicarbonate and extracted with ethyl acetate. The organics were dried, and concentrated under vacuum to give a white solid. The solid was taken up in methanol and treated with HCl in ether to give [1-(4-{2-[1-(4-chloro-phenyl)-2-(2-methoxy-phenyl)-1H-imidazol-4-yl]-ethyl}-(4-fluoro-phenyl)-methyl-amine (0.06 g, 0.013 mmol) as a white solid.

### Compound J-2

### 2-[1-(4-Chloro-phenyl)-2-(2-methoxy-phenyl)-1H-imidazol-4-ylmethyl]-1H-benzoimidazole

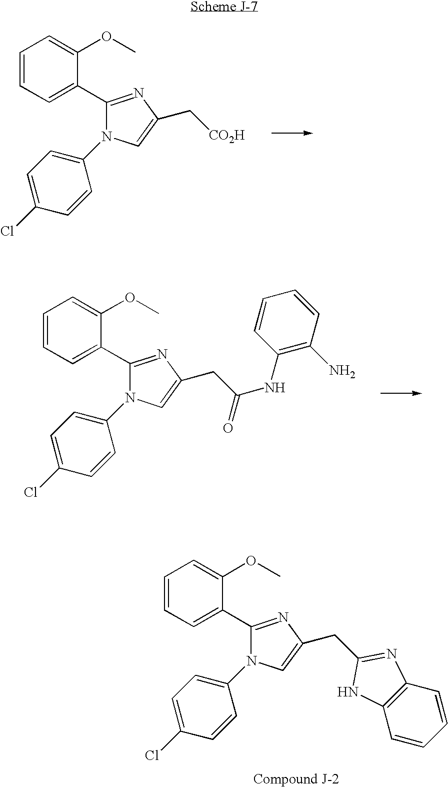

### Part J-1. Preparation of N-(2-Amino-phenyl)-2-[1-(4-chloro-phenyl)-2-(2-methoxy-phenyl)-1H-imidazol-4-yl]-acetamide

A mixture of [1-(4-chloro-phenyl)-2-(2-methoxy-phenyl)-1H-imidazol-4-yl]-acetic acid (0.87 g, 2.56 mmol) and 1-(3-dimethylaminopropyl)-3-ethylcarbodiimide hydrochloride (0.73 g, 3.83 mmol) and 1,2-phenylenediamine (0.28 g, 2.56 mmol) in pyridine (5 mL) was stirred at room temperature overnight. The solvent was removed in vacuo and was treated with water and made basic with saturated aqueous sodium bicarbonate and extracted with ethyl acetate. The organics were dried and concentrated to give N-(2-amino-phenyl)-2-[1-(4-chloro-phenyl)-2-(2-methoxy-phenyl)-1H-imidazol-4-yl]-acetamide (0.86 g, 1.99 mmol) as an oil.

### Part J-2. Preparation of 2-[1-(4-Chloro-phenyl)-2-(2-methoxy-phenyl)-1H-imidazol-4-ylmethyl]-1H-benzoimidazole

A solution of N-(2-amino-phenyl)-2-[1-(4-chloro-phenyl)-2-(2-methoxy-phenyl)-1H-imidazol-4-yl]-acetamide (0.86 g, 1.99 mmol) in glacial acetic acid (8 mL) was heated at 70° C. for 30 minutes. The mixture was cooled and added dropwise to a saturated aqueous sodium bicarbonate and the pH adjusted to 7 with sodium hydroxide pellets. The mixture was extracted with ethyl acetate, the organics dried and concentrated in vacuum to give an oil. Treatment of the oil with HCl in ether gave 2-[1-(4-chloro-phenyl)-2-(2-methoxy-phenyl)-1H-imidazol-4-ylmethyl]-1H-benzoimidazole (0.44 g, 0.98 mmol) as a white solid.

### Compound J-3

### 2-[2-(2-Methoxy-phenyl)-1-p-tolyl-1H-imidazole-4-ylmethoxymethyl]-1-methyl-1H-benzoimidazole

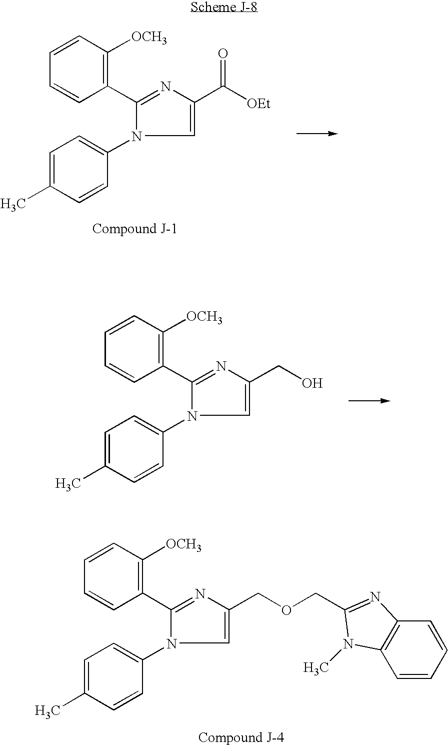

### Part J-1. Preparation of [2-(2-Methoxy-phenyl)-1-p-tolyl-1H-imidazole-4-yl]-methanol

To a −78° C. solution of 2-(2-methoxy-phenyl)-1-p-tolyl-1H-imidazole-4-carboxylic acid ethyl ester (2.0 g, 6.0 mmol) in THF (10 mL) was added dropwise 1M lithium aluminum hydride in ether (6.0 mL, 6.0 mmol). The mixture was warmed to room temperature, stirred for 4 hours and quenched with three drops of methanol. The solvents were removed. The residue was partitioned between methylene chloride and water. The combined organic layers were washed with water, brine, dried over anhydrous Na₂SO₄, filtered and concentrated under vacuum. Column chromatography (SiO₂, ethyl acetate) afforded [2-(2-methoxy-phenyl)-1-p-tolyl-1H-imidazole-4-yl]-methanol (1.1 g, 3.7 mmol) as a solid.

### Part J-2. Preparation of 2-[2-(2-Methoxy-phenyl)-1-p-tolyl-1H-imidazole-4-ylmethoxymethyl]-1-methyl-1H-benzoimidazole

To a solution of [2-(2-methoxy-phenyl)-1-p-tolyl-1H-imidazole-4-yl]-methanol (100 mg, 0.34 mmol) in THF (5 mL) was added NaH (15 mg, 0.34 mmol). The mixture was stirred at room temperature for 30 minutes and 2-chloromethyl-1-methyl-1H-benzoimidazole (61 mg, 0.34 mmol) was added. The mixture was refluxed for 1 hour, cooled to room temperature and quenched with water. The mixture was extracted with ether. The organic layer was washed with water, brine, dried over anhydrous Na₂SO₄, filtered and concentrated under vacuum. Column chromatography (SiO₂, ethyl acetate) afforded 2-[2-(2-methoxy-phenyl)-1-p-tolyl-1H-imidazole-4-ylmethoxymethyl]-1-methyl-1H-benzoimidazole (86 mg, 0.20 mmol) as an oil.

Compounds in the tables herein are prepared in a manner similar as described above and in the general schemes.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

LENGTHY TABLE
The patent application contains a lengthy table section. A copy of the table is available in electronic form from the USPTO web site (http://seqdata.uspto.gov/?pageRequest=docDetail&DocID=US20070281937A1). An electronic copy of the table will also be available from the USPTO upon request and payment of the fee set forth in 37 CFR 1.19(b)(3).

1-38. (canceled)

39. A method of inhibiting Caᵥ1 calcium channel activity in a subject comprising administering to the subject an effective amount of one or more compounds selected from one of the formulae below or a pharmaceutical salt thereof: 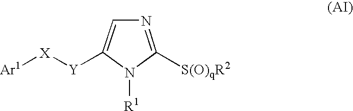
wherein,
Ar¹ is cycloalkyl, aryl, heterocyclyl, or heteroaryl, each optionally substituted with one or more substituents;
X is NR³, C(R³)₂, or O;
Y is C═O or lower alkyl;
R¹ is Ar² or lower alkyl optionally substituted with Ar²;
each Ar² is independently cycloalkyl, aryl, heterocyclyl, or heteroaryl, each optionally substituted with one or more substituents;
q is 0, 1, or 2;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴, and (CH₂)ₙOR⁴;
each R³ is independently H or lower alkyl;
each R⁴ is independently H, lower alkyl, or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl; and
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; 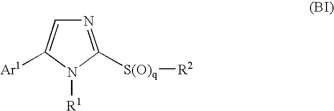
wherein,
Ar¹ is cycloalkyl, aryl, heterocyclyl or heteroaryl, each optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently selected from cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
q is 0, 1, or 2;
each R² is independently selected from (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴ or (CH₂)ₙOR⁴;
each R³ is independently H or lower alkyl;
each R⁴ is independently H, lower alkyl, or (CH₂)ₚAr³;
each Ar³ is cycloalkyl, aryl, heterocyclyl, or heteroaryl, each optionally substituted with one or more substituents, with the proviso that Ar³ is not piperidinyl, tetrahydroquinolinyl or tetrahydroisoquinolinyl;
each Z is independently selected from O or NR³;
each m is 1 or 2;
each n is 2 or 3;
each p is 0 or 1;
each substituent for Ar¹, Ar² and Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl; and
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; 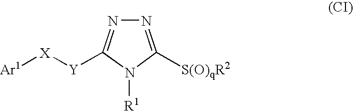
wherein,
Ar¹ is cycloalkyl, aryl, heterocyclyl, or heteroaryl, each optionally substituted with one or more substituents;
X is NR³, C(R³)₂, S, a bond or O, or together with Y forms —CH═CH—;
Y is C═O, a bond, or lower alkyl, or together with X forms —CH═CH—;
R¹ is Ar², alkenyl, or lower alkyl optionally substituted with Ar²;
each Ar² is independently cycloalkyl, aryl, heterocyclyl, or heteroaryl, each optionally substituted with one or more substituents;
q is 0, 1 or 2;
each R² is independently (CH₂)ₘCO₂R³, (CH₂)ₘCOAr³, (CH₂)ₘCONR³R⁴, (CH₂)ₘAr³, (CH₂)₃Ar³, (CH₂)ₙNR³R⁴, (CH₂)ₙOR⁴, (CH₂)ₘCN, alkyl, alkynyl, (CR³R³)ₘCONR³R⁴, Ar⁴, (CR³R³)ₘN(R³)C(O)Ar³, or (CH₂)ₘC(NOH)NH₂;
each R³ is independently H or lower alkyl;
each R⁴ is independently H, lower alkyl, alkoxy, (CH₂), NR⁵R⁶, or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently halogen, CN, NO₂, OR⁶, SR⁶, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, C(O)R⁷, NR⁵C(O)R⁷, S(O)R⁷, or S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl; and
each Ar⁴ is independently C₃-C₆ cycloalkyl, heterocyclyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; 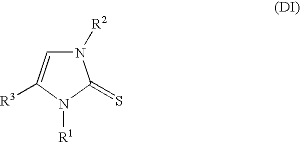
wherein,
R³ is Ar¹ or Ar¹—X—Y wherein,
each Ar¹ is cycloalkyl, aryl, heterocyclyl, or heteroaryl, each optionally substituted with one or more substituents;
X is NR⁴, C(R⁴)₂, or O;
Y is C═O or lower alkyl;
R¹ is Ar² or lower alkyl optionally substituted with Ar²;
each Ar² is independently cycloalkyl, aryl, heterocyclyl, or heteroaryl, each optionally substituted with one or more substituents;
each R² is independently (CH₂)ₘC(O)OR⁴, (CH₂)ₘC(O)Ar³, (CH₂)ₘC(O)NR⁴R⁵, (CH₂)ₙNR⁴R⁵, (CH₂)₃Ar³, or (CH₂)ₘAr³;
each R⁴ is independently H or lower alkyl;
each R⁵ is independently H, lower alkyl, or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁶, SR⁶, S(O)₂OR⁶,NR⁶R⁷, cycloalkyl, C₁-C₂ perfluoroalkyl, C₁-C₂ perfluoroalkoxy, 1,2-methylenedioxy, C(O)OR⁶, C(O)NR⁶R⁷, OC(O)NR⁶R⁷, NR⁶C(O)NR⁶R⁷, C(NR⁶)NR⁶R⁷, NR⁶C(NR⁷)NR⁶R⁷, S(O)₂NR⁶R⁷, R⁸, C(O)R⁸, NR⁶C(O)R⁸, S(O)R⁸, and S(O)₂R⁸;
each R⁶ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently hydrogen, (CH₂)_{q}Ar⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁸ is independently (CH₂)_{q}Ar⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each Ar⁴ is independently C₃-C₆ cycloalkyl, aryl, or heteroaryl, each optionally substituted with one to three substituents independently selected from halogen, OH, C₁-C₄ alkoxy, NH₂, C₁-C₄ alkylamino, C₁-C₄ dialkylamino and C₃-C₆ cycloalkyl; and
q is 0 or 1; 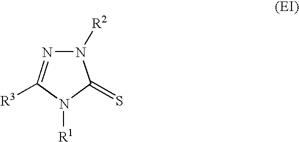
wherein,
R³ is alkyl, alkoxyalkyl, Ar¹ or Ar¹—X—Y wherein,
each Ar¹ is independently cycloalkyl, aryl, heterocyclyl, or heteroaryl, each optionally substituted with one or more substituents;
X is NR⁴, C(R⁴)₂, or O;
Y is C═O or lower alkyl;
R¹ is H, alkenyl, Ar² or lower alkyl optionally substituted with Ar²;
each Ar² is independently cycloalkyl, aryl, heterocyclyl, or heteroaryl, each optionally substituted with one or more substituents;
each R² is independently H, (CH₂)ₘC(O)OR⁴, (CH₂)ₘC(O)Ar³ (CH₂)ₘC(O)NR⁴R⁵, (CH₂)ₘC(O)N(OR⁴)R⁵, (CH₂)ₘCH₂OR⁴, Ar³, (CH₂)ₙNR⁴R⁵, or (CH₂)ₘAr³;
each R⁴ is independently H or lower alkyl;
each R⁵ is independently H, lower alkyl, or (CH₂)ₚAr³;
m is 1 or 2;
n is 2 or 3;
p is 0 or 1;
each Ar³ is cycloalkyl, aryl, heterocyclyl, or heteroaryl, each optionally substituted with one or more substituents;
each substituent for Ar¹, Ar² and Ar³ is independently selected from halogen, CN, NO₂, OR⁶, SR⁶, S(O)₂OR⁶, NR⁶R⁷, cycloalkyl, C₁-C₂ perfluoroalkyl, C₁-C₂ perfluoroalkoxy, 1,2-methylenedioxy, C(O)OR⁶, C(O)NR⁶R⁷, OC(O)NR⁶R⁷, NR⁶C(O)NR⁶R⁷, C(R⁶)NR⁶R⁷, NR⁶C(NR⁷)NR⁶R⁷, S(O)₂NR⁶R⁷, R⁸, C(O)R⁸, NR⁶C(O)R⁸, S(O)R⁸, and S(O)₂R⁸;
each R⁶ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently hydrogen, (CH₂)_{q}Ar⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁸ is independently (CH₂)_{q}Ar⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each Ar⁴ is independently selected from C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and C₃-C₆ cycloalkyl; and
q is 0 or 1; 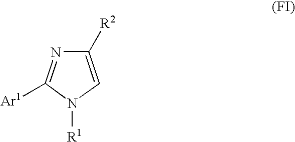
wherein,
Ar¹ is cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently CO₂R³, COAr³, CONR³R⁴, Ar³, or CH₂NR³R⁴;
each R³ is independently H or lower alkyl;
each R⁴ is independently H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each p is independently 0 or 1;
each substituent for Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, CR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)_{q}Ar⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently selected from (CH₂)_{q}Ar⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; and
each q is independently 0 or 1; 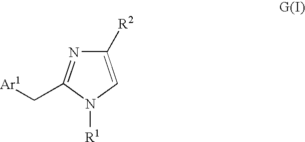
wherein,
Ar¹ is cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
R¹ is Ar² or lower alkyl optionally substituted with Ar²;
Ar² is independently cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently CO₂R³, COAr³, CONR³R⁴, (CH₂)ₘAr³, (CH₂)ₙNR³R⁴ or CH₂OR⁴;
each R³ is independently H or lower alkyl;
each R⁴ is independently H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each m is independently 0 or 1;
each n is independently 1 or 2;
each p is independently 0 or 1;
each substituent for Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)_{q}Ar⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)_{q}Ar⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; and
each q is independently 0 or 1; 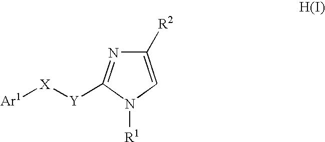
wherein,
Ar¹ is cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
X is NR³, C(R³)₂, or O;
Y is C═O or lower alkyl;
R¹ is Ar² or lower alkyl optionally substituted with Ar²;
Ar² is independently cycloalkyl, aryl, heterocyclyl or heteroaryl, each of which is optionally substituted with one or more substituents selected from halogen, amino, hydroxy, cyano, nitro, carboxylate, alkyl, alkenyl, alkynyl, cycloalkyl, cyclohexyl, alkoxy, mono and di-alkyl amino, phenyl, carboxamide, haloalkyl, haloalkoxy, and alkanoyl;
each R² is independently CO₂R³, COAr³, CONR³R⁴, (CH₂)ₘAr³, CH₂NR³R⁴ or CH₂OR⁴;
each R³ is independently H or lower alkyl;
each R⁴ is independently H, lower alkyl, C(O)OR⁵, C(O)NR⁵R⁶, S(O)₂NR⁵R⁶, C(O)R⁷, S(O)₂R⁷ or (CH₂)ₚAr³;
each Ar³ is independently cycloalkyl, aryl, heterocyclyl, or heteroaryl, each optionally substituted with one or more substituents;
each m is independently 0 or 1;
each p is independently 0 or 1;
each substituent for Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶, S(O)₂NR⁵R⁶, R⁷, C(O)R⁷, NR⁶C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently hydrogen, (CH₂)_{q}Ar⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)_{q}Ar⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy; and
each q is independently 0 or 1; and 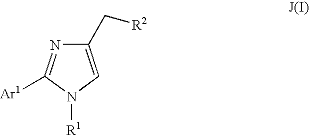
wherein,
Ar¹ is cycloalkyl, aryl, heterocyclyl, or heteroaryl, each optionally substituted with one or more substituents;
R¹ is Ar² or lower alkyl optionally substituted with Ar²;
each Ar² is independently cycloalkyl, aryl, heterocyclyl, or heteroaryl, each optionally substituted with one or more substituents;
each R² is independently (CH₂)ₘCO₂R³, (CH₂)ₘCOAr³, (CH₂)ₘCONR³R⁴, (CH₂)ₘAr³, or (CH₂)ₙNR³R⁴;
each R³ is independently H or lower alkyl;
each R⁴ is independently H, lower alkyl, C(O)OR⁵, C(O)NR⁵R⁶, S(O)₂NR⁵R⁶, C(O)R⁷, S(O)₂)R⁷, or (CH₂)ₚAr³; or
each R³ and R⁴ are taken together with the nitrogen atom to which they are both attached to form a 4-7 membered heterocyclic ring wherein,
one carbon atoms in each heterocyclic ring is optionally a NR⁴, O or S, and each heterocyclic ring is optionally substituted with one or more lower alkyl groups;
each Ar³ is independently cycloalkyl, aryl, heterocyclyl, or heteroaryl, each optionally substituted with one or more substituents;
each m is independently 0 or 1;
each n is independently 1 or 2;
each p is independently 0 or 1;
each substituent for Ar³ is independently selected from halogen, CN, NO₂, OR⁵, SR⁵, S(O)₂OR⁵, NR⁵R⁶, cycloalkyl, C₁-C₂ perfluoroalkyl, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁵)NR⁵R⁶, NR⁵C(NR⁶)NR⁵R⁶,S(O)₂NR⁵R⁶, R⁷, C(O)R⁷, NR⁶C(O)R⁷, S(O)R⁷, and S(O)₂R⁷;
each R⁵ is independently hydrogen or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁶ is independently selected from hydrogen, (CH₂)ₚAr⁴, or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl;
each R⁷ is independently (CH₂)ₚAr⁴ or lower alkyl optionally substituted with one or more substituents independently selected from halogen, OH, C₁-C₄ alkoxy, NH₂, C₁-C₄ alkylamino, C₁-C₄ dialkylamino and C₃-C₆ cycloalkyl; and
each Ar⁴ is independently C₃-C₆ cycloalkyl, aryl or heteroaryl, each optionally substituted with one to three substituents independently selected from halogen, OH, C₁-C₄ alkoxy, NH₂, C₁-C₄ alkylamino, C₁-C₄ dialkylamino and 1,2-methylenedioxy.

40. The method of claim 39, wherein the compound is selected from the compounds in Tables A-J.

41. The method of claim 39, wherein the Caᵥ1 calcium channel is Caᵥ1.2 or Caᵥ1.3.

42. The method of claim 39, wherein the method treats a Caᵥ1 calcium channel-mediated disease or disease symptom.

43. The method of claim 41, wherein the method treats a Caᵥ 1.2 or Caᵥ1.3 calcium channel-mediated disease or disease symptom.

44. The method of claim 42, wherein the Caᵥ1 calcium channel-mediated disease or disease symptom is a cognitive function or nervous system disease or disease symptom.

45. The method of claim 43, wherein the Caᵥ1.2 or Caᵥ1.3 calcium channel-mediated disease or disease symptom is a cognitive function or nervous system disease or disease symptom.

46. The method of claim 42, wherein the Caᵥ1 calcium channel-mediated disease or disease symptom is a cardiovascular disease or disease symptom.

47. The method of claim 43, wherein the Caᵥ1.2 or Caᵥ1.3 calcium channel-mediated disease or disease symptom is a cardiovascular disease or disease symptom.

48. The method of claim 42, wherein the Caᵥ1 calcium channel-mediated disease or disease symptom is selected from angina, congestive heart failure, and myocardial ischemia.

49. The method of claim 43, wherein the Caᵥ1.2 or Caᵥ1.3 calcium channel-mediated disease or disease symptom is selected from angina, congestive heart failure, and myocardial ischemia.

50. The method of claim 42, wherein the Caᵥ1 calcium channel-mediated disease or disease symptom is urinary incontinence or overactive bladder.

51. The method of claim 43, wherein the Caᵥ1.2 or Caᵥ1.3 calcium channel-mediated disease or disease symptom is urinary incontinence or overactive bladder.

52. The method of claim 42, wherein the Caᵥ1 calcium channel-mediated disease or disease symptom is atrial fibrillation.

53. The method of claim 43, wherein the Caᵥ1.2 or Caᵥ1.3 calcium channel-mediated disease or disease symptom is atrial fibrillation.

54. The method of claim 42, wherein the Caᵥ1 calcium channel-mediated disease or disease symptom is hypertension.

55. The method of claim 43, wherein the Caᵥ1.2 or Caᵥ1.3 calcium channel-mediated disease or disease symptom is hypertension.

56. The method of claim 42, wherein the Caᵥ1 calcium channel-mediated disease or disease symptom is selected from angina, hypertension, congestive heart failure, myocardial ischemia, atrial fibrillation, diabetes mellitus, urinary incontinence, overactive bladder, pulmonary disease, cognitive function, and a nervous system disorder.

57. The method of claim 43, wherein the Caᵥ1.2 or Caᵥ1.3 calcium channel-mediated disease or disease symptom is selected from angina, hypertension, congestive heart failure, myocardial ischemia, atrial fibrillation, diabetes mellitus, urinary incontinence, overactive bladder, pulmonary disease, cognitive function, and a nervous system disorder.
